(19) United States
(12) Patent Application Publication
(10) Pub. No.: US 2012/0208809 A1
(43) Pub. Date: Aug. 16, 2012

(54) 9H-PYRROLO[2,3-B: 5,4-C'] DIPYRIDINE AZACARBOLINE DERIVATIVES, PREPARATION THEREOF, AND THERAPEUTIC USE THEREOF

(75) Inventors: Didier Babin, Paris (FR); Olivier Bedel, Paris (FR); Thierry Gouyon, Paris (FR); Serge Mignani, Paris (FR); David Papin, Paris (FR)

(73) Assignee: SANOFI, Paris (FR)

(21) Appl. No.: 13/258,924

(22) PCT Filed: Nov. 30, 2009
(86) PCT No.: PCT/FR09/52330

§ 371 Date: Apr. 27, 2012

(30) Foreign Application Priority Data
Mar. 24, 2009 (FR) 0901368
Oct. 6, 2009 (FR) 0956944

(51) Int. Cl. A61K 31/444; A61K 31/496; A61K 31/5377; C07D 519/00; A61K 31/55; C07F 5/02; C07D 401/04; C07D 213/64; C07D 213/61; A61P 35/00; A61K 31/4545; A61K 31/4375; C07F 5/04; C07F 7/22; C07D 471/14

(52) U.S. Cl. 514/232.8; 546/82; 514/293; 544/361; 514/253.03; 544/126; 514/210.21; 540/597; 514/217.07; 546/15; 514/278; 546/13; 546/258; 546/257; 546/295; 546/303; 546/345; 546/2

(57) ABSTRACT

The invention relates to novel 9H-pyrrolo[2,3-b:5,4-c′]dipyridine azacarbolines of formula (I), where: Z2, Z3, and Z4 are CH, CRa, CRs, or N; R3 is H, Hal; CF3, CHF2; OH, alkoxy; NH2, NH (alkyl), N(alkyl)2; C(O)O alkyl; CONH(alkyl), CON(alkyl)2; C1-C10 alkyl; aryl; heteroaryl; R6 is heteroaryl; Ra is CONH2, CONH alkyl, CONH cycloalkyl; CONH heterocycloalkyl; CON(alkyl)2; CON(alkyl)(heterocycloalkyl); CONHN(alkyl)2; C(O)heterocycloalkyl; Rs is H; Hal, OH; O-alkyl(C1-C10); NH2; N(alkyl(C1-C10) or cycloalkyl(C3-C7))2; NHC(O)R3a; N(alkyl(C1-C10)C(O)R3a; NHS(O2)R3a; N(alkyl)(C1-C10)S(O2)R3a; CO2R3a; SR3a; S(O)R3a; S(O2)R3a; Ra and Rs optionally form a cycle; R3a is selected from among Hal, CF3, C1-C10 alkyl; C3-C7 cycloalkyl; C2-C6 alkenyl; C2-C6 alkynyl; OH; O-alkyl(C1-C10); (C3-C7); heterocycloalkyl (C3-C7); NH2; NH-(alkyl(C1-C10) or cycloalkyl(C3-C7)); N(alkyl(C1-C10) or cycloalkyl(C3-C7))2; NH-(alkyl(C1-C10) or heterocycloalkyl(C3-C7)); N(alkyl(C1-C10) or heterocycloalkyl(C3-C7))2, as well as to the isomers and salts of said substances of formula (I) and to the therapeutic use thereof for treating cancer.

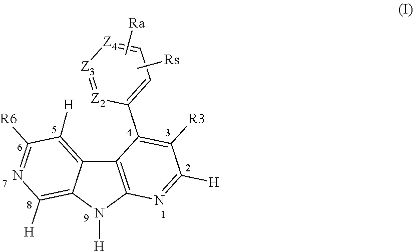

The present invention relates to 9H-pyrrolo[2,3-b:5,4-c′]dipyridine α-aza-β-carboline derivatives, to their preparation and to their therapeutic use. The α-aza-β-carbolines are defined by the 1,7-diazacarbazole or 8-aza-β-carboline derivatives; in the French IUPAC nomenclature (use of ACD/Name 12.00), the name of this tricyclic unit is 9H-pyrrolo[2,3-b:5,4-c′]dipyridine.

The present invention is directed towards compounds that act on protein kinases, for instance: CHK1, CDK1, CDK2, dyrk2, Flt3, GSK3 beta, MNK2, PDGFR beta, PI3K, PIM1, PIM2, PIM3, PLK, TrkB, all of which are involved in the development of cancers. More particularly, the present invention is directed towards compounds that act on a target known as Pim, which is involved in the development of cancers.

The Pim kinases, including Pim-1, Pim-2 and Pim-3, form a distinct family of serine/threonine kinases, and play a functional role in cell growth, differentiation and apoptosis. One of the mechanisms via which the Pim kinases can increase the survival of cancer cells and promote the development of cancer proceeds via modulation of the activity of BAD, a key regulator of apoptosis. The Pim kinases are mutually highly homogeneous and display similar oncogenic behaviour.

Clinical reports underline the importance of the role of the Pim kinases in the development of human cancers:

The Pim kinases, in particular Pim-1 and Pim-2, have been found to be abnormally expressed in a large number of malignant haematological diseases. Amson et al. report the overexpression of Pim-1 in acute myeloid leukaemia and acute lymphoid leukaemia, and that the overexpression of Pim-1 appears to result from an untoward activation in various leukaemias (Proc. Natl. Acad. Sci., Vol. 86, 8857-8861 (1989)). Studies have demonstrated the overexpression of Pim-1 in primitive and metastatic lymphoma of the CNS, an aggressive form of non-Hodgkin lymphoma (Rubenstein et al., Blood, Vol. 107, No. 9, 3716-3723 (2006)). Hüttmann et al. have also discovered an overexpression of Pim-2 in chronic B-cell lymphocytic leukaemia and suggest that upregulation of Pim-2 may be associated with a more aggressive development of the disease (Leukemia, 20, 1774-1782 (2006)). An abnormal expression of Pim-1 and of Pim-2 has been associated with multiple myeloma (Claudio et al., Blood, v. 100. No. 6, 2175-2186 (2002)).

Hypermutations of Pim-1 have been identified in diffuse large-cell lymphomas (Pasqualucci et al., Nature, vol. 412, 2001, p. 341-346 (2001)) and in standard and nodular Hodgkin lymphoma with a lymphocytic predominance (Lisa et al., Blood, Vol. 108, No. 3, 1013-1020 (2006)).

Many studies have also linked an abnormal expression of the Pim kinases to various non-haematological human cancers (prostate, pancreas, head and neck, etc.) and their presence is often associated with a more aggressive phenotype. For example, Pim-1 and Pim-2 have both been implicated in prostate cancer (Chen et al., Mol. Cancer Res., 3(8) 443-451 (2005)). Valdman et al. have demonstrated upregulation of Pim-1 in the case of patients suffering from a prostate carcinoma and in high-grade intraepithelial prostate neoplasia (precancerous lesions) (The Prostate, (60) 367-371 (2004)), whereas Dai et al. suggest that the overexpression of Pim-2 in prostate cancer is associated with more aggressive clinical characteristics (The Prostate, 65:276-286 (2005)). Xie et al. have discovered that the 44 kDa Pim-1 (Pim-1L) was significantly upregulated in samples of human prostate tumour, and indicate that Pim-1L has an anti-apoptotic effect on human prostate cancer cells in response to chemotherapeutic drugs (Oncogene, 25, 70-78 (2006)).

Pim-2 is linked to perineural invasion (PNI), during which the cancer cells become wound around nerves, which is often found in certain cancers such as prostate cancer, pancreatic cancer, cancer of the bile ducts and cancer of the head and neck (Ayala et al., Cancer Research, 64, 6082-6090 (2004)). According to Li at al., Pim-3 is aberrantly expressed in human and murine hepatocarcinomas and human pancreatic cancer tissues (Cancer Res. 66 (13), 6741-6747 (2006)). An aberrant expression of Pim-3 has also been observed in gastric adenoma and the metastatic sites of gastric carcinoma (Zheng et al., J. Cancer Res. Clin. Oncol., 134:481-488 (2008)).

Together, these reports suggest that Pim kinase inhibitors are useful for treating cancer, especially leukaemias, lymphomas, myelomas and various solid tumours, especially cancers of the head and neck, bowel cancer, prostate cancer, pancreatic cancer, liver cancer and oral cancer, for example. Insofar as cancer remains a disease for which the existing treatments are insufficient, it is manifestly necessary to identify novel Pim kinase inhibitors that are effective for treating cancer.

Among the patent applications claiming compounds of the azacarboline class, which is the subject of our invention, mention may be made of the following documents:

Patent application WO 2007/044 779 describes 9H-pyrrolo[2,3-b:5,4-c′]dipyridines or α-aza-β-carbolines of the following general formula, which is partially restricted, relative to the application as published:

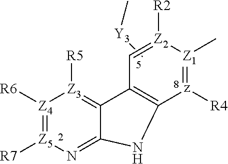

in which
Z5, Z4 and Z3 may represent C and
Z and Z2 may also represent C,
Z1 may finally represent C or N and
R2 may represent a carbon bond or an alkylene radical, each possibly being substituted with several possibilities, including heteroaryloxy, heteroaryl(C1-C5)alkyl, heteroaryls and heterobicycloaryls.

The preparation process and all the examples of this application are limited to derivatives substituted in positions 2 and 8 and optionally in position 5.

Patent EP 1 209 158 claims compounds having the following formula:

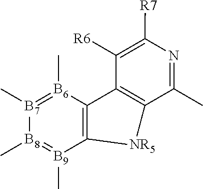

in which B6, B7, B8, B9 may represent C or N, but R7 never represents a heteroaryl. The activity of the compounds of this invention is particularly directed towards the treatment of heart problems.

The present invention relates to compounds of general formula (I) below:

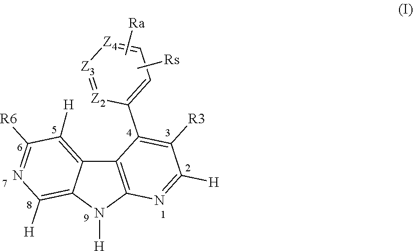

in which
Z₂, Z₃, Z₄, which may be identical or different, represent CH, CRa, CRs or N;
R3 is chosen from:
   1. H;
   2. halogen (F, Cl, Br, I);
   3. —CF₃, —CHF₂,
   4. —OH
   5. alkoxy in which the alkyl part is optionally mono-, di- or trisubstituted;
   6. —NH2, —NH(alkyl), —N(alkyl)₂ in which the alkyl part is optionally mono-, di- or trisubstituted;
   7. —C(O)Oalkyl optionally mono-, di- or trisubstituted;
   8. —CONH(alkyl), CON(alkyl)₂ in which the alkyl part is optionally mono-, di- or trisubstituted;
   9. linear, branched or cyclic C₁-C₁₀ alkyl optionally comprising a heteroatom and optionally mono-, di- or trisubstituted;
   10. aryl or heteroaryl optionally mono-, di- or trisubstituted;
R6 being a heteroaryl (5- or 6-membered with 1 to 4 heteroatoms chosen from N, S and O) linked to the azacarboline unit either via a C or via an N belonging to R6, R6 being optionally mono- or polysubstituted;
Ra being necessarily chosen from:
   1. —CONH₂,
   2. —CONHalkyl, CONNcycloalkyl optionally mono-, di- or trisubstituted;
   3. —CONHheterocycloalkyl optionally mono-, di- or trisubstituted;
   4. —CON(alkyl)₂ optionally mono-, di- or trisubstituted;
   5. —CON(alkyl)(heterocycloalkyl) optionally mono-, di- or trisubstituted;
   6. —CONHN(alkyl)₂ in which the alkyl part is optionally mono-, di- or trisubstituted;
   7. —C(O)heterocycloalkyl, the heterocycloalkyl radical containing at least one nitrogen atom linked to C(O); and being optionally mono-, di- or trisubstituted;
Rs being chosen from the following groups:
   1. H;
   2. F; Cl; Br; I
   3. —OH;
   4. linear or branched —O—(C₁-C₁₀)alkyl optionally mono- or polysubstituted;
   5. —NH₂;
   6. —N(alkyl(C₁-C₁₀) or cycloalkyl(C₃-C₇))₂, each group being optionally mono- or polysubstituted;
   7. —NHC(O)R3a;
   8. —N(alkyl(C₁-C₁₀)C(O)R3a;
   9. —NHS(O₂)R3a;
   10. —N(alkyl(C₁-C₁₀)S(O₂)R3a;
   11. —CO₂R3a;
   12. —SR3a; —S(O)R3a; —S(O2)R3a
      Ra and Rs possibly forming a 4- to 7-membered ring substituted with an oxo radical, comprising at least one nitrogen atom and optionally another heteroatom chosen from N, O and S and optionally substituted with one or more radicals chosen from oxo, F, Cl, Br, CF3, CHF2, alkyl, OH, Oalkyl, NO2, NH2, NHAlk and N(Alk)2 radicals;
      R3a being chosen from:
1. F; Cl; Br; I
2. —CF3;
3. linear or branched C1-C10 alkyl
4. —C3-C7 cycloalkyl;
5. —C2-C6 alkenyl;
6. —C2-C6 alkynyl;
7. —OH;
8. linear or branched (C1-C10) or cyclic (C3-C7)-O-alkyl;
9. heterocycloalkyl (C3-C7);
10. —NH2;
11. —NH-(alkyl(C1-C10) or cycloalkyl(C3-C7));
12. —N(alkyl(C1-C10) or cycloalkyl(C3-C7))2;
13. —NH-(alkyl(C1-C10) or heterocycloalkyl (C3-C7));
14. —N(alkyl(C1-C10) or heterocycloalkyl (C3-C7))2;
   the said products of formula (I) being in any possible racemic, enantiomeric or diastereoisomeric isomer form, and also the addition salts with mineral and organic acids or with mineral and organic bases of the said products of formula (I).

The products of formula (I) according to the present invention are such that the ring hereinbelow:

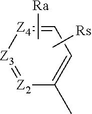

bears only one substituent Ra and only one substituent Rs as defined above. Ra and Rs being linked to the carbon chain units or to Z2, Z3 or Z4 when they represent a carbon chain unit.

One subject of the present invention is thus the compounds as defined above, characterized in that the possible substituents of R3, R6 and Ra are chosen from the groups R2a, R2b and R2c chosen, independently of each other, from:
1. F;
2. Cl;
3. Br;
4. I;
5. —CF₃; —CHF2
6. linear or branched C1-C10 alkyl optionally mono- or polysubstituted;
7. —C₃-C₇ cycloalkyl optionally mono- or polysubstituted;
8. —OH;
9. linear or branched —O—(C₁-C₁₀)alkyl optionally mono- or polysubstituted;
10. —O-cycloalkyl (C₃-C₇) optionally mono- or polysubstituted;
11. —O-aryl optionally mono- or polysubstituted;
12. aryl optionally mono- or polysubstituted;
13. heteroaryl optionally mono- or polysubstituted;
14. heterocycloalkyl optionally mono- or polysubstituted;
15. —NO₂,
16. —NH₂;
17. —NH-(alkyl(C₁-C₁₀) or cycloalkyl(C₃-C₇) or heterocycloalkyl), each group optionally mono- or polysubstituted;
18. —N(alkyl(C₁-C₁₀) or cycloalkyl(C₃-C₇))₂, each group being optionally mono- or polysubstituted;
19. —NHaryl or NH heteroaryl optionally mono- or polysubstituted
20. —NHC(O)substituted;
21. —N(alkyl(C₁-C₁₀)C(O)substituted;
22. —NHS(O₂)substituted;
23. —N(alkyl(C₁-C₁₀)S(O₂)substituted;
24. —CO₂ substituted;
25. —S substituted;
26. —S(O₂)substituted;
27. —S(O) substituted;
28. oxo (double bond O);
   the said products of formula (I) being in any possible racemic, enantiomeric or diastereoisomeric isomer form, and also the addition salts with mineral and organic acids or with mineral and organic bases of the said products of formula (I).

One subject of the present invention is thus the compounds as defined above, characterized in that the optional substituents of all the substituted groups and of the groups Rs, R2a, R2b and R2c or the groups are chosen from:
1. F; Cl; Br; I
2. —CF₃;
3. linear or branched C1-C10 alkyl
4. —C₃-C₇ cycloalkyl;
5. —C₂-C₆ alkenyl;
6. —C₂-C₆ alkynyl;
7. —OH;
8. linear or branched (C1-C10) or cyclic (C3-C7)-O-alkyl;
9. heterocycloalkyl (C3-C7);
10. —NH₂;
11. —NH-(alkyl(C₁-C₁₀) or cycloalkyl(C₃-C₇));
12. —N(alkyl(C₁-C₁₀) or cycloalkyl(C₃-C₇))₂;
13. —NH-(alkyl(C₁-C₁₀) or heterocycloalkyl (C₃-C₇));
14. —N(alkyl(C₁-C₁₀) or heterocycloalkyl (C₃-C₇))₂;
   the said products of formula (I) being in any possible racemic, enantiomeric or diastereoisomeric isomer form, and also the addition salts with mineral and organic acids or with mineral and organic bases of the said products of formula (I).

One subject of the present invention is thus the compounds of general formula (I) as defined above, in which
Z₂, Z₃, Z₄, which may be identical or different, represent CH, CRa, CRs or N;
R3 is chosen from:
   1. H;
   2. halogen (F, Cl, Br, I);
   3. —CF₃, —CHF₂;
   4. —OH
   5. alkoxy in which the alkyl part is optionally mono-, di- or trisubstituted with R2a, R2b, R2c;
   6. —NH₂, —NH(alkyl), —N(alkyl), in which the alkyl part is optionally mono-, di- or trisubstituted with R2a, R2b, R2c;
   7. —C(O)Oalkyl optionally mono-, di- or trisubstituted with R2a, R2b, R2c;
   8. —CONH(alkyl), CON(alkyl)₂ in which the alkyl part is optionally mono-, di- or trisubstituted with R2a, R2b, R2c;
   9. linear, branched or cyclic C₁-C₁₀ alkyl optionally comprising a heteroatom and optionally mono-, di- or trisubstituted with R2a, R2b, R2c;
   10. aryl or heteroaryl optionally mono-, di- or trisubstituted with R2a, R2b, R2c;
      R6 being a heteroaryl (5- or 6-membered with 1 to 4 heteroatoms chosen from N, S and O) linked to the azacarboline unit either via a C or via an N belonging to R6, R6 being optionally mono- or polysubstituted with R2a, R2b, R2c;
Ra necessarily being:
   1. —CONH₂,
   2. —CONHalkyl, CONHcycloalkyl optionally mono-, di- or trisubstituted with R2a, R2b, R2c;
   3. —CONHheterocycloalkyl optionally mono-, di- or trisubstituted with R2a, R2b, R2c;
   4. —CON(alkyl)₂ optionally mono-, di- or trisubstituted with R2a, R2b, R2c;
   5. —CON(alkyl)(heterocycloalkyl) optionally mono-, di- or trisubstituted with R2a, R2b, R2c;
   6. —CONHN(alkyl), in which the alkyl part is optionally mono-, di- or trisubstituted with R2a, R2b, R2c;
   7. —C(O)heterocycloalkyl, the heterocycloalkyl radical containing at least one nitrogen atom linked to C(O); and being optionally mono-, di- or trisubstituted;
      Rs being chosen from the following groups:
   1. H;
   2. F; Cl; Br; I
   3. —OH;
   4. linear or branched —O—(C₁-C₁₀)alkyl optionally mono- or polysubstituted with identical or different groups R3a;
   5. —NH₂;
   6. —N(alkyl(C₁-C₁₀) or cycloalkyl(C₃-C₇))₂, each group being optionally mono- or polysubstituted with identical or different groups R3a;
   7. —NHC(O)R3a;
   8. —N(alkyl(C₁-C₁₀)C(O)R3a;
   9. —NHS(O₂)R3a;
   10. —N(alkyl(C₁-C₁₀)S(O₂)R3a;
   11. —CO₂R3a;
   12. —SR3a; —S(O)R3a; —S(O₂)R3a
      Ra and Rs may optionally form a 5- to 6-membered ring substituted with an oxo radical and comprising at least one nitrogen atom and optionally substituted with one or more radicals chosen from oxo, F, Cl, Br, I, CF3, CHF2, alkyl, OH, Oalkyl, NO2, NH2, NHAlk and N(Alk)2 radicals;

The groups R2a, R2b or R2c are chosen, independently of each other, from:
1. F;
2. Cl;
3. Br;
4. I;
5. —CF₃; —CHF₂
6. linear or branched C1-C10 alkyl optionally mono- or polysubstituted with identical or different groups R3a;
7. —C₃-C₇ cycloalkyl optionally mono- or polysubstituted with identical or different groups R3a;
8. —OH;
9. linear or branched —O—(C₁-C₁₀)alkyl optionally mono- or polysubstituted with identical or different groups R3a;
10. —O-cycloalkyl (C₃-C₇) optionally mono- or polysubstituted with identical or different groups R3a;
11. —O-aryl optionally mono- or polysubstituted with different groups R3a;
12. aryl optionally mono- or polysubstituted with identical or different groups R3a;
13. heteroaryl optionally mono- or polysubstituted with identical or different groups R3a;
14. heterocycloalkyl optionally mono- or polysubstituted with identical or different groups R3a;
15. —NO₂
16. —NH₂;
17. —NH-(alkyl(C₁-C₁₀) or cycloalkyl(C₃-C₇) or heterocycloalkyl), each group optionally mono- or polysubstituted with identical or different groups R3a;
18. —N(alkyl(C₁-C₁₀) or cycloalkyl(C₃-C₇))₂, each group being optionally mono- or polysubstituted with identical or different groups R3a;
19. NHaryl or NH heteroaryl optionally mono- or polysubstituted with identical or different groups R3a
20. NHC(O)R3a;
21. N(alkyl(C₁-C₁₀)C(O)R3a;
22. NHS(O₂)R3a;
23. N(alkyl(C₁-C₁₀)S(O₂)R3a;
24. CO₂R3a;
25. SR3a; S(O)R3a; S(O₂)R3a;
26. N(alkyl(C₁-C₁₀) or cycloalkyl (C₃-C₇))₂, each group being optionally mono- or polysubstituted with identical or different groups R3a;
27. oxo (double bond O);

The possible substituents of the groups R2a, R2b and R2c or the groups R3a are chosen from:
2. F; Cl; Br; I
   3. —CF₃;
4. linear or branched C1-C10 alkyl
5. —C₃-C₇ cycloalkyl;
6. —C₂-C₆ alkenyl;
7. —C₂-C₆ alkynyl;
8. —OH;
9. linear or branched (C1-C10) or cyclic (C3-C7)-O-alkyl;
10. heterocycloalkyl (C₃-C₇);
11. —NH₂;
12. —NH-(alkyl(C₁-C₁₀) or cycloalkyl(C₃-C₇));
13. —N(alkyl(C₁-C₁₀) or cycloalkyl(C₃-C₇))₂;
14. —NH-(alkyl(C₁-C₁₀) or heterocycloalkyl (C₃-C₇));
15. —N(alkyl(C₁-C₁₀) or heterocycloalkyl (C₃-C₇))₂.

In the context of the present invention, positions 2 and 8 should not be substituted, in contrast with the prior art documents.

The substituents Ra and Rs as defined above are, respectively, and without preference, linked to any of the carbons of the ring below:

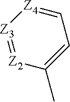

in which Z2, Z3 and Z4 have the meanings indicated above.

A subject of the present invention is particularly the products of formula I belonging to formula Ia:

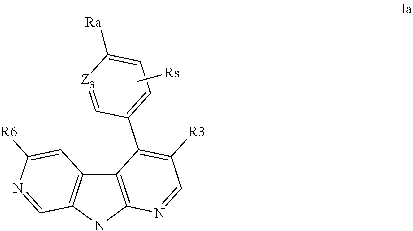

in which Z2 represents CH, Z4 represents —C—Ra and R3, R6, Ra and Rs have any of the meanings given above and Z3 represents CH or N,
the said products of formula (I) being in any possible racemic, enantiomeric or diastereoisomeric isomer form, and also the addition salts with mineral and organic acids or with mineral and organic bases of the said products of formula (I).

A subject of the present invention is particularly the products of formula I belonging to formula Ib:

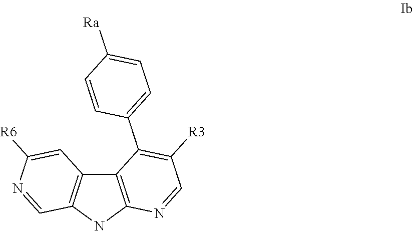

in which Rs represents a hydrogen atom. Z2 and Z3 represent CH, Z4 represents —C—Ra and R3, R6 and Ra have any of the meanings given above, the said products of formula (I) being in any possible racemic, enantiomeric or diastereoisomeric isomer form, and also the addition salts with mineral and organic acids or with mineral and organic bases of the said products of formula (I).

A subject of the present invention is particularly the products of formula I belonging to formula Ic:

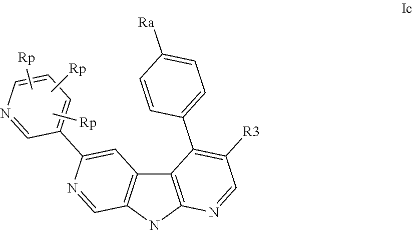

in which Rs represents a hydrogen atom, Z2 and Z3 represent CH, Z4 represents —C—Ra, R6 represents a pyridyl radical optionally mono- or polysubstituted with one or more identical or different radicals Rp chosen from F, Cl, Br, I, CF3, CHF2, alkyl, OH, Oalkyl, NO2, NH2, NHAlk and N(Alk)2 radicals and R3 and Ra have any of the meanings given above, the said products of formula (I) being in any possible racemic, enantiomeric or diastereoisomeric isomer form, and also the addition salts with mineral and organic acids or with mineral and organic bases of the said products of formula (I).

A subject of the present invention is particularly the products of formula I belonging to formula Id:

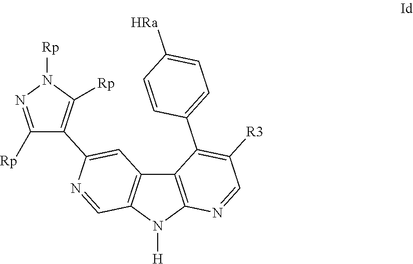

in which Rs represents a hydrogen atom, Z2 and Z3 represent CH, Z4 represents —C-Ra, R6 represents a pyrazolyl radical optionally mono- or polysubstituted with one or more identical or different radicals Rp chosen from F, Cl, Br, I, CF3, CHF2, alkyl, OH, Oalkyl, NO2, NH2, NHAlk and N(Alk)2 radicals and R3 and Ra have any of the meanings given above, the said products of formula (I) being in any possible racemic, enantiomeric or diastereoisomeric isomer form, and also the addition salts with mineral and organic acids or with mineral and organic bases of the said products of formula (I).

The following terms used herein above or hereinbelow have the following meanings:
Alkyl, alkyl(C₁-C₁₀) or C₁-C₁₀ alkyl means any linear or branched, saturated carbon chain of 1 to 10 carbons.
Aryl means phenyl or naphthyl.
Heteroaryl means any 5- or 6-membered aromatic monocycle containing at least one heteroatom (N, O, S), especially: pyridine, pyrimidine, imidazole, pyrazole, triazole, thiophene, furan, thiazole, oxazole, etc., and also aromatic bicyclic systems containing at least one heteroatom (N, O, S), especially indole, benzimidazole, azaindole, benzofuran, benzothiophene, quinoline, tetrazole
Heterocycloalkyl means any non-aromatic monocycle or bicycle (spiro or non-spiro) containing at least one heteroatom (N, O, S in the different possible oxidation states) with or without unsaturation, especially: morpholine, piperazine, piperidine, pyrrolidine, oxetane, epoxide, dioxane, imidazolone, imidazolinedione, 7-oxa-bicyclo-[2.2.1]heptane, azetidine, azepine, hexahydropyrrolo[3,4-b]pyrrole, hexahydropyrrolo[2,3-b]pyrrole, hexahydropyrrolo[3,4-c]pyrrole, hexahydropyrrolo-[2,3-c]pyrrole, 2,7-diazaspiro[4,4]nonane, 2,6-diazaspiro[4,4]nonane, 3,6-diazaspiro[4,4]nonane, 3,7-diazaspiro[4,4]nonane, 3,8-diazaspiro[4,4]nonane, 3,9-diazaspiro[4,4]nonane, 4,6-diazaspiro[4,4]nonane, 4,7-diazaspiro[4,4]nonane, 4,8-diazaspiro[4,4]nonane, 4,9-diazaspiro[4,4]nonane, 1,6-diazaspiro[4,4]nonane, 1,7-diazaspiro[4,4]nonane, 1,8-diazaspiro[4,4]nonane, 1,9-diazaspiro[4,4]nonane, octahydropyrrolo[3,4-c]pyridine, octahydropyrrolo[3,4-d]pyridine, octahydropyrrolo-[3,4-e]pyridine, octahydropyrrolo[2,3-c]pyridine, octahydropyrrolo[2,3-d]pyridine, octahydropyrrolo[2,3-e]pyridine, and especially hexahydropyrrolo[3,4-b]pyrrole, 2,7-diazaspiro[4,4]nonane and octahydropyrrolo[3,4-c]pyridine.
Cycloalkyl(C₃-C₇) means any non-aromatic ring formed solely from carbon atoms, especially cyclopropane, cyclobutane, cyclopentane, cyclohexane, cycloheptane; but also possibly bearing an unsaturation, for example cyclopentene, cyclohexene, cycloheptene or bicyclo[2.2.1]heptane.
C₁-C₁₀ alkylhydroxy means any linear or branched, saturated carbon chain of 1 to 10 carbons bearing at least one hydroxyl group (OH).
C₁-C₁₀ alkoxy means any linear or branched, saturated carbon chain of 1 to 10 carbons bearing at least one ether function (C—O—C).
C₁-C₁₀ alkylamino means any linear or branched, saturated carbon chain of 1 to 10 carbons bearing at least one amine (primary, secondary or tertiary) function

A subject of the present invention is especially the products of formula (I) as defined above, the names of which are as follows:
4-[3-fluoro-6-(pyridin-3-yl)-9H-pyrrolo[2,3-b:5,4-c′]dipyridin-4-yl]-N-(1H-tetrazol-5-yl-methyl)benzamide
[(3R)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
{4-[3-fluoro-6-(pyridin-3-yl)-9H-pyrrolo[2,3-b:5,4-c′]dipyridin-4-yl]phenyl}[(3aS,6aS)-5-methylhexahydropyrrolo[3,4-b]pyrrol-1(2H)-yl]methanone
N-[2-(acetylamino)ethyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[3-(2-oxopyrrolidin-1-yl)propyl]benzamide
4-[3-fluoro-6-(pyridin-3-yl)-9H-pyrrolo[2,3-b:5,4-c′]dipyridin-4-yl]-N-[2-(phenyl-amino)ethyl]benzamide
N-[(1-ethylpyrrolidin-2-yl)methyl]-4-[3-fluoro-6-(pyridin-3-yl)-9H-pyrrolo[2,3-b: 5,4-c′]dipyridin-4-yl]benzamide
N-[3-(dimethylamino)-2,2-dimethylpropyl]-4-[3-fluoro-6-(pyridin-3-yl)-9H-pyrrolo[2,3-b:5,4-c′]dipyridin-4-yl]benzamide
N-{[(2S)-1-ethylpyrrolidin-2-yl]methyl}-4-[3-fluoro-6-(pyridin-3-yl)-9H-pyrrolo[2,3-b:5,4-c′]dipyridin-4-yl]benzamide
N-(1-ethylpiperidin-3-yl)-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(2-methylpiperidin-1-yl)ethyl]benzamide
4-[3-fluoro-6-(pyridin-3-yl)-9H-pyrrolo[2,3-b:5,4-c′]dipyridin-4-yl]-N-(1-methylazetidin-3-yl)benzamide
[3-(dimethylamino)piperidin-1-yl]{-4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-[2-methyl-2-(pyrrolidin-1-yl)propyl]benzamide
N-[3-(dimethylamino)propyl]-4-[3-fluoro-6-(pyridin-3-yl)-9H-pyrrolo[2,3-b:5,4-c′]dipyridin-4-yl]-N-methylbenzamide
N-[2-(azepan-1-yl)ethyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(1-methylpiperidin-4-yl)ethyl]benzamide
4-[3-fluoro-6-(pyridin-3-yl)-9H-pyrrolo[2,3-b:5,4-c′]dipyridin-4-yl]-N-{2-[(methyl-sulfonyl)amino]ethyl}benzamide
4-[3-fluoro-6-(pyridin-3-yl)-9H-pyrrolo[2,3-b:5,4-c′]dipyridin-4-yl]-N-[2-(pyrrolidin-1-yl)propyl]benzamide
4-[3-fluoro-6-(pyridin-3-yl)-9H-pyrrolo[2,3-b:5,4-c′]dipyridin-4-yl]-N-methyl-N-[(1-methylpiperidin-2-yl) methyl]benzamide
4-[3-fluoro-6-(pyridin-3-yl)-9H-pyrrolo[2,3-b:5,4-c′]dipyridin-4-yl]-N-[2-(1-methylpyrrolidin-2-yl)ethyl]benzamide
N-[2-(dipropan-2-ylamino)ethyl]-4-[3-fluoro-6-(pyridin-3-yl)-9H-pyrrolo[2,3-b:5,4-c′]dipyridin-4-yl]benzamide
N-[2-(dimethylamino)ethyl]-N-ethyl-4-[3-fluoro-6-(pyridin-3-yl)-9H-pyrrolo[2,3-b:5,4-c′]dipyridin-4-yl]benzamide
N-[1-(dimethylamino)propan-2-yl]-4-[3-fluoro-6-(pyridin-3-yl)-9H-pyrrolo[2,3-b:5,4-c′]dipyridin-4-yl]benzamide
[(3S)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-methyl-N-(1-methylpyrrolidin-3-yl)benzamide
N-[2-(diethylamino)ethyl]-4-[3-fluoro-6-(pyridin-3-yl)-9H-pyrrolo[2,3-b:5,4-c′]dipyridin-4-yl]-N-methylbenzamide
{4-[3-fluoro-6-(pyridin-3-yl)-9H-pyrrolo[2,3-b:5,4-c′]dipyridin-4-yl]phenyl}[4-(2-methoxyethyl)piperazin-1-yl]methanone
4-[3-fluoro-6-(pyridin-3-yl)-9H-pyrrolo[2,3-b:5,4-c′]dipyridin-4-yl]-N-[(3-methyl-1H-pyrazol-4-yl)methyl]benzamide
{4-[3-fluoro-6-(pyridin-3-yl)-9H-pyrrolo[2,3-b:5,4-c′]dipyridin-4-yl]phenyl}(2-methyl-octahydro-5H-pyrrolo[3,4-c]pyridin-5-yl)methanone
N-[4-(dimethylamino)butyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-(1H-imidazol-2-ylmethyl)benzamide
{4-[3-fluoro-6-(pyridin-3-yl)-9H-pyrrolo[2,3-b:5,4-c′]dipyridin-4-yl]phenyl}(7-methyl-4-2,7-diazaspiro[4.4]non-2-yl)methanone
4-[3-fluoro-6-(pyridin-3-yl)-9H-pyrrolo[2,3-b:5,4-c′]dipyridin-4-yl]-N-[2-(pyridin-2-yl-amino)ethyl]benzamide
N-ethyl-4-[3-fluoro-6-(pyridin-3-yl)-9H-pyrrolo[2,3-b:5,4-c′]dipyridin-4-yl]-N-[(1-methylpyrrolidin-3-yl)methyl]benzamide
1,3′-bipyrrolidin-1′-yl{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-methyl-N-(1-methylpiperidin-4-yl)benzamide
4-[3-fluoro-6-(pyridin-3-yl)-9H-pyrrolo[2,3-b:5,4-c′]dipyridin-4-yl]-N-[(2-hydroxypyridin-4-yl)methyl]benzamide
N-[2-(ethylamino)ethyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(methyl-amino)ethyl]benzamide
N-[(1-aminocyclopropyl)methyl]-4-[3-fluoro-6-(pyridin-3-yl)-9H-pyrrolo[2,3-b:5,4-c′]dipyridin-4-yl]benzamide
N-(3-amino-2,2-difluoropropyl)-4-[3-fluoro-6-(pyridin-3-yl)-9H-pyrrolo[2,3-b:5,4-c′]dipyridin-4-yl]benzamide
N-(2-amino-3,3,3-trifluoro-2-methylpropyl)-4-[3-fluoro-6-(pyridin-3-yl)-9H-pyrrolo-[2,3-b:5,4-c′]dipyridin-4-yl]benzamide
N-[(1R,2R)-2-aminocyclohexyl]-4-[3-fluoro-6-(pyridin-3-yl)-9H-pyrrolo[2,3-b:5,4-c′]dipyridin-4-yl]benzamide
N-[(1S,2S)-2-aminocyclohexyl]-4-[3-fluoro-6-(pyridin-3-yl)-9H-pyrrolo[2,3-b:5,4-c′]dipyridin-4-yl]benzamide
N-[(1S,2S)-2-aminocyclopentyl]-4-[3-fluoro-6-(pyridin-3-yl)-9H-pyrrolo[2,3-b:5,4-c′]dipyridin-4-yl]benzamide
N-[(1R,2R)-2-aminocyclopentyl]-4-[3-fluoro-6-(pyridin-3-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-(4-methylpiperazin-1-yl)benzamide
4-[3-fluoro-6-(1-methyl-1H-pyrazol-4-yl)-9H-pyrrolo[2,3-b:5,4-c′]dipyridin-4-yl]-N-(1-methylpiperidin-4-yl)benzamide
4-[3-fluoro-6-(1-methyl-1H-pyrazol-4-yl)-9H-pyrrolo[2,3-b:5,4-c′]dipyridin-4-yl]-N-{2-[(3R)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3S)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-(2-hydroxyethyl)benzamide
N-[(1S,2S)-2-aminocyclohexyl]-4-[3-fluoro-6-(1-methyl-1H-pyrazol-4-yl)-9H-pyrrolo-[2,3-b:5,4-c′]dipyridin-4-yl]benzamide
N-[(1S,2S)-2-(diethylamino)cyclohexyl]-4-[3-fluoro-6-(1-methyl-1H-pyrazol-4-yl)-9H-pyrrolo[2,3-b:5,4-c′]dipyridin-4-yl]benzamide
N-[(1S,2S)-2-(ethylamino)cyclohexyl]-4-[3-fluoro-6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]benzamide
4-[6-(5-chloro-1-methyl-1H-pyrazol-4-yl)-3-fluoro-9H-pyrrolo[2,3-b:5,4-c′]dipyridin-4-yl]-N-(4-methylpiperazin-1-yl)benzamide
4-[6-(5-chloro-1-methyl-1H-pyrazol-4-yl)-3-fluoro-9H-pyrrolo[2,3-b:5,4-c′]dipyridin-4-yl]-N-(1-methylpiperidin-4-yl)benzamide
N-[2-(dimethylamino)ethyl]-4-[3-(2-methoxyethoxy)-6-(pyridin-3-yl)-9H-pyrrolo[2,3-b:5,4-c′]dipyridin-4-yl]benzamide
N-[2-(dimethylamino)ethyl]-5-[3-fluoro-6-(pyridin-3-yl)-9H-pyrrolo[2,3-b:5,4-c]dipyridin-4-yl]pyridine-2-carboxamide
N-[2-(dimethylamino)ethyl]-2-fluoro-4-[3-fluoro-6-(pyridin-3-yl)-9H-pyrrolo[2,3-b:5,4-c′]dipyridin-4-yl]benzamide.

A subject of the present invention is also the products of formula (I) as defined above, the names of which are as follows:
N-(2-aminoethyl)-4-[3-(2-methoxyethoxy)-6-(1-methyl-1H-pyrazol-4-yl)-9H-pyrrolo-[2,3-b:5,4-c′]dipyridin-4-yl]benzamide
N-(2-aminoethyl)-4-[6-(1-methyl-1H-pyrazol-4-yl)-3-(oxetan-3-yloxy)-9H-pyrrolo[2,3-b:5,4-c′]dipyridin-4-yl]benzamide
N-[(1S,2S)-2-aminocyclopentyl]-4-[3-(2-methoxyethoxy)-6-(1-methyl-1H-pyrazol-4-yl)-9H-pyrrolo[2,3-b:5,4-c′]dipyridin-4-yl]benzamide
N-[(1S,2S)-2-(ethylamino)cyclopentyl]-4-[3-(2-methoxyethoxy)-6-(1-ethyl-1H-pyrazol-4-yl)-9H-pyrrolo[2,3-b:5,4-c′]dipyridin-4-yl]benzamide
4-[6-(1-ethyl-1H-pyrazol-4-yl)-3-methoxy-9H-pyrrolo[2,3-b:5,4-c′]dipyridin-4-yl]-N-[(2R)-pyrrolidin-2-ylmethyl]benzamide
N-[(1S,2S)-2-(cyclopropylamino)cyclohexyl]-4-[3-fluoro-6-(1-methyl-1H-pyrazol-4-yl)-9H-pyrrolo[2,3-b:5,4-c′]dipyridin-4-yl]benzamide
N-[(1S,2S)-2-aminocyclohexyl]-4-[3-methoxy-6-(1-methyl-1H-pyrazol-4-yl)-9H-pyrrolo[2,3-b:5,4-c′]dipyridin-4-yl]benzamide
4-[6-(1-methyl-1H-pyrazol-4-yl)-3-(oxetan-3-yloxy)-9H-pyrrolo[2,3-b:5,4-c′]dipyridin-4-yl]-N-[(2R)-pyrrolidin-2-ylmethyl]benzamide
4-[3-methoxy-6-(1-methyl-1H-pyrazol-4-yl)-9H-pyrrolo[2,3-b:5,4-c′]dipyridin-4-yl]-N-[(2S)-pyrrolidin-2-ylmethyl]benzamide
4-[3-fluoro-6-(pyridin-3-yl)-9H-pyrrolo[2,3-b:5,4-c′]dipyridin-4-yl]-N-{[(2R,3R)-3-hydroxypyrrolidin-2-yl]methyl}benzamide
N-[(3R,4R)-4-aminotetrahydrofuran-3-yl]-4-[3-(2-methoxyethoxy)-6-(pyridin-3-yl)-9H-pyrrolo[2,3-b:5,4-c′]dipyridin-4-yl]benzamide
N-[(1R,2R)-2-(ethylamino)cyclohexyl]-4-[3-methoxy-6-(1-methyl-1H-pyrazol-4-yl)-9H-pyrrolo[2,3-b:5,4-c′]dipyridin-4-yl]benzamide
N-{[(2R,3R,4S)-3,4-dihydroxypyrrolidin-2-yl]methyl}-4-[3-methoxy-6-(1-methyl-1H-pyrazol-4-yl)-9H-pyrrolo[2,3-b:5,4-c′]dipyridin-4-yl]benzamide
N-[(1S,2S)-2-hydroxycyclohexyl]-4-[3-methoxy-6-(1-methyl-1H-pyrazol-4-yl)-9H-pyrrolo[2,3-b:5,4-c′]dipyridin-4-yl]benzamide
N-({4-[3-fluoro-6-(1-methyl-1H-pyrazol-4-yl)-9H-pyrrolo[2,3-b:5,4-c′]dipyridin-4-yl]-phenyl}carbonyl)-β-D-galactopyranosylamine
N-[(3R,4R)-4-aminotetrahydrofuran-3-yl]-4-[3-fluoro-6-(1-methyl-1H-pyrazol-4-yl)-9H-pyrrolo[2,3-b:5,4-c′]dipyridin-4-yl]benzamide
N-[(1S,2R,3S,4S)-2,3-dihydroxy-4-(hydroxymethyl)cyclopentyl]-4-[3-fluoro-6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-[(3S,4R)-4-hydroxytetrahydrofuran-3-yl]benzamide
4-[3-fluoro-6-(1-methyl-1H-pyrazol-4-yl)-9H-pyrrolo[2,3-b:5,4-c′]dipyridin-4-yl]-N-[(1S,2S)-2-hydroxycyclopentyl]benzamide
N-[(1R)-7-azaspiro[3.5]non-1-yl]-4-[3-fluoro-6-(1-methyl-1H-pyrazol-4-yl)-9H-pyrrolo[2,3-b:5,4-c′]dipyridin-4-yl]benzamide
N-[(1S)-7-azaspiro[3.5]non-1-yl]-4-[3-fluoro-6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-[(1S,2S)-2-hydroxycyclohexyl]benzamide
4-[3-fluoro-6-(1-methyl-1H-pyrazol-4-yl)-9H-pyrrolo[2,3-b:5,4-c′]dipyridin-4-yl]-N-[(3S,4S)-4-hydroxypyrrolidin-3-yl]benzamide
4-[3-fluoro-6-(1-methyl-1H-pyrazol-4-yl)-9H-pyrrolo[2,3-b:5,4-c′]dipyridin-4-yl]-N-[(3R,4R)-4-hydroxypyrrolidin-3-yl]benzamide
4-[3-fluoro-6-(1-methyl-1H-pyrazol-4-yl)-9H-pyrrolo[2,3-b:5,4-c′]dipyridin-4-yl]-N-[(3S,4R)-4-hydroxypyrrolidin-3-yl]benzamide
4-[3-fluoro-6-(1-methyl-1H-pyrazol-4-yl)-9H-pyrrolo[2,3-b:5,4-c′]dipyridin-4-yl]-N-[(3R,4S)-4-hydroxypyrrolidin-3-yl]benzamide
4-[3-fluoro-6-(1-methyl-1H-pyrazol-4-yl)-9H-pyrrolo[2,3-b:5,4-c′]dipyridin-4-yl]-N-[(3R,4R)-4-hydroxy-1,1-dioxidotetrahydrothiophen-3-yl]benzamide
4-[3-fluoro-6-(1-methyl-1H-pyrazol-4-yl)-9H-pyrrolo[2,3-b:5,4-c′]dipyridin-4-yl]-N-[(3R,4S)-4-hydroxy-1,1-dioxidotetrahydrothiophen-3-yl]benzamide
4-[3-fluoro-6-(1-methyl-1H-pyrazol-4-yl)-9H-pyrrolo[2,3-b:5,4-c′]dipyridin-4-yl]-N-{2-[(2R)-2-(hydroxymethyl)pyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(2S)-2-(hydroxymethyl)pyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-[(1S,3R)-3-hydroxycyclopentyl]benzamide
4-[3-fluoro-6-(1-methyl-1H-pyrazol-4-yl)-9H-pyrrolo[2,3-b:5,4-c′]dipyridin-4-yl]-N-[(1R,3R)-3-hydroxycyclopentyl]benzamide
4-[3-fluoro-6-(1-methyl-1H-pyrazol-4-yl)-9H-pyrrolo[2,3-b:5,4-c′]dipyridin-4-yl]-N-[(1S,3S)-3-hydroxycyclopentyl]benzamide
4-[3-fluoro-6-(1-methyl-1H-pyrazol-4-yl)-9H-pyrrolo[2,3-b:5,4-c′]dipyridin-4-yl]-N-[(1R,3S)-3-hydroxycyclopentyl]benzamide
4-[3-fluoro-6-(1-methyl-1H-pyrazol-4-yl)-9H-pyrrolo[2,3-b:5,4-c′]dipyridin-4-yl]-N-[(1S,2R)-2-(hydroxymethyl)cyclopentyl]benzamide
4-[3-fluoro-6-(1-methyl-1H-pyrazol-4-yl)-9H-pyrrolo[2,3-b:5,4-c′]dipyridin-4-yl]-N-[(1S,2R)-2-(hydroxymethyl)-1-methylcyclopentyl]benzamide
4-[3-fluoro-6-(1-methyl-1H-pyrazol-4-yl)-9H-pyrrolo[2,3-b:5,4-c′]dipyridin-4-yl]-N-[(2S,3R)-3-(hydroxymethyl)bicyclo[2.2.1]hept-2-yl]benzamide
4-[3-fluoro-6-(1-methyl-1H-pyrazol-4-yl)-9H-pyrrolo[2,3-b:5,4-c′]dipyridin-4-yl]-N-[(2R,3S)-3-(hydroxymethyl)bicyclo[2.2.1]hept-2-yl]benzamide
4-[3-fluoro-6-(1-methyl-1H-pyrazol-4-yl)-9H-pyrrolo[2,3-b:5,4-c′]dipyridin-4-yl]-N-{2-[(2R)-2-(hydroxymethyl)piperidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(2S)-2-(hydroxymethyl)piperidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3S)-3-(hydroxymethyl)piperidin-1-yl]ethyl}benzamide
4-[3-fluoro-6-(1-methyl-1H-pyrazol-4-yl)-9H-pyrrolo[2,3-b:5,4-c′]dipyridin-4-4-yl]-N-{2-[(3R)-3-(hydroxymethyl)piperidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3S)-3-hydroxypiperidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3R)-3-hydroxypiperidin-1-yl]ethyl}benzamide
4-[3-fluoro-6-(1-methyl-1H-pyrazol-4-yl)-9H-pyrrolo[2,3b:5,4-c′]dipyridin-4-yl]-N-[(1S,2S)-2-(hydroxymethyl)cyclohexyl]benzamide
4-[3-fluoro-6-(1-methyl-1H-pyrazol-4-yl)-9H-pyrrolo[2,3-b:5,4-c′]dipyridin-4-yl]-N-[(1R,2R)-2-(hydroxymethyl)cyclohexyl]benzamide
4-[3-fluoro-6-(1-methyl-1H-pyrazol-4-yl)-9H-pyrrolo[2,3-b:5,4-c′]dipyridin-4-yl]-N-[(2R,3R)-3-(hydroxymethyl)-7-oxabicyclo[2.2.1]hept-2-yl]benzamide
N-[(1R,3R)-5-azaspiro[2.4]hept-1-yl]-4-[3-fluoro-6-(1-methyl-1H-pyrazol-4-yl)-9H-pyrrolo[2,3-b:5,4-c′]dipyridin-4-yl]benzamide
N-[(1R,3S)-5-azaspiro[2.4]hept-1-yl]-4-[3-fluoro-6-(1-methyl-1H-pyrazol-4-yl)-9H-pyrrolo[2,3-b:5,4-c′]dipyridin-4-yl]benzamide
N-[(1S,3R)-5-azaspiro[2.4]hept-1-yl]-4-[3-fluoro-6-(1-methyl-1H-pyrazol-4-yl)-9H-pyrrolo[2,3-b:5,4-c′]dipyridin-4-yl]benzamide
N-[(1S,3S)-5-azaspiro[2.4]hept-1-yl]-4-[3-fluoro-6-(1-methyl-1H-pyrazol-4-yl)-9H-pyrrolo[2,3-b:5,4-c′]dipyridin-4-yl]benzamide
N-[(1R,3R)-5-azaspiro[2.5]oct-1-yl]-4-[3-fluoro-6-(1-methyl-1H-pyrazol-4-yl)-9H-pyrrolo[2,3-b:5,4-c′]dipyridin-4-yl]benzamide
N-[(1R,3S)-5-azaspiro[2.5]oct-1-yl]-4-[3-fluoro-6(1-methyl-1H-pyrazol-4-yl)-9H-pyrrolo[2,3-b:5,4-c′]dipyridin-4-yl]benzamide
N-[(1S,3R)-5-azaspiro[2.5]oct-1-yl]-4-[3-fluoro-6-(1-methyl-1H-pyrazol-4-yl)-9H-pyrrolo[2,3-b:5,4-c′]dipyridin-4-yl]benzamide
N-[(1S,3S)-5-azaspiro[2.5]opt-1-yl]-4-[3-fluoro-6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-[(2S)-pyrrolidin-2-ylmethyl]benzamide
4-[3-fluoro-6-(1-methyl-1H-pyrazol-4-yl)-9H-pyrrolo[2,3-b:5,4-c′]dipyridin-4-yl]-N-[(2R)-pyrrolidin-2-ylmethyl]benzamide
4-[3-fluoro-6-(1-methyl-1H-pyrazol-4-yl)-9H-pyrrolo[2,3-b:5,4-c′]dipyridin-4-yl]-N-[(3R)-pyrrolidin-3-ylmethyl]benzamide
4-[3-fluoro-6-(1-methyl-1H-pyrazol-4-yl)-9H-pyrrolo[2,3-b:5,4-c′]dipyridin-4-yl]-N-[(3S)-pyrrolidin-3-ylmethyl]benzamide
4-[3-fluoro-6-(1-methyl-1H-pyrazol-4-yl)-9H-pyrrolo[2,3-b:5,4-c′]dipyridin-4-yl]-N-[(2S)-piperidin-2-ylmethyl]benzamide
4-[3-fluoro-6-(1-methyl-1H-pyrazol-4-yl)-9H-pyrrolo[2,3-b:5,4-c′]dipyridin-4-yl]-N-[(2R)-piperidin-2-ylmethyl]benzamide
4-[3-fluoro-6-(1-methyl-1H-pyrazol-4-yl)-9H-pyrrolo[2,3-b:5,4-c′]dipyridin-4-yl]-N-[(3R)-piperidin-3-ylmethyl]benzamide
4-[3-fluoro-6-(1-methyl-1H-pyrazol-4-yl)-9H-pyrrolo[2,3-b:5,4-c′]dipyridin-4-yl]-N-[(3S)-piperidin-3-ylmethyl]benzamide
3,4-dihydro-1,8-naphthyridin-1(2H)-yl{4-[3-fluoro-6-(1-methyl-1H-pyrazol-4-yl)-9H-pyrrolo[2,3-b:5,4-c′]dipyridin-4-yl]phenyl}methanone
1-({4-[3-fluoro-6-(1-methyl-1H-pyrazol-4-yl)-9H-1-pyrrolo[2,3-b:5,4-c′]dipyridin-4-yl]-phenyl}carbonyl)-2,3-dihydro-1,8-naphthyridin-4(1H)-one
{4-[3-fluoro-6-(1-methyl-1H-pyrazol-4-yl)-9H-pyrrolo[2,3-b:5,4-c′]dipyridin-4-yl]-phenyl}(octahydroquinoxalin-1(2H)-yl)methanone
4-[3-fluoro-6-(1-methyl-1H-pyrazol-4-yl)-9H-pyrrolo[2,3-b:5,4-c′]dipyridin-4-yl]-N-(1,3-thiazol-2-ylmethyl)benzamide
N-[(5-cyano-1,3-thiazol-2-yl)methyl]-4-[3-fluoro-6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-[1-(1,3-thiazol-2-yl)ethyl]benzamide
{4-[3-fluoro-6-(1-methyl-1H-pyrazol-4-yl)-9H-pyrrolo[2,3-b:5,4-c′]dipyridin-4-yl]-phenyl}[2-(1,3-thiazol-2-yl)piperidin-1-yl]methanone
{4-[3-fluoro-6-(1-methyl-1H-pyrazol-4-yl)-9H-pyrrolo[2,3-b:5,4-c′]dipyridin-4-yl]-phenyl}[2-(1,3-thiazol-2-yl)pyrrolidin-1-yl]methanone
[(3S)-3-aminopiperidin-1-yl]{4-[3-methoxy-6-(1-methyl-1H-pyrazol-4-yl)-9H-pyrrolo-[2,3-b:5,4-c′]dipyridin-4-yl]phenyl}methanone
[(3R,4R)-3-amino-4-hydroxypiperidin-1-yl]{4-[3-methoxy-6-(1-methyl-1H-pyrazol-4-yl)-9H-pyrrolo[2,3-b:5,4-c′]dipyridin-4-yl]phenyl}methanone
N-[(3R,4R)-4-hydroxypiperidin-3-yl]-4-[3-methoxy-6-(1-methyl-1H-pyrazol-4-yl)-9H-pyrrolo[2,3-b:5,4-c′]dipyridin-4-yl]benzamide
N-[(3S,4S)-4-hydroxypiperidin-3-yl]-4-[3-methoxy-6-(1-methyl-1H-pyrazol-4-yl)-9H-pyrrolo[2,3-b:5,4-c′]dipyridin-4-yl]benzamide
N-[(3R,4S)-4-hydroxypiperidin-3-yl]-4-[3-methoxy-6-(1-methyl-1H-pyrazol-4-yl)-9H-pyrrolo[2,3-b:5,4-c′]dipyridin-4-yl]benzamide
N-[(3S,4R)-4-hydroxypiperidin-3-yl]-4-[3-methoxy-6-(1-methyl-1H-pyrazol-4-yl)-9H-pyrrolo[2,3-b:5,4-c′]dipyridin-4-yl]benzamide
N-[(3S,5R)-5-hydroxypiperidin-3-yl]-4-[3-methoxy-6-(1-methyl-1H-pyrazol-4-yl)-9H-pyrrolo[2,3-b:5,4-c′]dipyridin-4-yl]benzamide
N-[(1S,2S)-2-aminocyclopentyl]-4-[6-(1-methyl-1H-pyrazol-4-yl)-9H-pyrrolo[2,3-b:5,4-c′]dipyridin-4-yl]benzamide
N-[(1S,2S)-2-(ethylamino)cyclopentyl]-4-[3-methyl-6-(1-methyl-1H-pyrazol-4-yl)-9H-pyrrolo[2,3-b:5,4-c′]dipyridin-4-yl]benzamide
4-[6-(pyridin-3-yl)-9H-pyrrolo[2,3-b:5,4-c′]dipyridin-4-yl]-N-[(2S)-pyrrolidin-2-yl-methyl]benzamide
N-[(1S,2S)-2-aminocyclohexyl]-4-[6-(1-methyl-1H-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-[(3S,4S)-4-hydroxypiperidin-3-yl]benzamide
N-{2-[(3S)-3-hydroxypyrrolidin-1-yl]ethyl}-4-[3-methyl-6-(pyridin-3-yl)-9H-pyrrolo[2,3-b:5,4-c′]dipyridin-4-yl]benzamide
2-(3-aminopropyl)-5-[3-fluoro-6-(1-methyl-1H-pyrazol-4-yl)-9H-pyrrolo[2,3-b:5,4-c′]dipyridin-4-yl]-2,3-dihydro-1H-isoindol-1-one.

The compounds of formula (I) may comprise one or more asymmetric carbons. They may thus exist in the form of enantiomers or diastereoisomers. These enantiomers and diastereoisomers, and also mixtures thereof, including racemic mixtures, form part of the invention.

The compounds of formula (I) may comprise one or more stereochemistries of E/Z type on double bonds or of cis/trans type on non-aromatic rings. These various stereoisomers, and also mixtures thereof, form part of the invention.

The compounds of formula (I) may exist in the form of bases or of acid-addition salts. Such addition salts form part of the invention.

These salts may be prepared with pharmaceutically acceptable acids (P. Stahl, C. Wermuth; Handbook of Pharmaceutical Salts; Wiley Ed.), but the salts of other acids that are useful, for example, for purifying or isolating the compounds of formula (I) also form part of the invention.

The compounds of formula (I) may comprise one or more isotopes of the atoms described above, especially deuterium D, tritium T, ¹¹C, ¹³C, ¹⁴C, ¹⁴C, ¹⁵O, ¹⁵N, ¹⁸F, ¹²³I, ¹²⁴I, and ¹³⁵I. These compounds, irrespective of their isotropic composition, form part of the invention.

All the synthetic intermediates not described in the literature, which lead to the production of the compounds belonging to the general formula, also form part of the invention.

The strategy for the synthesis of the tricyclic nucleus is based on two coupling reactions; a carbon-carbon bond is first created between two suitably selected pyridines, leading to the intermediate of formula Bn, and the formation of an intramolecular carbon-nitrogen bond then leads to the 9H-pyrrolo[2,3-b:5,4-c′]dipyridine unit (intermediate of formula Cn, see scheme 1 below).

A subject of the present invention is also any synthetic process known to those skilled in the art allowing the preparation of the products of formula (I) as defined above.

A subject of the present invention is especially a general process for synthesizing the products of formula (I) as defined above, described hereinbelow in the general scheme:

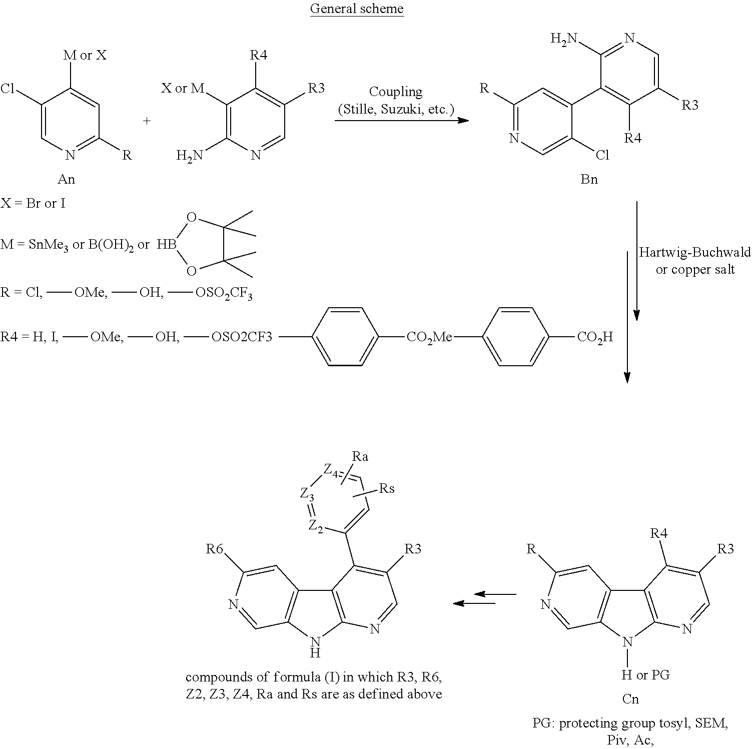

A subject of the present invention is especially a process for synthesizing the products of formula (Ia) as defined above, described hereinbelow in Scheme 1.

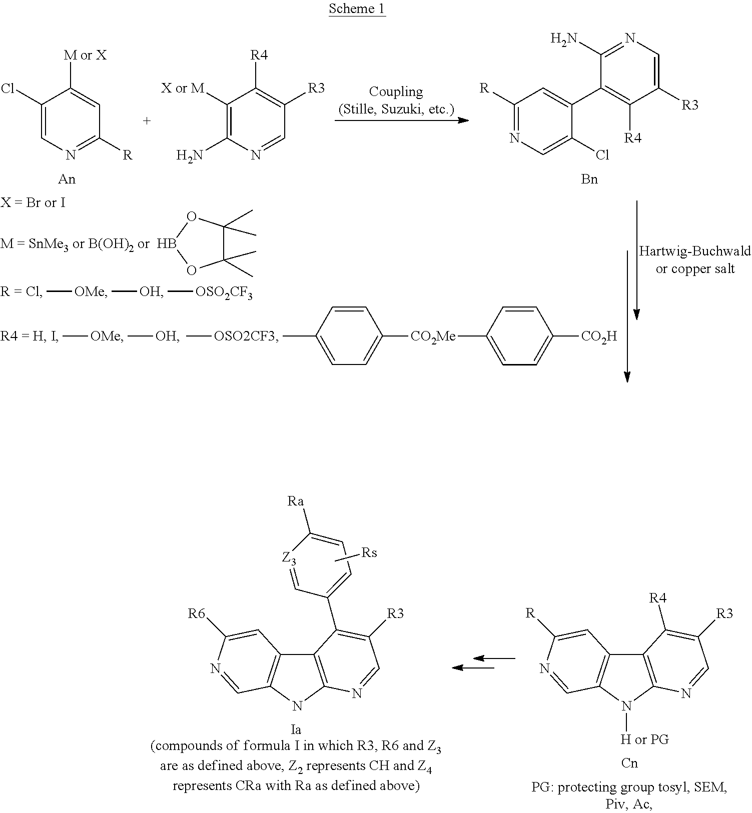

The operating conditions for obtaining the products of formula Ia as defined above, starting from the intermediates of formulae An, Bn and Cn in which X, M, R, R4 and PG have the definitions given in the general scheme as defined above, are described hereinbelow.

For example, the process for preparing the compounds bearing a (3′-pyridinyl) group in position 6 according to the invention consists, in a first step, in preparing 5-chloro-4-(trimethylstannyl)-2,3′-bipyridine A1 from 2-(3′-pyridyl)-5-chloropyridine (Journal of the Chemical Society, Perkin Transactions 1, 2002, (16), 1847-1849) (Scheme 2):

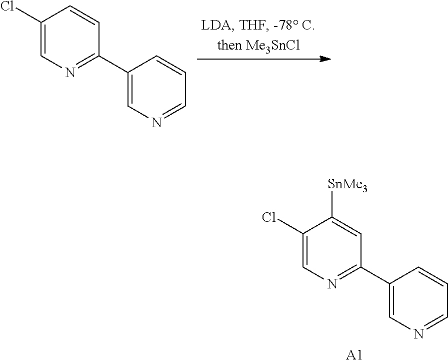

In a second step, Stille coupling is performed with a 2-amino-3-(bromo or iodo)pyridine derivative optionally substituted in position 5 (intermediate B1, Scheme 3):

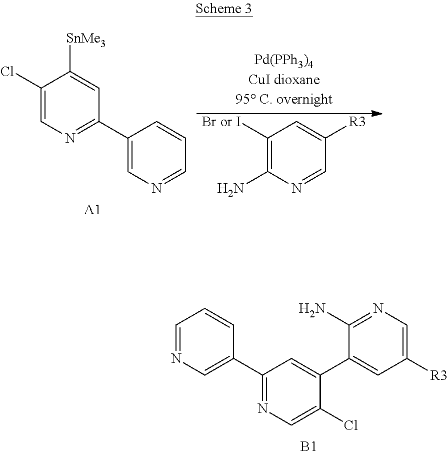

In a third step, the tricyclic unit (intermediate of C1 type) is obtained via an intramolecular aryl amination reaction, catalysed either with a palladium complex, or with copper (I) iodide (Scheme 4):

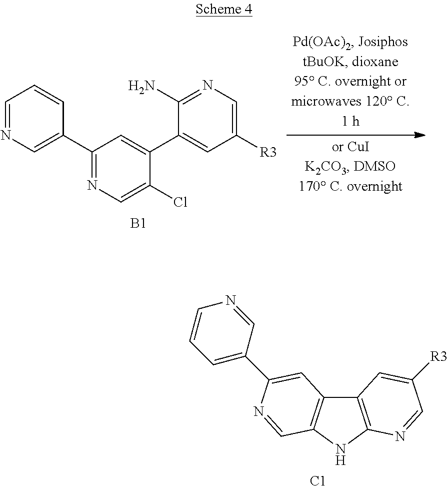

in which Josiphos is a compound having the following formula:

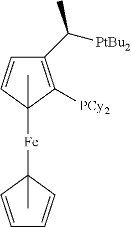

In a fourth step, functionalization of position 4 (C1→C2→C3) is performed (Scheme 5):

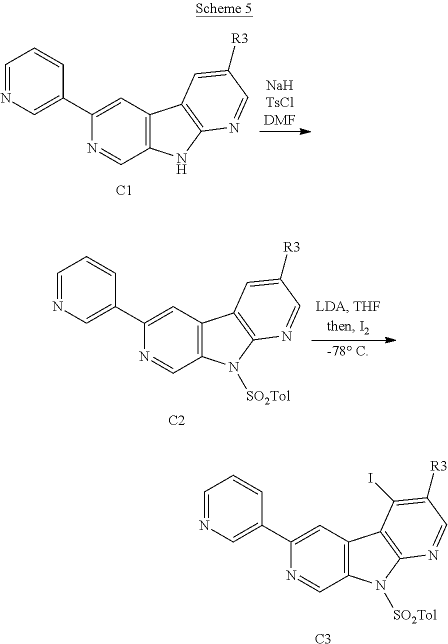

The end of the synthesis is performed in 4 steps: a coupling of Suzuki type (C3→C4), a deprotection via the action of lithium hydroxide (C4→C5), an activation of the acid function with thionyl chloride or with a known carboxylic acid-activating reagent, followed by reaction with the chosen amine (C5→Ib) (Scheme 6):

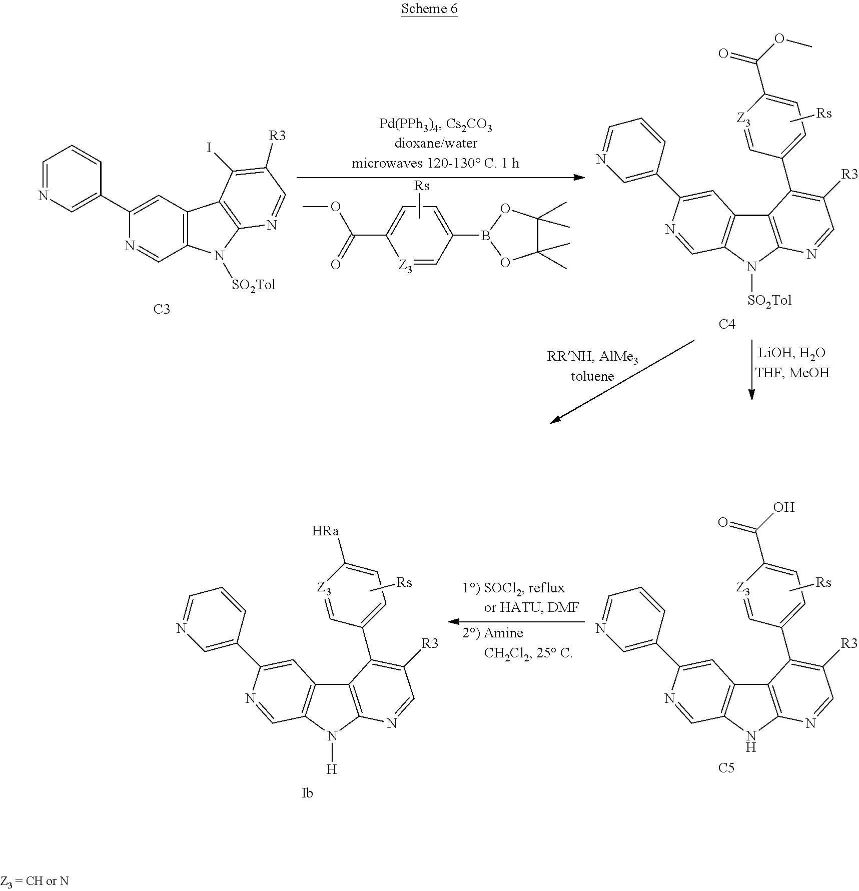

A subject of the present invention is thus especially a process for synthesizing the products of formula (Ib) as defined above, described in Scheme 6.

Depending on the commercial availability of the boronic derivatives, the products of formula of structure Ic may be obtained with a boronic derivative already comprising a carboxamide unit Ra (Scheme 7):

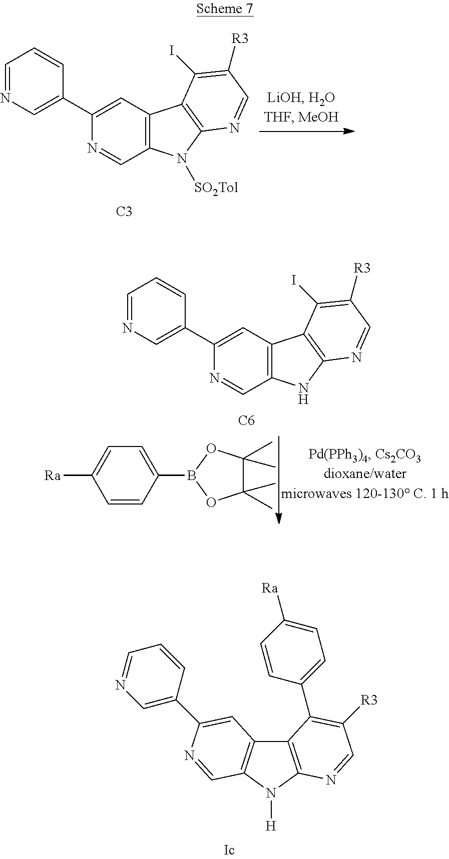

One subject of the present invention is thus especially a process for synthesizing the products of formula (Ic) as defined above, described in Scheme 7.

The boronic reagent, bearing the carboxamido function, may also be prepared before condensation as for a commercial derivative.

The process for preparing the compounds bearing a (1′-methyl-1′H-pyrazol-4′-yl) unit in position 6 according to the invention consists in a first step in preparing the intermediate A2 (Scheme 8):

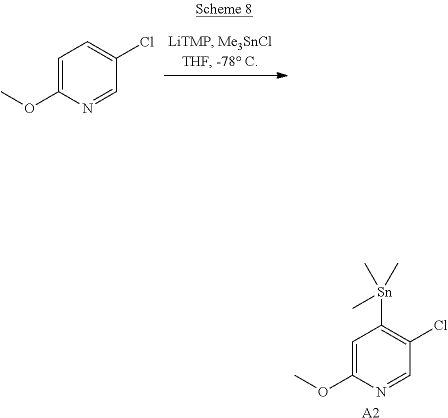

In a second step, Stille coupling is performed (A2→B2) with a 2-amino-3-(bromo or iodo)pyridine derivative optionally substituted in position 4 or 5, followed by an intramolecular aryl amination reaction (B2→C7), catalysed either with a palladium complex, or with copper (I) iodide (Scheme 9):

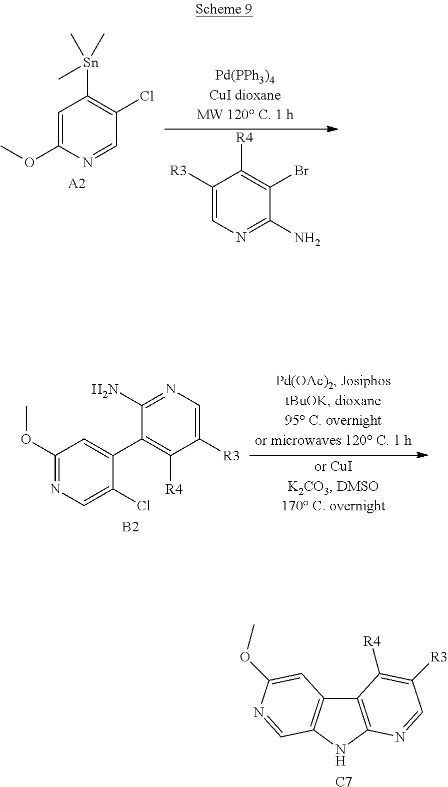

Installation of the (1′-methyl-1′H-pyrazol-4′-yl) unit takes place via a sequence of three steps comprising: a demethylation reaction (C7→C8), the formation of a triflate derivative (C8→C9) and a coupling reaction of Suzuki type (C9→C10) (Scheme 10):

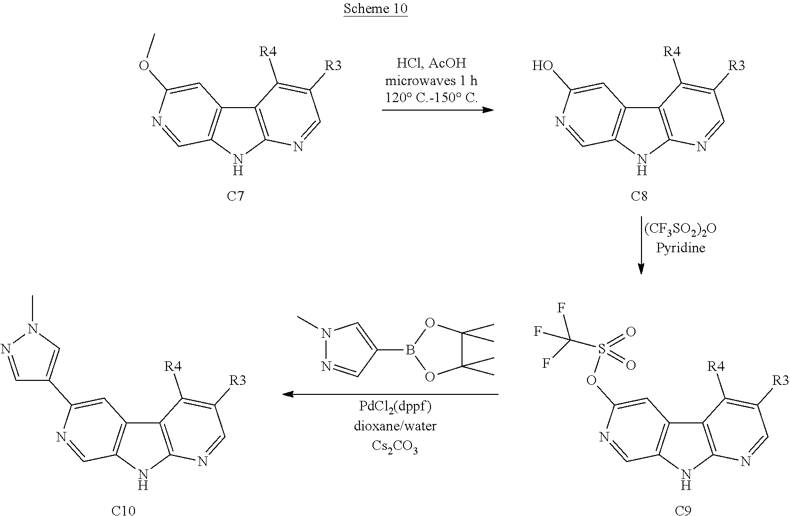

Introduction of the phenylcarboxamido unit takes place as previously via a palladium-catalysed coupling of Suzuki type starting with a triflate or an iodide (intermediate C11, Scheme 11):

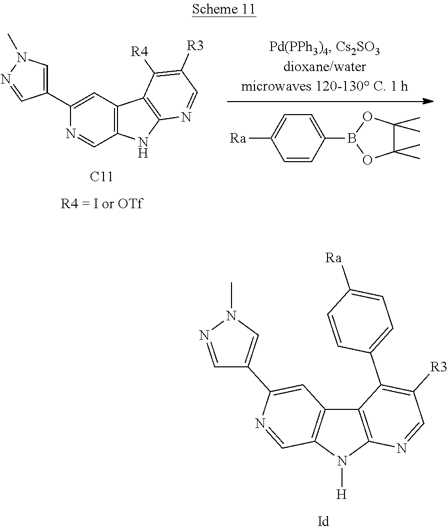

One subject of the present invention is thus especially a process for synthesizing the products of formula (Id) as defined above, described in Scheme 11.

An alternative synthesis for obtaining compounds Id bearing the (1′-methyl-1′H-pyrazol-4′-yl) group was also used. The first step involves preparation of 2,5-dichloro-4-(trimethylstannyl)pyridine A3 via a method analogous to that described hereinabove. The Stille coupling (A3→B3) with a 2-amino-3-(bromo or iodo)pyridine derivative substituted in position 5 is, in this case, followed by an intramolecular aryl amination reaction (B3→C12), catalysed with a copper salt in oxidation state (I) or (II) (Scheme 12):

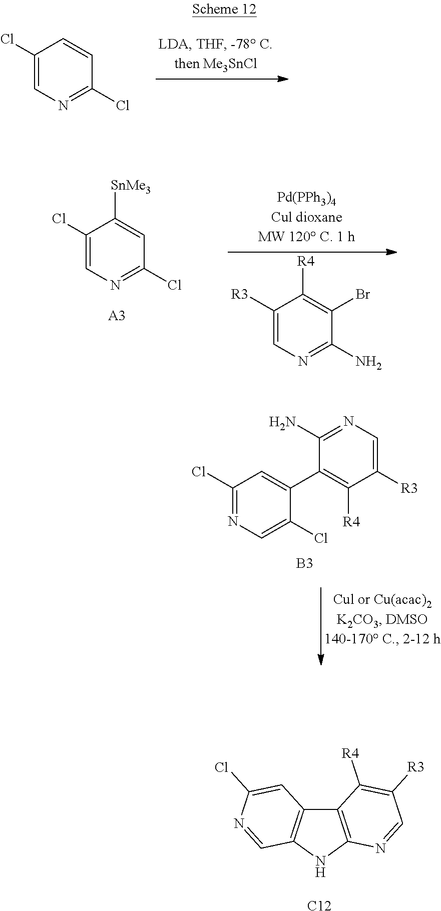

In a second step to be performed, functionalization of position 4 is performed via the same sequence (C1→C2→C3→C6) as for the compounds bearing a 3′-pyridyl group (Scheme 13).

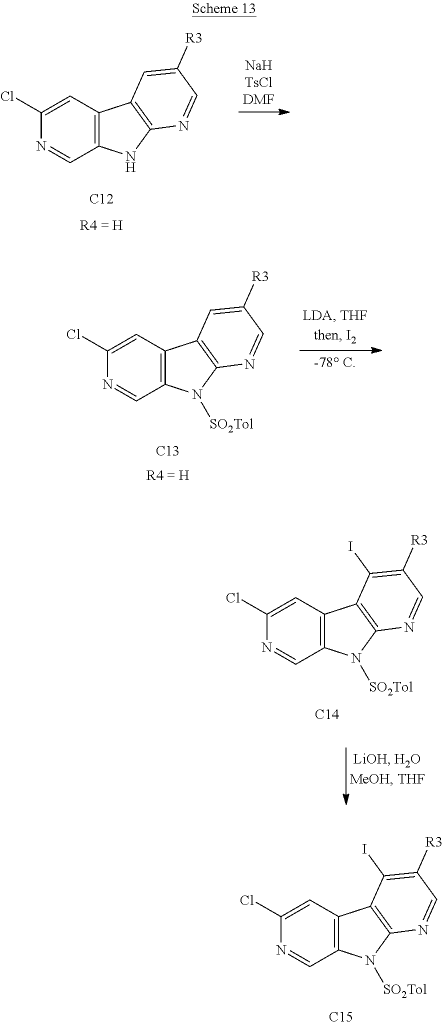

The end of the synthesis first involves two consecutive chemoselective couplings of Suzuki type (C15→C16→C17). After saponification of the ester, the final amide D2 is formed via conventional carboxylic acid-activating methods (Scheme 14). The intermediates of C5 type and the products of formula (I) of formula Ic are also accessible via this synthetic route.

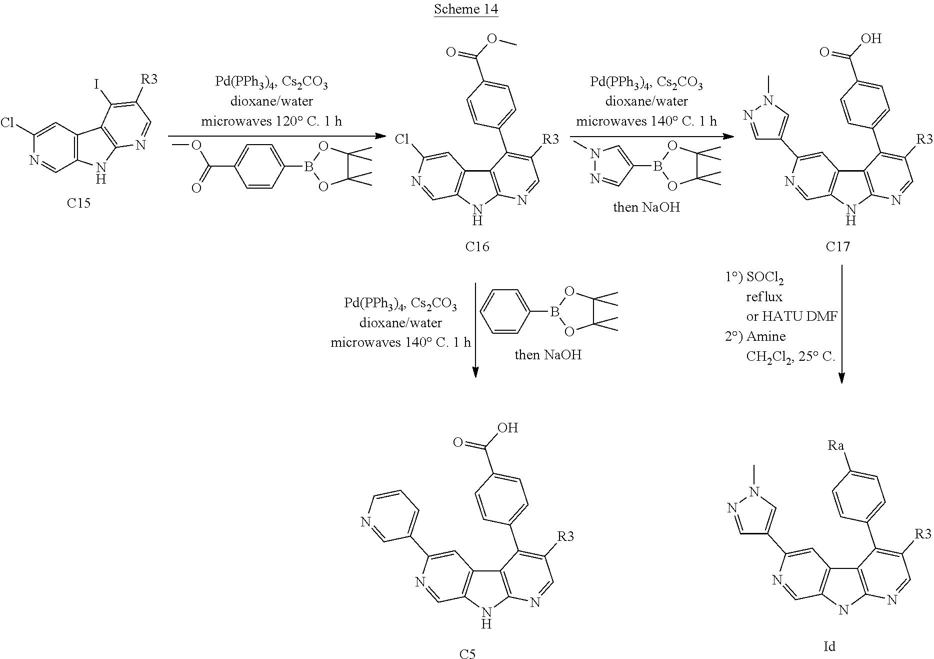

One subject of the present invention is thus especially a process for synthesizing the products of formula (Id) as defined above, described in Scheme 14.

Introduction of an alkoxy group into position 3 (intermediate C18) is performed using the derivative 3-bromo-6-(pyridin-3-yl)-9H-pyrrolo[2,3-b:5,4-c′]dipyridine obtained via the action of dibromine in acetic acid on 6-(pyridin-3-yl)-9H-pyrrolo[2,3-b:5,4-c′]dipyridine. For example, a methoxy or ethoxymethoxy unit may be introduced in the presence of copper (I) iodide (Scheme 15).

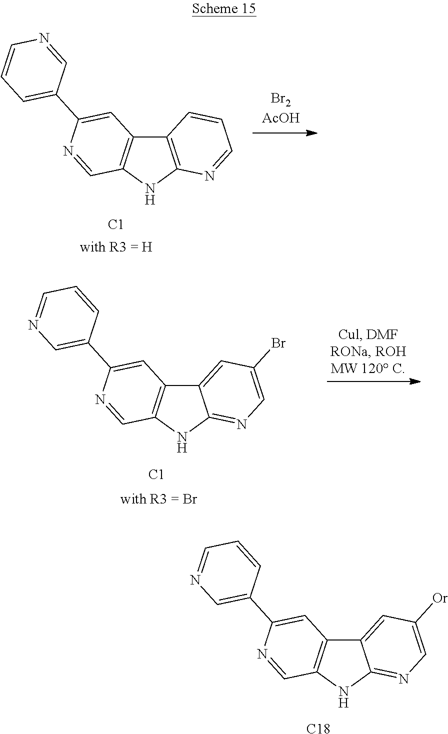

The following steps, namely the introduction of the iodine atom into position 4 and the formation of the phenylcarboxamido chain, are identical to those described hereinabove (C18→C2→C3→C6→Ic with R3=alkoxy).

A subject of the present invention is also, as medicaments, the products of formula (I) as defined above, and also the prodrugs thereof, the said products of formula (I) being in any possible racemic, enantiomeric or diastereoisomeric isomer form, and also the pharmaceutically acceptable addition salts with mineral and organic acids or with mineral and organic bases of the said products of formula (I).

A subject of the present invention is especially, as medicaments, the products of formula (I) as defined above, the names of which are as follows:
4-[3-fluoro-6-(pyridin-3-yl)-9H-pyrrolo[2,3-b:5,4-c′]dipyridin-4-yl]-N-(1H-tetrazol-5-yl-methyl)benzamide
[(3R)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
{4-[3-fluoro-6-(pyridin-3-yl)-9H-pyrrolo[2,3-b:5,4-c′]dipyridin-4-yl]phenyl}[(3aS,6aS)-5-methylhexahydropyrrolo[3,4-b]pyrrol-1(2H)-yl]methanone
N-[2-(acetylamino)ethyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[3-(2-oxopyrrolidin-1-yl)propyl]benzamide
4-[3-fluoro-6-(pyridin-3-yl)-9H-pyrrolo[2,3-b:5,4-c′]dipyridin-4-yl]-N-[2-(phenyl-amino)ethyl]benzamide
N-[(1-ethylpyrrolidin-2-yl)methyl]-4-[3-fluoro-6-(pyridin-3-yl)-9H-pyrrolo[2,3-b:5,4-c′]dipyridin-4-yl]benzamide
N-[3-(dimethylamino)-2,2-dimethylpropyl]-4-[3-fluoro-6-(pyridin-3-yl)-9H-pyrrolo[2,3-b:5,4-c′]dipyridin-4-yl]benzamide
N-{[(2S)-1-ethylpyrrolidin-2-yl]methyl}-4-[3-fluoro-6-(pyridin-3-yl)-9H-pyrrolo[2,3-b:5,4-c′]dipyridin-4-yl]benzamide
N-(1-ethylpiperidin-3-yl)-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(2-methylpiperidin-1-yl)ethyl]benzamide
4-[3-fluoro-6-(pyridin-3-yl)-9H-pyrrolo[2,3-b:5,4-c′]dipyridin-4-yl]-N-(1-methylazetidin-3-yl)benzamide
[3-(dimethylamino)piperidin-1-yl]{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-[2-methyl-2-(pyrrolidin-1-yl)propyl]benzamide
N-[3-(dimethylamino)propyl]-4-[3-fluoro-6-(pyridin-3-yl)-9H-pyrrolo[2,3-b:5,4-c′]dipyridin-4-yl]-N-methylbenzamide
N-[2-(azepan-1-yl)ethyl]-4-[3-fluoro-6-(pyridin-3-yl)-9H-1-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(1-methylpiperidin-4-yl)ethyl]benzamide
4-[3-fluoro-6-(pyridin-3-yl)-9H-pyrrolo[2,3-b:5,4-c′]dipyridin-4-yl]-N-{2-[(methyl-sulfonyl)amino]ethyl}benzamide
4-[3-fluoro-6-(pyridin-3-yl)-9H-pyrrolo[2,3-b:5,4-c′]dipyridin-4-yl]-N-[2-(pyrrolidin-1-yl)propyl]benzamide
4-[3-fluoro-6-(pyridin-3-yl)-9H-pyrrolo[2,3-b:5,4-c′]dipyridin-4-yl]-N-methyl-N-[(1-methylpiperidin-2-yl)methyl]benzamide
4-[3-fluoro-6-(pyridin-3-yl)-9H-pyrrolo[2,3-b:5,4-c′]dipyridin-4-yl]-N-[2-(1-methylpyrrolidin-2-yl)ethyl]benzamide
N-[2-(dipropan-2-ylamino)ethyl]-4-[3-fluoro-6-(pyridin-3-yl)-9H-pyrrolo[2,3-b:5,4-c′]dipyridin-4-yl]benzamide
N-[2-(dimethylamino)ethyl]-N-ethyl-4-[3-fluoro-6-(pyridin-3-yl)-9H-pyrrolo[2,3-b:5,4-c′]dipyridin-4-yl]benzamide
N-[1-(dimethylamino)propan-2-yl]-4-[3-fluoro-6-(pyridin-3-yl)-9H-pyrrolo[2,3-b:5,4-c′]dipyridin-4-yl]benzamide
[(3S)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-methyl-N-(1-methylpyrrolidin-3-yl)benzamide
N-[2-(diethylamino)ethyl]-4-[3-fluoro-6-(pyridin-3-yl)-9H-pyrrolo[2,3-b:5,4-c′]dipyridin-4-yl]-N-methylbenzamide
{4-[3-fluoro-6-(pyridin-3-yl)-9H-pyrrolo[2,3-b:5,4-c′]dipyridin-4-yl]phenyl}[4-(2-methoxyethyl)piperazin-1-yl]methanone
4-[3-fluoro-6-(pyridin-3-yl)-9H-pyrrolo[2,3-b:5,4-c′]dipyridin-4-yl]-N-[(3-methyl-1H-pyrazol-4-yl)methyl]benzamide
{4-[3-fluoro-6-(pyridin-3-yl)-9H-pyrrolo[2,3-b:5,4-c′]dipyridin-4-yl]phenyl}(2-methyl-octahydro-5H-pyrrolo[3,4-c]pyridin-5-yl)methanone
N-[4-(dimethylamino)butyl]-4-[3-fluoro-6-(pyridin-3-yl)-9H-pyrrolo[2,3-b:5,4-c]dipyridin-4-yl]benzamide
4-[3-fluoro-6-(pyridin-3-yl)-9H-pyrrolo[2,3-b:5,4-c′]dipyridin-4-yl]-N-(1H-imidazol-2-ylmethyl)benzamide
{4-[3-fluoro-6-(pyridin-3-yl)-9H-pyrrolo[2,3-b:5,4-c′]dipyridin-4-yl]phenyl}(7-methyl-2,7-diazaspiro[4.4]non-2-yl)methanone
4-[3-fluoro-6-(pyridin-3-yl)-9H-pyrrolo[2,3-b:5,4-c′]dipyridin-4-yl]-N-[2-(pyridin-2-yl-amino)ethyl]benzamide
N-ethyl-4-[3-fluoro-6-(pyridin-3-yl)-9H-pyrrolo[2,3-b:5,4-c′]dipyridin-4-yl]-N-[(1-methylpyrrolidin-3-yl)methyl]benzamide
1,3′-bipyrrolidin-1′-yl{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-methyl-N-(1-methylpiperidin-4-yl)benzamide
4-[3-fluoro-6-(pyridin-3-yl)-9H-pyrrolo[2,3-b:5,4-c′]dipyridin-4-yl]-N-[(2-hydroxypyridin-4-yl)methyl]benzamide
N-[2-(ethylamino)ethyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-[2-(methyl-amino)ethyl]benzamide
N-[(1-aminocyclopropyl)methyl]-4-[3-fluoro-6-(pyridin-3-yl)-9H-pyrrolo[2,3-b:5,4-c′]dipyridin-4-yl]benzamide
N-(3-amino-2,2-difluoropropyl)-4-[3-fluoro-6-(pyridin-3-yl)-9H-pyrrolo[2,3-b:5,4-c′]dipyridin-4-yl]benzamide
N-(2-amino-3,3,3-trifluoro-2-methylpropyl)-4-[3-fluoro-6-(pyridin-3-yl)-9H-pyrrolo-[2,3-b:5,4-c′]dipyridin-4-yl]benzamide
N-[(1R,2R)-2-aminocyclohexyl]-4-[3-fluoro-6-(pyridin-3-yl)-9H-pyrrolo[2,3-b:5,4-c′]dipyridin-4-yl]benzamide
N-[(1S,2S)-2-aminocyclohexyl]-4-[3-fluoro-6-(pyridin-3-yl)-9H-pyrrolo[2,3-b:5,4-c]dipyridin-4-yl]benzamide
N-[(1S,2S)-2-aminocyclopentyl]-4-[3-fluoro-6-(pyridin-3-yl)-9H-1-pyrrolo[2,3-b:5,4-c′]dipyridin-4-yl]benzamide
N-[(1R,2R)-2-aminocyclopentyl]-4-[3-fluoro-6-(pyridin-3-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]-N-(4-methylpiperazin-1-yl)benzamide
4-[3-fluoro-6-(1-methyl-1H-pyrazol-4-yl)-9H-pyrrolo[2,3-b:5,4-c′]dipyridin-4-yl]-N-(1-methylpiperidin-4-yl)benzamide
4-[3-fluoro-6-(1-methyl-1H-pyrazol-4-yl)-9H-pyrrolo[2,3-b:5,4-c′]dipyridin-4-yl]-N-{2-[(3R)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3S)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-(2-hydroxyethyl)benzamide
N-[(1S,2S)-2-aminocyclohexyl]-4-[3-fluoro-6-(1-methyl-1H-pyrazol-4-yl)-9H-pyrrolo-[2,3-b:5,4-c′]dipyridin-4-yl]benzamide
N-[(1S,2S)-2-(diethylamino)cyclohexyl]-4-[3-fluoro-6-(1-methyl-1H-pyrazol-4-yl)-9H-pyrrolo[2,3-b:5,4-c′]dipyridin-4-yl]benzamide
N-[(1S,2S)-2-(ethylamino)cyclohexyl]-4-[3-fluoro-6-(1-methyl-1H-yl)-pyrazol-4-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]benzamide
4-[6-(5-chloro-1-methyl-1H-pyrazol-4-yl)-3-fluoro-9H-pyrrolo[2,3-b:5,4-c′]dipyridin-4-yl]-N-(4-methylpiperazin-1-yl)benzamide
4-[6-(5-chloro-1-methyl-1H-pyrazol-4-yl)-3-fluoro-9H-pyrrolo[2,3-b:5,4-c′]dipyridin-4-yl]-N-(1-methylpiperidin-4-yl)benzamide
N-[2-(dimethylamino)ethyl]-4-[3-(2-methoxyethoxy)-6-(pyridin-3-yl)-9H-pyrrolo[2,3-b:5,4-c′]dipyridin-4-yl]benzamide
N-[2-(dimethylamino)ethyl]-5-[3-fluoro-6-(pyridin-3-yl)-9H-pyrrolo[2,3-b:5,4-c′]dipyridin-4-yl]pyridine-2-carboxamide
N-[2-(dimethylamino)ethyl]-2-fluoro-4-[3-fluoro-6-(pyridin-3-yl)-9H-pyrrolo[2,3-b:5,4-c′]dipyridin-4-yl]benzamide
   the said products of formula (I) being in any possible racemic, enantiomeric or diastereoisomeric isomer form, and also the pharmaceutically acceptable addition salts with mineral and organic acids or with mineral and organic bases of the said products of formula (I).

A subject of the present invention is also pharmaceutical compositions containing, as active principle, a compound according to any one of the preceding claims and also at least one pharmaceutically compatible excipient.

A subject of the present invention is also the pharmaceutical compositions according to the preceding claim, used for treating cancer.

Thus, according to another of its aspects, the present invention relates to pharmaceutical compositions comprising, as active principle, a compound according to the invention. These pharmaceutical compositions contain an effective dose of at least one compound according to the invention, or a pharmaceutically acceptable salt of the said compound, and also at least one pharmaceutically acceptable excipient.

The said excipients are chosen, according to the pharmaceutical form and the desired mode of administration, from the usual excipients known to those skilled in the art.

In the pharmaceutical compositions of the present invention for oral, sublingual, subcutaneous, intramuscular or intravenous administration, the active principle of formula (I) above, or its salt, may be administered in unit administration form, as a mixture with standard pharmaceutical excipients, to man and animals for the treatment of the above disorders or diseases.

The appropriate unit administration forms include oral-route forms such as tablets, soft or hard gel capsules, powders, granules and oral solutions or suspensions, and sublingual, buccal, subcutaneous, intramuscular or intravenous administration forms.

These medicaments find their use in therapeutics, especially in the treatment of cancers sensitive to deregulation of the Pim kinases.

The Pim kinase inhibitors that are subjects of the present invention are useful for treating cancer. Insofar as cancer remains a disease for which the existing treatments are insufficient, it is manifestly necessary to identify novel Pim kinase inhibitors that are effective for treating cancer.

According to another of its aspects, the present invention also relates to a method for treating the pathologies indicated above, which comprises the administration, to a patient, of an effective dose of a compound according to the invention, or a pharmaceutically acceptable salt thereof.

The examples that follow describe the preparation of certain compounds in accordance with the invention. These examples are not limiting, and serve merely to illustrate the present invention. The numbers of the presented compounds refer to those given in the table hereinbelow, which illustrates the chemical structures and the physical properties of a number of compounds according to the invention.

Among the examples of preparation of the products of formula I as defined above, certain examples constitute products of formula I as defined above or synthetic intermediates for obtaining the said products of formula I, as defined below:

Examples 7, 9, 10, 11, 12, 13, 14, 15, 16, 17, 19, 20, 21, 22, 23, 24, 25, 26, 27, 28, 37, 38, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 99, 100, 101, 102, 103, 104, 105, 106, 107, 108, 109, 112, 113 and 120 constitute the products of formula I.

Compounds A1, A2 and A3 constitute synthetic intermediates of formula An

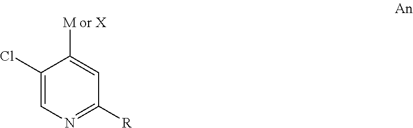

in which X, M and R have the definitions indicated in the general scheme.

Examples 1, 29, 39, 92 and 114 constitute synthetic intermediates of formula Bn

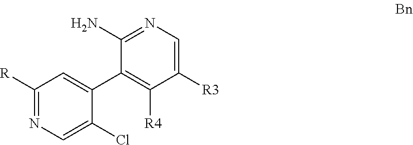

in which R and R4 have the definitions indicated in the general scheme.

Examples 2, 3, 4, 5, 6, 8, 18, 30, 31, 32, 33, 34, 35, 36, 40, 41, 42, 43, 93, 94, 95, 96, 97, 98, 110, 111, 115, 116, 117, 118 and 119 constitute synthetic intermediates of formula Cn

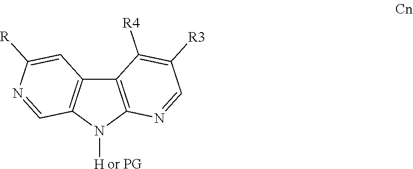

in which R, R3, R4 and PG have the definitions indicated in the general scheme.

Thus X, M, R, R3, R4 and PG represent the definitions indicated in the preparation of the examples according to the present invention and especially hereinbelow:

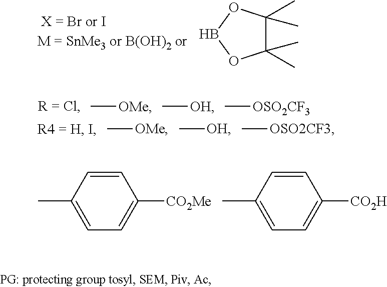

One subject of the present invention is thus especially, as novel industrial products, the synthetic intermediates of the products of formula (I) described in the general scheme and in Schemes 1 to 15 above.

One subject of the present invention is thus, as novel industrial products, the synthetic intermediates of formulae An, Bn and On as defined in the general scheme hereinabove and hereinbelow.

One subject of the present invention is thus especially, as novel industrial products, the synthetic intermediates of the products of formula An described in the scheme above.

One subject of the present invention is thus especially, as novel industrial products, the synthetic intermediates of the products of formula Bn described in the scheme above.

One subject of the present invention is thus especially, as novel industrial products, the synthetic intermediates of the products of formula Cn described in the scheme above.

### General Information

### Abbreviations:

¹H NMR: proton nuclear magnetic resonance
DAD: wavelength-scanning detector
DCM: dichloromethane
DME: 1,2-dimethoxyethane
DMF: dimethylformamide
DMSO: dimethyl sulfoxide
ELSD: light-scattering detector
HATU: O-(7-azabenzotriazol-1-yl)-N,N,N′,N′-tetramethyluronium hexafluorophosphate
HPLC, UPLC: high-performance liquid chromatography
LC: liquid chromatography
LDA: lithium diisopropylamide
LiTMP: lithium amide of 2,2,6,6-tetramethylpiperidine
MS: mass spectrometry
THF: tetrahydrofuran
Tr: retention time

[symbol] All the reactions are performed with anhydrous solvents from the Acros Organics AcroSeal range. The solvents used for the extractions and the chromatographies are obtained from SDS. The microwave reactions are performed in a Biotage or CEM machine. The purifications on silica gel are performed using VWR-Merck silica cartridges (silica gel 60 15-40 μm). The preparative HPLC purifications are performed on Macherey-Nagel columns (Nucleodur C18 phase) or other phases (Chiralcel OD-I or OJ-H or AS-H, Chiralpak, Kromasil C₁₈) with suitable eluents.

[symbol] LC-MS-DAD-ELSD analysis: 2 possible experimental conditions:

[symbol] LC-MS-DAD-ELSD analysis: MS=Waters ZQ; electrospray mode+/−; mass range m/z=100-1200; LC=Agilent HP 1100; LC column=XBridge 18 C Waters 3.0×50 mm-2.5 μm; LC oven=60° C.; flow rate=1.1 ml/min.

Eluents: A=water+0.1% formic acid; B=acetonitrile, with the following gradient:

| Time | A % | B % |
|---|---|---|
| 0.0 | 95 | 5 |
| 5.0 | 0 | 100 |
| 5.5 | 0 | 100 |
| 6.5 | 95 | 5 |
| 7.0 | 95 | 5 |

[symbol] LC-MS-DAD-ELSD analysis: MS=Platform II Waters Micromass; electrospray+/−; mass range m/z=100-1100; LC Alliance 2695 Waters; XTerra 18C Waters column 4.6 mm×75 mm-2.5 μm; LC oven=60° C.; flow rate=1.0 ml/min.

Eluents: A=water+0.1% formic acid; B=acetonitrile, with the following gradient:

| Time | A % | B % |
|---|---|---|
| 0 | 95 | 5 |
| 6.0 | 5 | 95 |
| 8.0 | 5 | 95 |
| 9.0 | 95 | 5 |
| 13.0 | 95 | 5 |

[symbol] HPLC-MS-DAD-ELSD analysis: 2 possible experimental conditions:

[symbol] HPLC-MS-DAD-ELSD analysis: MS=Quattro Premier XE Waters; electrospray+/−; mass range m/z=100-1100; UPLC Waters; Acquity UPLC BeH C18 column 2.1 mm×50 mm-1.7 μm; UPLC oven=70° C.; flow rate=0.7 ml/min.

Eluents: A=water+0.1% formic acid; B=acetonitrile+0.1% formic acid, with the gradient:

| Time | A % | B % |
|---|---|---|
| 0 | 95 | 5 |
| 5 | 0 | 100 |
| 5.5 | 95 | 5 |
| 6.0 | 95 | 5 |

[symbol] HPLC-MS-DAD-ELSD analysis: MS=SQD Waters; electrospray+/−; mass range m/z=100-1100; UPLC-Waters; Acquity UPLC Beh C18 column 2.1 mm×50 mm-1.7 μm; UPLC oven=70° C.; flow rate=1 ml/min.

Eluents: A=water+0.1% formic acid; B=acetonitrile+0.1% formic acid, with the gradient;

| Time | A % | B % |
|---|---|---|
| 0 | 95 | 5 |
| 0.8 | 50 | 50 |
| 1.2 | 0 | 100 |
| 1.85 | 0 | 100 |
| 1.95 | 95 | 5 |
| 2.00 | 95 | 5 |

For the detection:

DAD wavelength considered λ=210-400 nm
ELSD: Sedere SEDEX 85; nebulization temperature=35° C.; nebulization pressure=3.7 bar
N.B: Depending on the structures analysed, the dilution solvents are: dimethyl sulfoxide; methanol; acetonitrile; dichloromethane.

### EXAMPLES

### 5-chloro-4-(trimethylstannyl)-2,3′-bipyridine A1

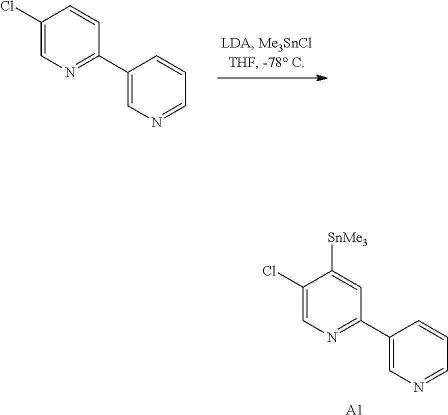

A mixture of 15 ml of diisopropylamine and 40 ml of tetrahydrofuran is cooled to −74° C., followed by addition of 64 ml of 1.6 N n-butyllithium in hexane over 20 minutes, while keeping the temperature below −70° C., 16.2 g of 2-(3′-pyridyl)-5-chloropyridine prepared according to the reference Journal of the Chemical Society, Perkin Transactions 1, 2002, 16, 1847-1849, dissolved in 170 ml of tetrahydrofuran, are added to the reaction mixture, while still keeping the temperature below −70° C. The mixture is stirred at −74° C. for 1 hour 30 minutes, followed by gradual addition of 19.47 g of trimethyltin chloride dissolved in 100 ml of tetrahydrofuran, while keeping the temperature below −70° C. The reaction mixture is stirred for a further 1 hour at a temperature below −72° C., followed by addition of 100 ml of water. The mixture, warmed to room temperature, is poured into 300 ml of water and 100 ml of saturated aqueous potassium hydrogen carbonate solution and than extracted with twice 400 ml of ethyl acetate. The combined organic phases are concentrated to dryness under reduced pressure and the residue is then purified on a Varian SCX cartridge, eluting with pure methanol and then with 2 N ammoniacal methanol, followed by chromatography on a column of silica, eluting with a 100/0 to 50/50 heptane/ethyl acetate mixture to give 19.22 g of 5-chloro-4-(trimethylstannyl)-2,3′-bipyridine A1 in the form of a beige-coloured powder.

UPLC-MS-DAD-ELSD: Tr (min)=4.29; [M+H]⁺: m/z 355; purity: 98%

1H NMR (400 MHz, DMSO-d6): ppm 0.45 (s, 9H) 7.52 (dd. J=7.6, 51 Hz, 1H) 7.94 (d, J=1.0 Hz, 1H) 8.40 (dt, J=7.9, 2.1 Hz, 1H) 8.63 (s, 1H) 8.64-8.66 (m, 1H) 9.22 (d, J=2.4 Hz, 1H)

### Example 1

### 5′-chloro-5″-fluoro-3,2′:4′,3″-terpyridin-2″-amine

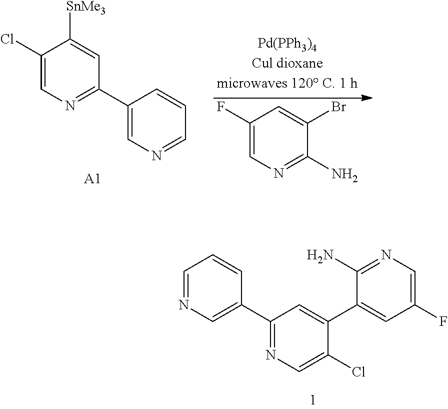

A mixture of 10 g of 2-amino-3-bromo-5-fluoropyridine, 19.2 g of 5-chloro-4-(trimethylstannyl)-2,3′-bipyridine A1, 4.24 g of tetrakis(triphenylphosphine)-palladium(0) and 2.095 g of copper (I) iodide in 120 ml of 1,4-dioxane is refluxed for 18 hours. The reaction medium is treated with aqueous 10% sodium hydrogen carbonate solution and then diluted with ethyl acetate. After separation of the phases by settling, the aqueous phase is extracted twice with ethyl acetate. The combined organic phases are dried over anhydrous sodium sulfate, filtered and concentrated under reduced pressure. The residue is taken up in a mixture of dichloromethane and methanol, and is then filtered by suction to give 11.67 g of 5′-chloro-5″-fluoro-3,2′:4′,3″-terpyridin-2″-amine in the form of a beige-coloured solid. The filtrate is concentrated under reduced pressure and then taken up in dichloromethane, and silica is added. After concentrating under reduced pressure, the deposit is purified by chromatography on a column of silica, eluting with a 98/2 to 90/10 dichloromethane/methanol mixture to give 1.98 g of 5′-chloro-5″-fluoro-3,2′:4′,3″-terpyridin-2″-amine in the form of a beige-coloured solid.

UPLC-MS-DAD-ELSD: Tr (min)=2.71; [M+H]⁺: m/z 301; purity: 95%

1H NMR (400 MHz, DMSO-d6): ppm: 5.78 (s, 2H) 7.47 (dd, J=8.8, 2.9 Hz, 1H) 7.55 (br. s., 1H) 8.06 (s, 1H) 8.12 (s, 1H) 8.49 (d, J=7.8 Hz, 1H) 8.68 (br, s., 1H) 8.84 (s, 1H) 9.35 (br. s., 1H)

### Example 2

### 3-fluoro-6-(pyridin-3-yl)-9H-pyrrolo[2,3-b:5,4-c′]dipyridine

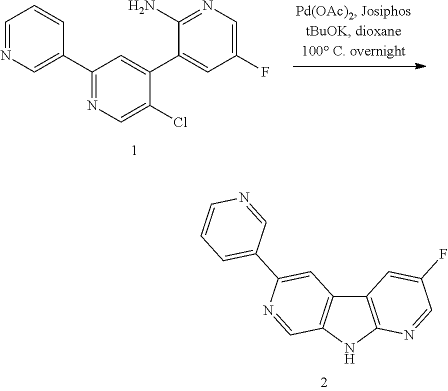

1.574 g of (R)-(−)-1-[(S)-2-(dicyclohexylphosphino)ferrocenyl]ethyldi-tert-butylphosphine and 0.558 g of palladium (II) acetate in 40 ml of anhydrous 1,4-dioxane are placed in a 100 ml reactor under an argon atmosphere and stirred for 10 minutes at 40° C.

11.6 g of 5′-chloro-5″-fluoro-3,2′:4′,3″-terpyridin-2″-amine in 160 ml of anhydrous 1,4-dioxane are placed in a 500 ml reactor under argon and the solution prepared previously is added, followed by addition of 5.97 g of potassium hart-butoxide. The reaction mixture is refluxed for 18 hours. The mixture is diluted with a 71/29 dichloromethane/methanol mixture and is than filtered under vacuum. After concentrating under reduced pressure, the deposit is purified by chromatography on a column of silica, eluting with a 98/2 to 92/8 dichloromethane/methanol mixture to give 6.5 g of 3-fluoro-6-(pyridin-3-yl)-9H-pyrrolo[2,3-b:5,4-c′]dipyridine in the form of a brown solid.

UPLC-MS-DAD-ELSD: Tr (min)=0.42; [M+H]⁺: m/z 265; [M−H]⁺: m/z 263; purity: 98%

1H NMR (400 MHz, DMSO-d6): ppm 7.54 (ddd, J=8.0, 4.7, 0.7 Hz, 1H) 8.48 (dt, J=7.8, 2.0 Hz, 1H) 8.60 (dd, J=4.6, 1.5 Hz, 1H) 8.65 (s, 1H) 8.65-8.68 (m, 1H) 8.90 (d, J=1.2 Hz, 1H) 9.05 (d, J=1.2 Hz, 1H) 9.34 (d, J=1.5 Hz, 1H) 12.39 (br. s., 1H)

### Example 3

### 3-fluoro-9-[(4-methylphenyl)sulfonyl]-6-(pyridin-3-yl)-9H-pyrrolo-[2,3-b:5,4-c′]dipyridine

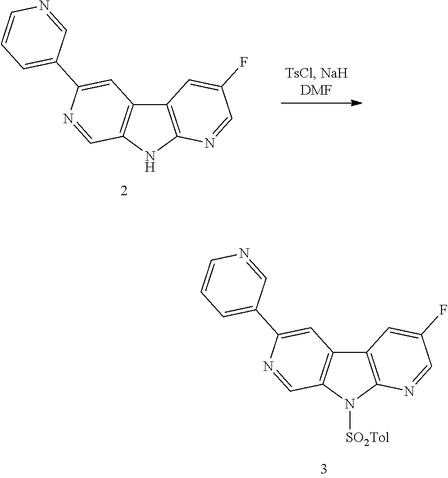

3.20 g of 3-fluoro-6-(pyridin-3-yl)-9H-pyrrolo[2,3-b:5,4-c′]dipyridine in 80 ml of dimethylformamide and 0.847 g of 60% sodium hydride in oil are placed in a 250 ml reactor under argon. After stirring for 3 hours at room temperature, 4.61 g of para-toluenesulfonyl chloride dissolved in 20 ml of dimethylformamide are added. The reaction medium is stirred for 3 hours at room temperature and then treated with aqueous 10% sodium hydrogen carbonate solution and diluted with ethyl acetate. After separation of the phases by settling, the aqueous phase is extracted twice with ethyl acetate. The combined organic phases are washed with water and then dried over anhydrous magnesium sulfate, filtered and concentrated under reduced pressure. The residue is purified by chromatography on a column of silica, eluting with a 100/0 to 95/5 dichloromethane/methanol mixture to give 4.75 g of 3-fluoro-9-[(4-methylphenyl)sulfonyl]-6-(pyridin-3-yl)-9H-pyrrolo[2,3-b:5,4-c′]dipyridine.

UPLC-MS-DAD-ELSD: Tr (min)=0.90; [M+H]⁺: m/z 419; purity: 98%

1H NMR (400 MHz, DMSO-d6) ppm: 2.32 (s, 3H) 7.39 (d, J=81 Hz, 2H) 7.59 (dd, J=8.1, 4.6 Hz, 1H) 8.03 (d, J=8.6 Hz, 2H) 8.51 (dt, J=8.0, 2.0 Hz, 1H) 8.67 (dd, J=4.8, 1.6 Hz, 1H) 8.73 (dd, J=8.3, 2.9 Hz, 1H) 8.78 (dd, J=2.9, 1.2 Hz, 1H) 8.98 (d, J=0.5 Hz, 1H) 9.36 (d, J=1.7 Hz, 1H) 9.70 (d, J=0.7 Hz, 1H)

### Example 4

### 3-fluoro-9-[(4-methylphenyl)sulfonyl]-6-(pyridin-3-yl)-9H-pyrrolo-[2,3-b:5,4-c′]dipyridine

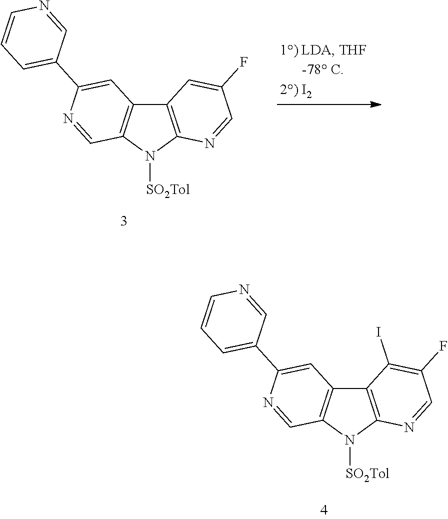

0.43 ml of diisopropylamine in 10 ml of tetrahydrofuran is placed in a round-bottomed flask. After stirring and cooling to −78° C., 1.15 ml of 2.5 N n-butyllithium in hexane are added. The reaction mixture is stirred for 15 minutes at −78° C., followed by addition of 0.800 g of 3-fluoro-9-[(4-methylphenyl)sulfonyl]-6-(pyridin-3-yl)-9H-pyrrolo[2,3-b:5,4-c′]dipyridine in 50 ml of tetrahydrofuran. After stirring for 2 hours at −78° C., 0.776 g of iodine in 5 ml of tetrahydrofuran is added. After stirring for 1 hour, the reaction mixture is poured into 150 ml of aqueous 10% ammonium chloride solution and 50 ml of water, and extracted twice with 150 ml of ethyl acetate. The organic phases are washed with aqueous 5% sodium thiosulfate solution, dried over sodium sulfate, filtered and concentrated to dryness under reduced pressure. 1.03 g of 3-fluoro-4-iodo-9-[(4-methylphenyl)sulfonyl]-6-(pyridin-3-yl)-9H-pyrrolo[2,3-b:5,4-c′]dipyridine are obtained.

UPLC-MS-DAD-ELSD: Tr (min)=1.05; [M+H]⁺: m/z 545; purity: 66%

1H NMR (400 MHz, DMSO-d6) ppm: 2.33 (s, 3H) 7.40 (d, J=81 Hz, 2H) 7.60 (dd, J=7.8, 4.6 Hz, 1H) 8.05 (d, J=8.3 Hz, 2H) 8.47 (dt, J=7.9, 2.0 Hz, 1H) 8.65 (s, 1H) 8.69 (dd, J=4.8, 1.6 Hz, 1H) 9.19 (s, 1H) 9.29 (d, J=2.0 Hz, 1H) 9.79 (s, 1H)

### Example 5

### methyl 4-{3-fluoro-9-[(4-methylphenyl)sulfonyl]-6-(pyridin-3-yl)-9H-pyrrolo[2,3-b:5,4-c′]dipyridin-4-yl}benzoate

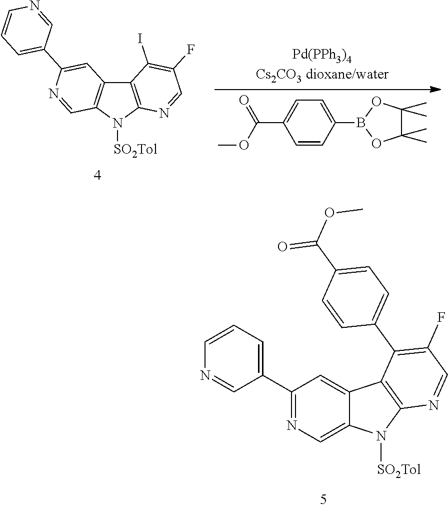

800 mg of 3-fluoro-4-iodo-9-[(4-methylphenyl)sulfonyl]-6-(pyridin-3-yl)-9H-pyrrolo-[2,3-b:5,4-c′]dipyridine, 847 mg of 4-methoxycarbonylphenylboronic acid pinacol ester, 170 mg of tetrakis(triphenylphosphine)palladium(0), 718 mg of caesium carbonate in 12 ml of 1,4-dioxane and 3 ml of water are placed in a reactor, and the tube is sealed and subjected to microwave irradiation at 120° C. for 1 hour. 3 ml of methanol and 100 ml of water are added to the reaction medium and the resulting mixture is then extracted with five times 150 ml of ethyl acetate. The organic phases are combined, dried over sodium sulfate, filtered and concentrated to dryness under reduced pressure. The residue is purified by chromatography on a column of silica, eluting with a 100/0 to 97/3 dichloromethane/methanol mixture to give 793 mg of methyl 4-{3-fluoro-9-[(4-methylphenyl)sulfonyl]-6-(pyridin-3-yl)-9H-pyrrolo[2,3-b:5,4-c′]dipyridin-4-yl}benzoate.

UPLC-MS-DAD-ELSD: Tr (min)=1.10; [M+H]³⁰ : m/z 553; purity: 40%

1H NMR (400 MHz, DMSO-d6) ppm: 2.35 (s, 3H) 3.95 (s, 3H) 7.44 (d, J=7.8 Hz, 2H) 7.47-7.52 (m, 2H) 7.87 (d, J=7.8 Hz, 2H) 8.12 (d, J=8.3 Hz, 2H) 8.13-8.17 (m, 1H) 8.26 (d, J=8.3 Hz, 2H) 8.59 (dd, J=4.9, 1.5 Hz, 1H) 8.86 (d, J=2.4 Hz, 1H) 8.89 (d, J=1.5 Hz, 1H) 9.77 (s, 1H)

### Example 6

### 4-[3-fluoro-6-(pyridin-3-yl)-9H-pyrrolo[2,3-b:5,4-c′]dipyridin-4-yl]-benzoic acid

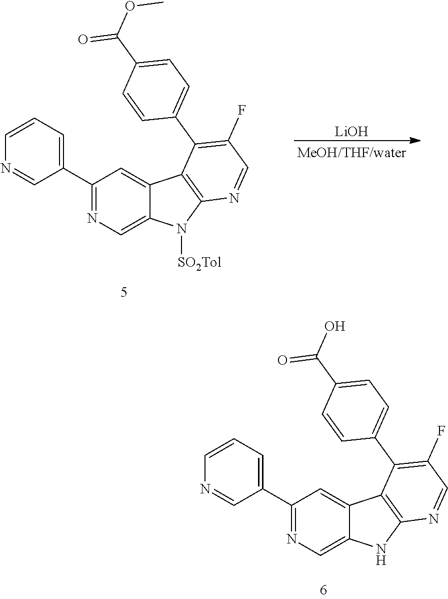

790 mg of methyl 4-{3-fluoro-9-[(4-methylphenyl)sulfonyl]-6-(pyridin-3-yl)-9H-pyrrolo-[2,3-b:5,4-c′]dipyridin-4-yl}benzoate in 10 ml of methanol and 20 ml of tetrahydrofuran and then 479 mg of lithium hydroxide dissolved in 15 ml of water are placed in a round-bottomed flask. The reaction mixture is stirred for 2 hours at room temperature, followed by addition of 30 ml of water and 10 ml of aqueous 2 M hydrochloric acid solution. The precipitate is filtered off by suction and dried under vacuum to give 340 mg of 4-[3-fluoro-6-(pyridin-3-yl)-9H-pyrrolo[2,3-b:5,4-c]dipyridin-4-yl]benzoic acid.

UPLC-MS-DAD-ELSD: Tr (min)=0.56; [M+H]⁺: m/z 385; [M−H]⁻ m/z 383; purity: 95%

1H NMR (400 MHz, DMSO-d6) ppm: 7.48 (dd, J=7.6, 4.9 Hz, 1H) 7.68 (s, 1H) 7.91 (d, J=8.1 Hz, 2H) 8.17 (d, J=8.1 Hz, 1H) 8.27 (d, J=8.3 Hz, 2H) 8.54 (d, J=4.4 Hz, 1H) 8.78 (d, J=2.2 Hz, 1H) 8.92 (s, 1H) 9.09 (s, 1H) 12.65 (s, 1H) 13.24 (br. s., 1H)

### Example 7

### 4-[3-fluoro-6-(pyridin-3-yl)-9H-pyrrolo[2,3-b:5,4-c′]dipyridin-4-yl]-N-(4-methylpiperazin-1-yl)benzamide

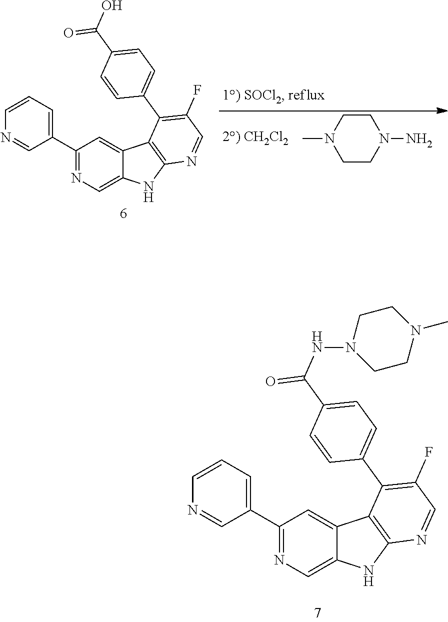

A mixture of 150 mg of 4-[3-fluoro-6-(pyridin-3-yl)-9H-pyrrolo[2,3-b:5,4-c′]dipyridin-4-yl]benzoic acid and 928 mg of thionyl chloride in a round-bottomed flask is refluxed for 12 hours and then evaporated under reduced pressure. 450 mg of 1-amino-4-methylpiperazine dissolved in 5 ml of dichloromethane are added to the residue. After stirring for 1 hour at room temperature, the reaction mixture is evaporated under reduced pressure, and the residue is taken up in dichloromethane and 150 g of silica are added. The deposit is concentrated under reduced pressure and is then purified by chromatography on a column of silica, eluting with a 98/2 to 90/10 dichloromethane/methanol mixture to give 34 mg of 4-[3-fluoro-6-(pyridin-3-yl)-9H-pyrrolo[2,3-b:5,4-c′]dipyridin-4-yl]-N-(4-methylpiperazin-1-yl)benzamide.

UPLC-MS-DAD-ELSD: Tr (min)=0.38; [M+H]⁺: m/z 482; [M−H]⁻ m/z 480; purity: 98%

1H NMR (400 MHz, DMSO-d6) ppm: 2.21 (s, 3H) 2.41-2.48 (m, 4H) 2.96 (t, J=4.6 Hz, 4H) 7.46 (dd, J=81, 4.6 Hz, 1H) 7.67 (s, 1H) 7.85 (d, J=8.3 Hz, 2H) 8.10 (d, J=8.3 Hz, 2H) 8.12-8.16 (m, 1H) 8.54 (dd, J=4.6, 1.2 Hz, 1H) 8.77 (d, J=2.4 Hz, 1H) 8.92 (d, J=2.0 Hz, 1H) 9.09 (s, 1H) 9.63 (s, 1H) 12.62 (br. s., 1H)

### Example 8

### 3-fluoro-4-iodo-6-(pyridin-3-yl)-9H-pyrrolo[2,3-b:5,4-c′]dipyridine

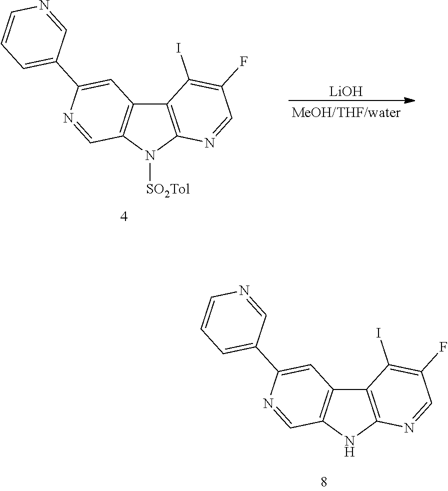

2.2 g of 3-fluoro-4-iodo-9-[(4-methylphenyl)sulfonyl]-6-(pyridin-3-yl)-9H-pyrrolo[2,3-b:5,4-c′]dipyridine in 50 ml of methanol and 100 ml of tetrahydrofuran and than 2.1 g of lithium hydroxide dissolved in 100 ml of water are placed in a round-bottomed flask. The reaction mixture is stirred for 3 hours at room temperature, followed by addition of 200 ml of water and aqueous 2 M hydrochloric acid solution to pH 6. The precipitate is filtered off by suction and dried under vacuum to give 1.16 g of 3-fluoro-4-iodo-6-(pyridin-3-yl)-9H-pyrrolo[2,3-b:5,4-c′]dipyridine.

UPLC-MS-DAD-ELSD: Tr (min)=2.91; [M+H]⁺: m/z 391; [M−H]⁻: m/z 389; purity: 98%

1H NMR (400 MHz, DMSO-d6): ppm 7.56 (dd, J=8.1, 4.6 Hz, 1H) 8.43 (dt, J=7.8, 2.0 Hz, 1H) 8.56 (s, 1H) 8.62 (dd, J=4.6, 1.2 Hz, 1H) 9.09 (s, 1H) 9.11 (s, 1H) 9.27 (d, J=2.0 Hz, 1H) 12.63 (br. s., 1H)

### Example 9

### N-[2-(dimethylamino)ethyl]-4-[3-fluoro-6-(pyridin-3-yl)-9H-pyrrolo-[2,3-b:5,4-c′]dipyridin-4-yl]benzamide

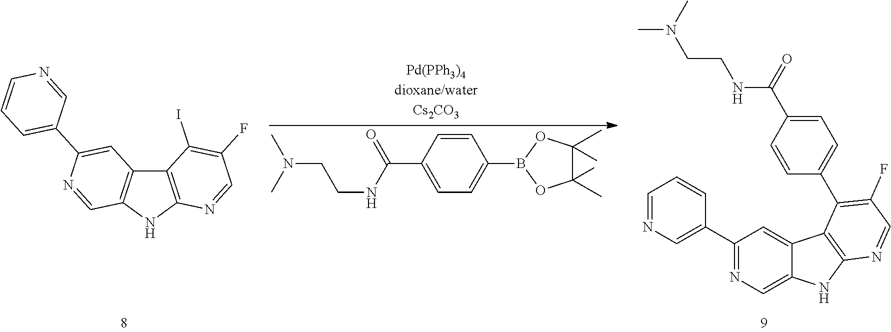

1.1 g of 3-fluoro-4-iodo-6-(pyridin-3-yl)-9H-pyrrolo[2,3-b:5,4-c′]dipyridine, 1.9 g N-(2-dimethylaminoethyl)-4-(4,4,5,5-tetramethyl-1,3,2-dioxaborolan-2-yl)benzamide, 326 mg of tetrakis(triphenylphosphine)palladium(0), 1.84 g of caesium carbonate in 15 ml of 1,4-dioxane and 2.5 ml of water are placed in a 20 ml reactor, and the tube is sealed and subjected to microwave irradiation for 1 hour at 130° C. The reaction mixture is filtered and then poured into 100 ml of water and 250 ml of ethyl acetate with vigorous stirring. After separation of the phases by settling, the aqueous phase is extracted with 100 ml of ethyl acetate and the combined organic phases are dried over magnesium sulfate, filtered and then evaporated under reduced pressure. The residue is purified by chromatography on a column of silica, eluting with a 100/0/0 to 90/10/0.2 dichloromethane/methanol/28% aqueous ammonia mixture; the product is suspended in 15 ml of ethyl acetate. After vigorous stirring for 16 hours at room temperature, the solid is filtered off by vacuum suction to give 1.02 g of N-[2-(dimethylamino)ethyl]-4-[3-fluoro-6-(pyridin-3-yl)-9H-pyrrolo[2,3-b:5,4-c′]dipyridin-4-yl]benzamide.

UPLC-MS-DAD-ELSD: Tr (min)=0.41; [M+H]⁺: 455 m/z; [M−H]⁻: 453 m/z; purity: 95%

1H NMR (400 MHz, DMSO-d6): ppm 2.22 (s, 6H) 2.45-2.49 (m, 2H) 3.44 (q, J=6.5 Hz, 2H) 7.46 (dd, J=7.8, 4.6 Hz, 1H) 7.68 (s, 1H) 7.87 (d, J=8.1 Hz, 2H) 8.11-8.15 (m, 1H) 8.16 (d, J=8.1 Hz, 2H) 8.54 (dd, J=4.6, 1.5 Hz, 1H) 8.61 (t, J=5.6 Hz, 1H) 8.77 (d, J=2.4 Hz, 1H) 8.91 (d, J=2.0 Hz, 1H) 9.08 (s, 1H) 12.61 (br. s., 1H)

### Example 10

### N-[3-(dimethylamino)propyl]-4-[3-fluoro-6-(pyridin-3-yl)-9H-pyrrolo[2,3-b:5,4-c′]dipyridin-4-yl]benzamide

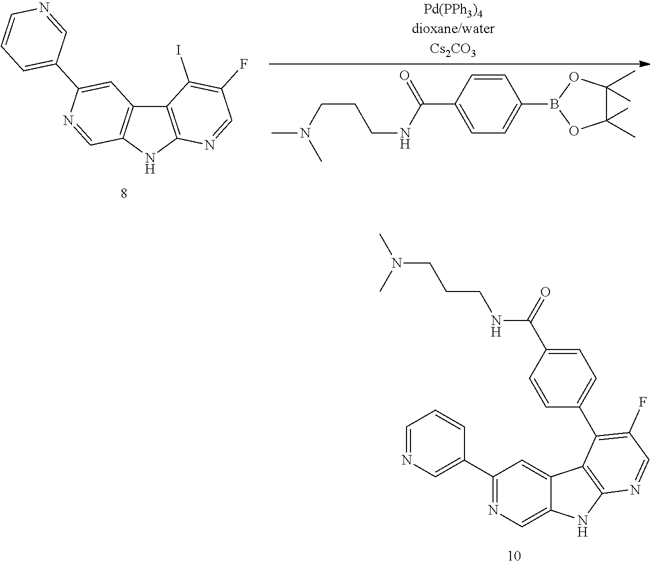

In a manner similar to that of Example 9, Example 10 is obtained starting with 150 mg of 3-fluoro-4-iodo-6-(pyridin-3-yl)-9H-pyrrolo[2,3-b:5,4-c′]dipyridine and 383 mg of N-(3-dimethylaminopropyl)-4-(4,4,5,5-tetramethyl-1,3,2-dioxaborolan-2-yl)benzamide.

UPLC-MS-DAD-ELSD: Tr (min)=2.21; [M+H]⁺: m/z 469; [M−H]⁻: m/z 467; purity: 98%

1H NMR (400 MHz, DMSO-d6): ppm 1.72 (quint., J=7.1 Hz, 2H) 2.16 (s, 6H) 2.31 (t, J=71 Hz, 2H) 3.35-3.41 (m, 2H) 7.46 (dd, J=7.9, 4.8 Hz, 1H) 7.68 (d, J=1.1 Hz, 1H) 7.87 (d, J=7.9 Hz, 2H) 8.12-815 (m, 1H) 8.16 (d, J=8.3 Hz, 2H) 8.53 (dd, J=4.6, 1.5 Hz, 1H) 8.73-8.77 (m, 1H) 8.77 (d, J=2.4 Hz, 1H) 8.91 (d, J=1.8 Hz, 1H) 9.09 (d, J=0.9 Hz, 1H) 12.62 (br. s., 1H)

### Examples 11 to 17

### General Procedure for Examples 11 to 17

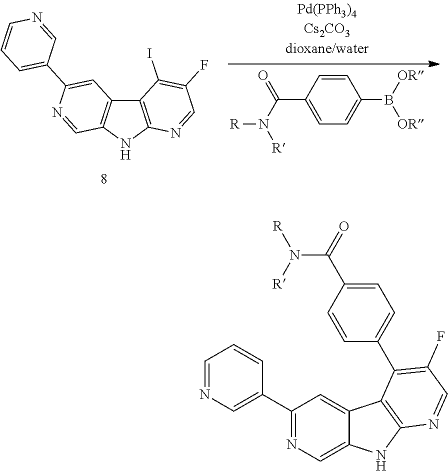

0.2 mmol of 3-fluoro-4-iodo-6-(pyridin-3-yl)-9H-pyrrolo[2,3-b:5,4-c′]dipyridine, 0.4 mmol of boronic reagent in 2 ml of 1,4-dioxane, 0.4 mmol of caesium carbonate in 0.5 ml of water, and 0.02 mmol of tetrakis(triphenylphosphine)palladium(0) in 0.5 ml of dimethylformamide are placed in a reactor under argon, and the tube is sealed and stirred at 110° C. for 18 hours. After cooling, the reaction mixture is diluted with 6 ml of 1,4-dioxane, 2 ml of methanol and 0.1 ml of trifluoroacetic acid and then treated with 150 mg of resin of propanethiol type grafted onto silica, for 4 hours at room temperature. The reaction mixture is filtered and then washed twice with a 4/1 1,4-dioxane/methanol mixture. After evaporating under reduced pressure, the residue is dissolved in 2 ml of dimethylformamide and 0.1 ml of trifluoroacetic acid, filtered off and then purified by preparative HPLC.

Amides 11 to 17 are detailed in Table 1.

TABLE 1

| Acid or | | | |
|---|---|---|---|
| boronic ester | Structure obtained | Name | Analysis |
| 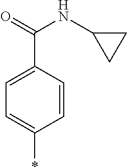 | 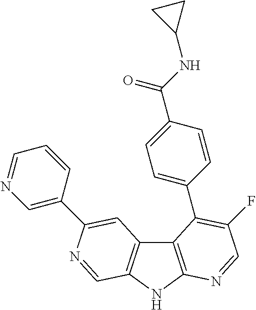 11 | N-cyclopropyl- 4-[3-fluoro-6- (pyridin-3-yl)- 9H-pyrrolo [2,3-b:5,4-c′] dipyridin-4-yl]- benzamide | UPLC-MS-DAD-ELSD: Tr (min) = 2.37; [M + H]^{+:} m/z 424.09; purity: 100% 1H NMR (400 MHz, DMSO- d6): δ ppm 0.62-0.69 (m, 2 H) 0.72-0.81 (m, 2 H) 2.91- 3.02 (m, 1 H) 7.48 (dd, J = 9.2, 4.6 Hz, 1 H) 7.69 (s, 1 H) 7.87 (d, J = 8.2 Hz, 2 H) 8.16 (d, J = 8.7 Hz, 2 H) 8.56 (d, J = 6.1 Hz, 1 H) 8.67 (d, J = 4.8 Hz, 1 H) 8.79 (d, J = 2.5 Hz, 1 H) 8.93 (d, J = 2.3 Hz, 1 H) 9.11 (s, 1 H) 12.63 (s, 1H) |
| 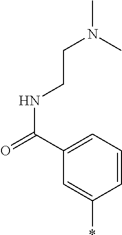 | 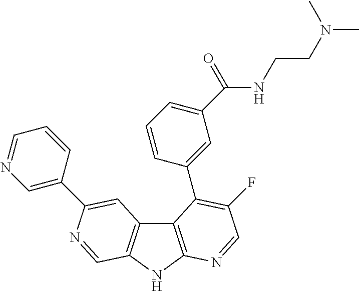 12 | N-[2-(dimethyl- amino)ethyl]-3- [3-fluoro-6- (pyridin-3-yl)- 9H-pyrrolo [2,3-b:5,4-c′] dipyridin-4-yl]- benzamide | UPLC-MS-DAD-ELSD: Tr (min) = 2.10; [M + H]^{+:} m/z 455.11; purity: 100% 1H NMR (400 MHz, DMSO- d6): δ ppm 2.84 (s, 6 H) 3.30 (t, J = 5.2 Hz, 2H) 3.61-3.71 (m, 2 H) 7.58 (dd, J = 8.3, 5.0 Hz, 1 H) 7.78 (s, 1 H) 7.91 (t, J = 8.0 Hz, 1 H) 8.03 (d, J = 7.4 Hz, 1 H) 8.23 (d, J = 8.1 Hz, 1 H) 8.31 (d, J = 8.1 Hz, 1 H) 8.33 (s, 1 H) 8.63 (d, J = 3.8 Hz, 1 H) 8.86 (d, J = 2.3 Hz, 1 H) 8.96 (t, J = 6.0 Hz, 1 H) 9.03 (br. s., 1 H) 9.17 (s, 1 H) 9.40 (br. s., 1 H) 12.75 (s, 1 H) |
| 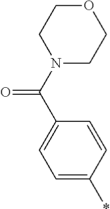 | 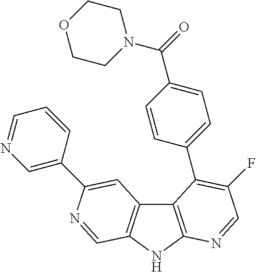 13 | {4-[3-fluoro-6- (pyridin-3-yl)- 9H-pyrrolo [2,3-b:5,4-c′] dipyridin-4- yl]-phenyl} (morpholin-4-yl) methanone | UPLC-MS-DAD-ELSD: Tr (min) = 2.55; [M + H]^{+:} m/z 454.07; purity: 100% 1H NMR (400 MHz, DMSO- d6): δ ppm 3.63-3.78 (masked m, 8 H) 7.62 (dd, J = 8.3, 5.2 Hz, 1 H) 7.74 (s, 1 H) 7.78 (d, J = 8.7 Hz, 2 H) 7.83- 7.93 (m, J = 8.2 Hz, 2 H) 8.35 (d, J = 8.4 Hz, 1 H) 8.65 (d, J = 6.1 Hz, 1 H) 8.81 (d, J = 2.3 Hz, 1 H) 8.99 (s, 1 H) 9.13 (s, 1 H) 12.69 (s, 1 H) |
| 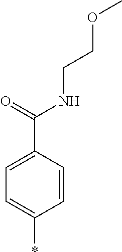 | 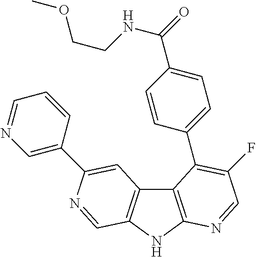 14 | 4-[3-fluoro-6- (pyridin-3-yl)- 9H-pyrrolo [2,3-b:5,4-c′] dipyridin-4-yl]- N-(2-methoxy- ethyl)benzamide | UPLC-MS-DAD-ELSD: Tr (min) = 2.34; [M + H]^{+:} m/z 442.05; purity: 100% 1H NMR (400 MHz, DMSO- d6): δ ppm 3.30 (s, 3 H) 3.46- 3.56 (m, 4 H) 7.73 (dd, J = 8.0, 5.4 Hz, 1 H) 7.81 (s, 1 H) 7.93 (d, J = 8.4 Hz, 2 H) 8.24 (d, J = 8.6 Hz, 2 H) 8.43 (d, J = 8.6 Hz, 1 H) 8.72 (d, J = 4.3 Hz, 1H) 8.82 (t, J = 5.1 Hz, 1 H) 8.86 (d, J = 2.5 Hz, 1 H) 9.11 (br. s., 1 H) 9.19 (s, 1 H) 12.77 (s, 1 H) |
| 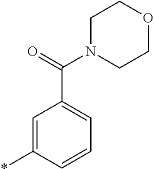 | 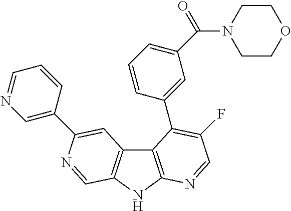 15 | {3-[3-fluoro-6- (pyridin-3-yl)- 9H-pyrrolo [2,3-b:5,4-c′] dipyridin-4- yl]phenyl}- (morpholin-4-yl) methanone | UPLC-MS-DAD-ELSD: Tr (min) = 2.35; [M + H]^{+:} m/z 454.07; purity: 94% 1H NMR (400 MHz, DMSO- d6) : δ ppm 3.54-3.89 (masked m, 8 H) 7.70 (66, J = 8.2, 4.8 Hz, 1 H) 7.73- 7.79 (m, 1 H) 7.80-7.94 (m, 4H) 8.54 (d, J = 7.9 Hz, 1 H) 8.68 (br. s., 1 H) 8.83 (d, J = 2.5 Hz, 1 H) 9.15 (s, 1 H) 9.20 (br. s., 1 H) 12.73 (s, 1 H) |
| 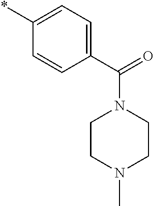 | 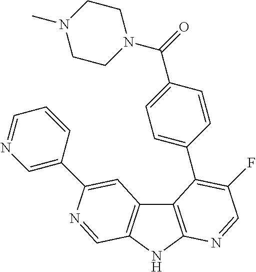 16 | {4-[3-fluoro-6- (pyridin-3-yl)- 9H-pyrrolo [2,3-b:5,4-c′] dipyridin-4-yl] phenyl}- (4-methyl- piperazin-1-yl) methanone | UPLC-MS-DAD-ELSD: Tr (min) = 2.05; [M + H]^{+:} m/z 467.12; purity: 100% 1H NMR (400 MHz, DMSO- d6): δ ppm 2.88 (s, 3 H) 3.36- 3.80 (masked m, 8 H) 7.55 (dd, J = 8.5, 5.0 Hz, 1 H) 7.77 (s, 1 H) 7.85 (d, J = 8.4 Hz, 2 H) 7.90-8.01 (m, 2 H) 8.30 (d, J = 8.7 Hz, 1 H) 8.63 (d, J = 3.8 Hz, 1 H) 8.85 (d, J = 2.3 Hz, 1 H) 9.01 (br. s., 1 H) 9.16 (s, 1 H) 12.71 (s, 1 H) |
| 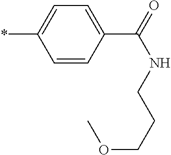 | 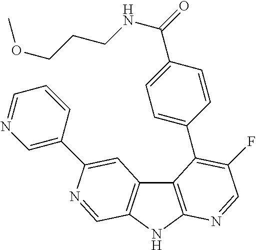 17 | 4-[3-fluoro-6- (pyridin-3-yl)- 9H-pyrrolo [2,3-b:5,4-c′] dipyridin- 4-yl]-N-(3- methoxypropyl) benzamide | UPLC-MS-DAD-ELSD: Tr (min) = 2.39; [M + H]^{+:} m/z 456.09; purity 93% 1H NMR (400 MHz, DMSO- d6): δ ppm 1.81 (quint., J = 6.9 Hz, 2 H) 3.27 (s, 3 H) 3.35- 3.46 (m, 4 H) 7.47 (dd, J = 8.3, 5.0 Hz, 1 H) 7.69 (s, 1 H) 7.88 (d, J = 8.4 Hz, 2 H) 8.12- 8.22 (m, 3 H) 8.56 (dd, J = 4.8, 1.5 Hz, 1 H) 8.71 (t, J = 5.9 Hz, 1 H) 8.80 (d, J = 2.5 Hz, 1 H) 8.94 (d, J = 2.3 Hz, 1 H) 9.12 (s, 1 H) 12.64 (s, 1 H) |

### Example 18

### 4-[3-fluoro-6-(pyridin-3-yl)-9H-pyrrolo[2,3-b:5,4-c′]dipyridin-4-yl]-benzoyl chloride

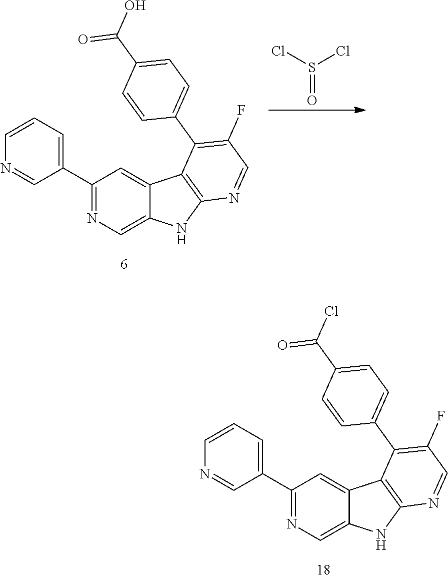

A mixture of 100 mg of 4-[3-fluoro-6-(pyridin-3-yl)-9H-pyrrolo[2,3-b:5,4-c′]dipyridin-4-yl]benzoic acid and 12 ml of thionyl chloride is heated at 70° C. for 20 hours. The reaction mixture is concentrated to dryness under reduced pressure to give 105 mg of 4-[3-fluoro-0′(pyridin-3-yl)-9H-pyrrolo[2,3-b:5,4-c′]dipyridin-4-yl]benzoyl chloride in the form of a yellow powder. The product is characterized by addition of methanol to give the corresponding ester.

### Examples 19 to 28

### General Procedure for Examples 19 to 28

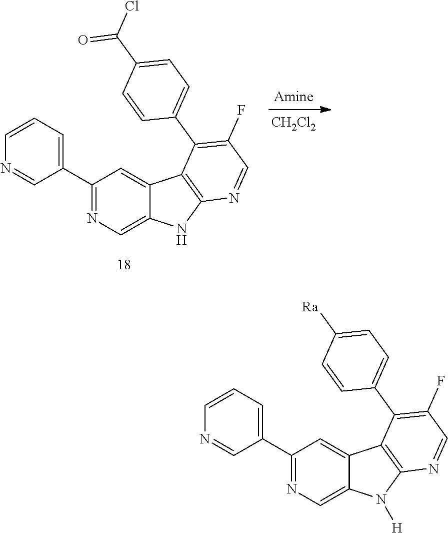

A mixture of 105 mg of 4-[3-fluoro-6-(pyridin-3-yl)-9H-pyrrolo[2,3-b:5,4-c′]dipyridin-4-yl]benzoyl chloride, 10 equivalents of amine (cf. Table 2) and 10 ml of dichloromethane is stirred at room temperature for 20 hours and the reaction mixture is then concentrated to dryness under reduced pressure. The residue is taken up in 300 ml of water and 300 ml of ethyl acetate. After separation of the phases by settling, the aqueous phase is extracted with 100 ml of ethyl acetate and the organic phases are then combined and concentrated under reduced pressure.

The amides are detailed in Table 2 (yield between 10% and 79% depending on the reagent).

TABLE 2

| | Structure obtained and | | |
|---|---|---|---|
| Reagent | example No. | Name | Analysis |
| 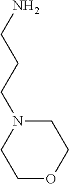 | 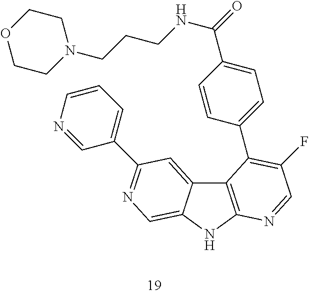 | 4-[3-fluoro-6- (pyridin-3-yl)-9H- pyrrolo[2,3-b:5,4- c′]dipyridin-4-yl]-N- [3-(morpholin-4- yl)propyl] benzamide | UPLC-MS-DAD-ELSD: Tr (min) = 0.42; [M + H]^{+:} m/z 511; [M − H]^{-:} m/z 509; purity: 95% 1H NMR (400 MHz, DMSO- d6): δ ppm 1.76 (quint, J = 7.2 Hz, 2 H) 2.35-2.41 (m, 6 H) 3.38 (q, J = 6.6 Hz, 2 H) 3.57 (t, J = 4.7 Hz, 4 H) 7.46 (dd, J = 7.8, 4.9 Hz, 1 H) 7.67 (s, 1 H) 7.87 (d, J = 7.8 Hz, 2 H) 8.12-8.15 (m, 1 H) 8.16 (d, J = 8.3 Hz, 2 H) 8.53 (dd, J = 4.8, 1.6 Hz, 1 H) 8.71 (t, J = 5.6 Hz, 1 H) 8.77 (d, J = 2.2 Hz, 1 H) 8.90 (d, J = 2.0 Hz, 1 H) 9.08 (s, 1 H) 11.81 (br. s., 1 H) |

| | | | |
|---|---|---|---|
| 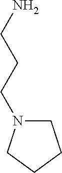 | 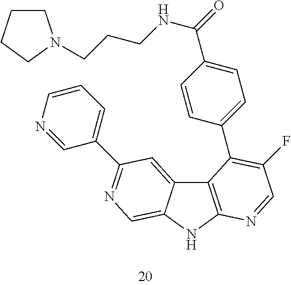 | 4-[3-fluoro-6- (pyridin-3-yl)-9H- pyrrolo[2,3-b:5,4- c′]dipyridin-4-yl]-N- [3-(pyrrolidin-1- yl)propyl] benzamide | UPLC-MS-DAD-ELSD: Tr (min) = 0.44; [M + H]^{+:} m/z 595; purity: 70% 1H NMR (400 MHz, DMSO- d6): δ ppm 1.64-1.70 (m, 4 H) 1.76 (quint., J = 7.0 Hz, 2 H) 2.42-2.47 (m, 4 H) 2.48- 2.54 (m, 2 H) 3.35-3.43 (m, 2 H) 7.46 (dd, J = 7.9, 4.8 Hz, 1 H) 7.67 (d, J = 1.0 Hz, 1 H) 7.86 (d, J = 7.8 Hz, 2 H) 8.10- 8.18 (m, 3 H) 8.53 (dd, J = 4.6, 1.7 Hz, 1 H) 8.77 (d, J = 2.2 Hz, 1 H) 8.77-8.82 (m, 1 H) 8.91 (d, J = 2.2 Hz, 1 H) 9.09 (d, J = 1.0 Hz, 1 H) 12.42 (br. s., 1 H) |
| 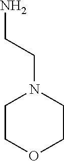 | 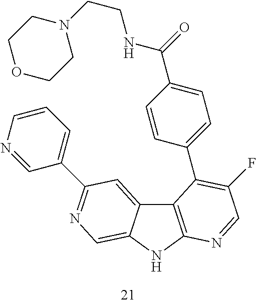 | 4-[3-fluoro-6- (pyridin-3-yl)-9H- pyrrolo[2,3-b:5,4- c′]dipyridin-4-yl]-N- [2-(morpholin-4- yl)ethyl] benzamide | UPLC-MS-DAD-ELSD: Tr (min) = 2.23; [M + H]^{+:} m/z 497; [M − H]^{-:} m/z 495; purity: 95% 1H NMR (400 MHz, DMSO- d6): δ ppm 2.44-2.57 (m, 8 H) 3.48 (q, J = 6.4 Hz, 2 H) 3.60 (t, J = 4.4 Hz, 2H) 7.46 (dd, J = 7.8, 4.9 Hz, 1 H) 7.68 (s, 1 H) 7.87 (d, J = 8.3 Hz, 2 H) 8.12-8.14 (m, 1 H) 8.16 (d, J = 7.8Hz, 2 H) 8.53 (dd, J = 4.9, 1.5 Hz, 1 H) 8.63 (t, J = 5.6 Hz, 1 H) 8.77 (d, J = 2.0 Hz, 1 H) 8.91 (d, J = 2.0 Hz, 1 H) 9.09 (s, 1 H) |
| 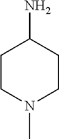 | 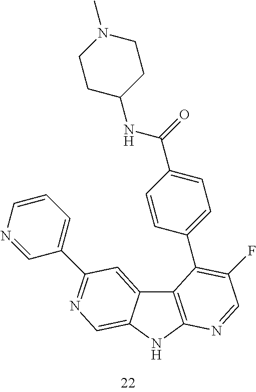 | 4-[3-fluoro-6- (pyridin-3-yl)-9H- pyrrolo[2,3-b:5,4- c′]dipyridin-4-yl]-N- (1-methylpiperidin- 4-yl)benzamide | UPLC-MS-DAD-ELSD: Tr (min) = 0.42; [M + H]^{+:} m/z 481; [M − H]^{-:} m/z 479; purity: 80% 1H NMR (400 MHz, DMSO- d6): δ ppm 1.55-1.72 (m, 2 H) 1.77-1.89 (m, 2 H) 1.92- 2.06 (m, 2 H) 2.19 (s, 3 H) 2.75-2.88 (m, 2 H) 3.74-3.91 (m, 1 H) 7.40-7.53 (m, 1 H) 7.68 (s, 1 H) 7.86 (d, J = 7.6 Hz, 2 H) 8.07-8.15 (m, 1 H) 8.17 (d, J = 8.3 Hz, 2 H) 8.43-8.49 (m, 1 H) 8.50-8.59 (m, 1 H) 8.77 (s, 1 |
| 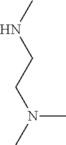 | 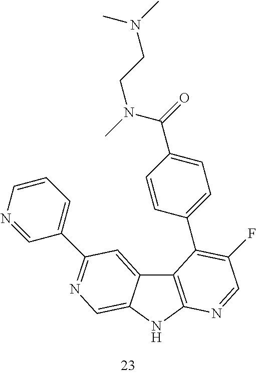 | N-[2-(dimethyl- amino)ethyl]-4-[3- fluoro-6-(pyridin-3- yl)-9H-pyrrolo[2,3- b:5,4-c′]dipyridin-4- yl]-N- methylbenzamide | UPLC-MS-DAD-ELSD: Tr (min) = 0.41; [M + H]^{+:} m/z 469; [M − H]^{-:} m/z 467; purity: 95% 1H NMR (400 MHz, DMSO- d6+ ACOD): δ ppm 2.89 (br. s., 6 H) 3.12 (s, 3 H) 3.38 (br. s., 2 H) 3.90 (br. s., 2 H) 7.46 (dd, J = 7.7, 4.8 Hz, 1 H) 7.69 (br. s., 1 H) 7.82 (d, J = 8.1 Hz, 2 H) 7.87 (d, J = 7.8 Hz, 2 H) 8.19 (d, J = 7.6 Hz, 1 H) 8.55 (dd, J = 4.6, 1.2 Hz, 1 H) 8.76 (d, J = 2.4 Hz, 1 H) 8.92 (br. s., 1 H) 9.10 (d, J = 1.0 Hz, 1 H) |
| 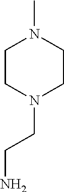 | 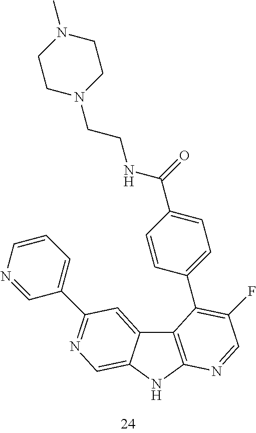 | 4-[3-fluoro-6- (pyridin-3-yl)-9H- pyrrolo[2,3-b:5,4- c′]dipyridin-4-yl]-N- [2-(4-methyl- piperazin-1-yl)- ethyl]benzamide | UPLC-MS-DAD-ELSD: Tr (min) = 0.41; [M + H]^{+:} m/z 510; [M − H]^{-:} m/z 508; purity: 90% 1H NMR (400 MHz, DMSO- d6): δ ppm 2.15 (s, 3 H) 2.25- 2.38 (m, 4 H) 2.45-2.58 (m, 6 H) 3.41-3.51 (m, 2 H) 7.46 (dd, J = 7.8, 4.6 Hz, 1 H) 7.68 (d, J = 1.0 Hz, 1 H) 7.87 (d, J = 7.8 Hz, 2 H) 8.10-8.19 (m, 3H) 8.54 (dd, J = 4.8, 1.6 Hz, 1 H) 8.61 (t, J = 5.1 Hz, 1 H) 8.77 (d, J = 2.2 Hz, 1 H) 8.91 (dd, J = 2.2, 0.7 Hz, 1H) 9.09 (d, J = 1.0 Hz, 1 H) 12.61 (br. s., 1 H) |
| 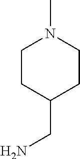 | 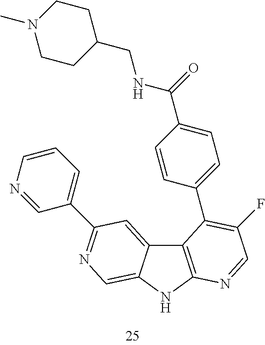 | 4-[3-fluoro-6- (pyridin-3-yl)-9H- pyrrolo[2,3-b:5,4- c′]dipyridin-4-yl]-N- [(1-methylpiperidin- 4-yl)methyl] benzamide | UPLC-MS-DAD-ELSD: Tr (min) = 2.23; [M + H]^{+:} m/z 495; [M − H]^{-:} m/z 493; purity: 97% 1H NMR (400 MHz, DMSO- d6): δ ppm 1.24 (qd, J = 12.1, 3.2 Hz, 2 H) 1.52-1.64 (m, 1 H) 1.65-1.76 (m, 2 H) 1.80- 1.94 (m, 2 H) 2.17 (s, 3 H) 2.74-2.85 (m, 2 H) 3.24 (t, J = 6.2 Hz, 2 H) 7.45 {dd, J = 8.1.4.6 Hz, 1 H) 7.65 (s, 1 H) 7.86 (d, J = 8.1 Hz, 2 H) 8.13 (dt, J = 8.1, 1.8 Hz, 1 H) 8.17 (d, J = 8.3 Hz, 2 H) 8.54 (dd, J = 4.9, 1.5 Hz, 1 H) 8.68 (t, J = 5.5 Hz, 1 H) 8.77 (d, J = 2.4 Hz, 1 H) 8.91 (d, J = 2.2 Hz, 1 H) 9.08 (s, 1 H) 12.62 (br. s., 1 H) |
| 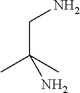 | 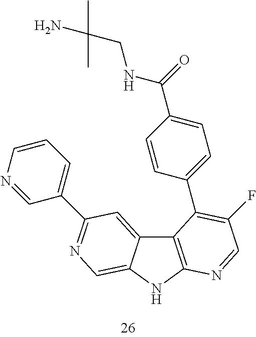 | N-(2-amino-2- methylpropyl)-4-[3- fluoro-6-(pyridin-3- yl)-9H-pyrrolo[2,3- b:5,4-c′]dipyridin-4- yl]benzamide | UPLC-MS-DAD-ELSD: Tr (min) = 2.21; [M + H]^{+:} m/z 455; [M − H]^{-:} m/z 453; purity: 97% 1H NMR (400 MHz, DMSO- d6): δ ppm 1.08 (s, 6 H) 3.25- 3.28 (m, 2 H) 7.44 (dd, J = 8.1, 4.6 Hz, 1 H) 7.68 (s, 1 H) 7.87 (d, J = 7.8 Hz, 2 H) 8.13 (dt, J = 8.0, 2.0 Hz, 1 H) 8.19 (d, J = 8.3 Hz, 2 H) 8.48 (t, J = 5.3 Hz, 1 H) 8.54 (dd, J = 4.8, 1.6 Hz, 1 H) 8.77 (d, J = 2.2 Hz, 1 H) 8.93 (d, J = 2.0 Hz, 1 H) 9.09 (s, 1 H) |
| 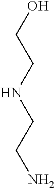 | 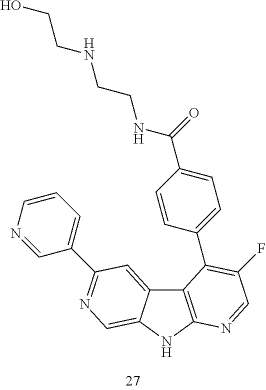 | 4-[3-fluoro-6- (pyridin-3-yl)-9H- pyrrolo[2,3-b:5,4- c′]dipyridin-4-yl]-N- {2-[(2-hydroxy- ethyl)amino]ethyl}- benzamide | UPLC-MS-DAD-ELSD: Tr (min) = 2.12; [M + H]^{+:} m/z 471; [M − H]⁻: m/z 469; purity: 95% 1H NMR (400 MHz, DMSO- d6): δ ppm 2.62-2.69 (m, 2 H) 2.76 (t, J = 6.4 Hz, 2 H) 3.39-3.51 (m, 4 H) 4.46 (br. s., 1 H) 7.47 (dd, J = 8.1, 4.9 Hz, 1 H) 7.69 (d, J = 1.0 Hz, 1 H) 7.87 (d, J = 7.6 Hz, 2 H) 8.12-8.15 (m, 1 H) 8.17 (d, J = 8.6 Hz, 2 H) 8.54 (dd, J = 4.8, 1.6 Hz, 1 H) 8.64 (t, J = 5.9 Hz, 1 H) 8.77 (d, J = 2.2 Hz, 1 H) 8.92 (d, J = 1.5 Hz, 1 H) 9.09 (d, J = 1.0 Hz, 1 H) |
| 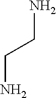 | 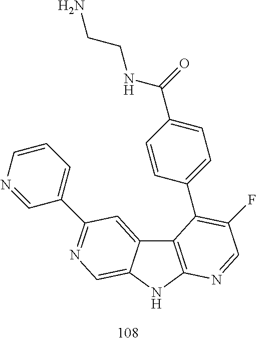 | N-(2-aminoethyl)-4- [3-fluoro-6-(pyridin- 3-yl)-9H-pyrrolo- [2,3-b:5,4-c′]di- pyridin-4-yl] benzamide | UPLC-MS-DAD-ELSD: Tr (min) = 0.39; [M + H]^{+:} m/z 427; [M − H]^{-:} m/z 425; purity: 95% 1H NMR (400 MHz, DMSO- d6): δ ppm 2.75 (t, J = 6.6 Hz, 2 H) 3.33-3.38 (m, 2 H) 7.46 (dd, J = 8.2, 4.8 Hz, 1 H) 7.69 (s, 1 H) 7.86 (d, J = 7.8 Hz, 2 H) 8.14 (dt, J = 8.0, 2.0 Hz, 1 H) 8.18 (d, J = 8.6 Hz, 2 H) 8.53 (d, J = 4.2 Hz, 1 H) 8.62 (t, J = 5.7 Hz, 1 H) 8.74-8.77 (m, 1 H) 8.92 (d, J = 1.7 Hz, 1 H) 9.08 (s, 1 H) |

### 5-chloro-2-methoxy-4-(trimethylstannyl)pyridine A2

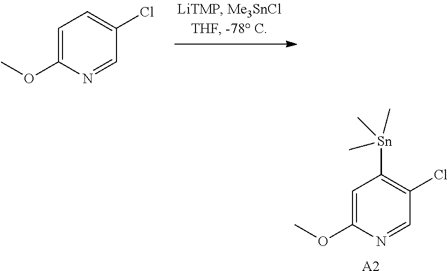

A mixture of 10 g of 5-chloro-2-methoxypyridine and 220 ml of tetrahydrofuran is cooled to −78° C., followed by gradual addition of a solution freshly prepared from 14.1 ml of 2,2,6,6-tetramethylpiperidine in 50 ml of tetrahydrofuran and 36.4 ml of 2.3 N n-butyllithium in hexane. After stirring for 4 hours at −78° C., 17.3 g of trimethyltin chloride dissolved in 30 ml of tetrahydrofuran are added to the reaction mixture. The reaction mixture is stirred at room temperature for 18 hours and then treated with 200 ml of water and 200 ml of aqueous 10% ammonium chloride solution and extracted with 500 ml and then 200 ml of ethyl acetate. The combined organic phases are dried over magnesium sulfate, filtered and then concentrated to dryness under reduced pressure. The residue is purified by chromatography on a column of silica, eluting with dichloromethane to give 17.7 g of 5-chloro-2-methoxy-4-(trimethylstannyl)pyridine A2 in the form of a colourless oil.

UPLC-MS-DAD-ELSD: Tr (min)=1.24; [M+H]⁺: m/z 308; purity: 98%

1H NMR (400 MHz, DMSO-d6): ppm 0.16 (t, J=29.6 Hz, 9H) 3.62 (s, 3H) 6.61 (t, J=20.5 Hz, 1H) 7.90 (t, J=8.3 Hz, 1H)

### Example 29

### 5′-chloro-5-fluoro-2′-methoxy-3,4′-bipyridin-2-amine

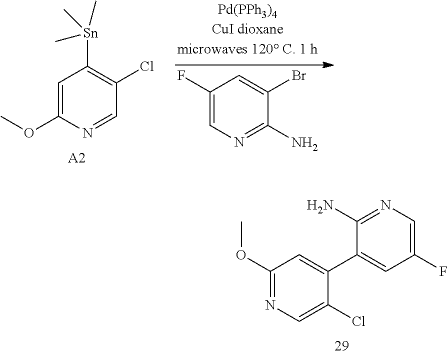

A mixture of 13.2 g of 5-chloro-2-methoxy-4-(trimethylstannyl)pyridine, 7.5 g of 2-amino-3-bromo-5-fluoropyridine, 3.1 g of tetrakis(triphenylphosphine)palladium(0) and 1.6 g of copper iodide in 100 ml of 1,4-dioxane is refluxed for 18 hours. The reaction mixture is hydrolysed with a mixture of 200 ml of aqueous 10% sodium hydrogen carbonate solution and 100 ml of water and is than extracted with twice 200 ml of ethyl acetate. The combined organic phases are dried over magnesium sulfate, filtered and then concentrated to dryness under reduced pressure. The residue is purified by trituration in 120 ml of ethyl acetate to give 8.7 g of 5′-chloro-5-fluoro-2′-methoxy-3,4′-bipyridin-2-amine in the form of a colourless oil.

UPLC-MS-DAD-ELSD: Tr (min)=0.94; [M+H]⁺: m/z 253; purity: 72%

1H NMR (400 MHz, DMSO-d6): ppm 3.89 (s, 3H) 5.65 (s, 2H) 6.86 (s, 1H) 7.33 (dd. J=8.7, 3.1 Hz, 1H) 8.01 (d, J=2.9 Hz, 1H) 8.32 (s, 1H)

### Example 30

### 3-fluoro-6-methoxy-9H-pyrrolo[2,3-b:5,4-c′]dipyridine

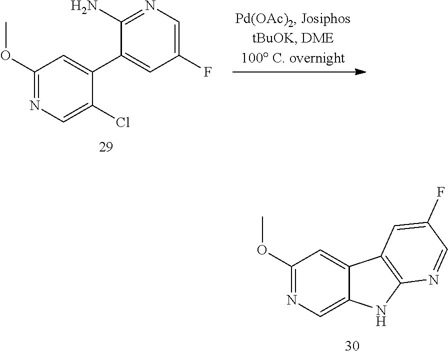

1.12 g of (R)-(−)-1-[(S)-2-(dicyclohexylphosphino)ferrocenyl]ethyldi-tert-butylphosphine and 0.40 g of palladium (II) acetate in 15 ml of anhydrous 1,2-dimethoxyethane are placed in a dry tube under an argon atmosphere and stirred for 10 minutes at 40° C.

6.38 g of 5′-chloro-5-fluoro-2′-methoxy-3,4′-bipyridin-2-amine in 80 ml of anhydrous 1,2-dimethoxyethane are placed in a 250 ml reactor under argon, and the solution prepared previously is added, followed by addition of 5.64 g of potassium tert-butoxide. The reaction mixture is refluxed for 18 hours, followed by addition of a further 3 ml of freshly prepared catalyst solution of the same concentration and 1.41 g of potassium tert-butoxide. After refluxing for 6 hours, 10 ml of methanol and 300 ml of ethyl acetate are added to the reaction mixture. The organic phase is washed with 200 ml of aqueous 5% sodium hydrogen carbonate solution and then dried over magnesium sulfate, filtered and concentrated to dryness under reduced pressure. The residue is purified by trituration in 100 ml of ethyl acetate to give 2.23 g of 3-fluoro-6-methoxy-9H-pyrrolo[2,3-b:5,4-c′]dipyridine.

UPLC-MS-DAD-ELSD: Tr (min)=0.58; [M+H]⁺: m/z 218; purity: 98%

1H NMR (400 MHz, DMSO-d6): ppm 3.91 (s, 3H) 7.58 (s, 1H) 8.49 (s, 1H) 8.54-8.61 (m, 2H) 11.85 (br, s., 1H)

### Example 31

### 3-fluoro-9H-pyrrolo[2,3-b:5,4-c′]dipyridin-6-ol

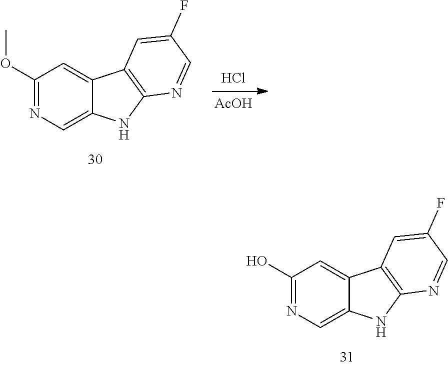

600 mg of 3-fluoro-6-methoxy-9H-pyrrolo[2,3-b:5,4-c′]dipyridine in 3 ml of acetic acid and 2 ml of aqueous 37% hydrochloric acid solution are placed in a reactor, and the tube is sealed and subjected to microwave irradiation for 30 minutes at 130° C. The reaction mixture is filtered by vacuum suction and then washed with ethyl ether to give 755 mg of 3-fluoro-9H-pyrrolo[2,3-b:5,4-c′]dipyridin-6-ol in the form of a yellow solid.

UPLC-MS-DAD-ELSD (LS): Tr (min)=0.45; [M+H]⁺: m/z 204; purity: 91%

1H NMR (400 MHz, DMSO-d6): ppm 5.47 (br, s., 1H) 7.92 (s, 1H) 8.52 (s, 1H) 8.75 (dd, J=2.7, 1.7 Hz, 1H) 8.87 (dd, J=8.7, 2.8 Hz, 1H) 12.52 (s, 1H)

### Example 32

### 3-fluoro-9H-pyrrolo[2,3-b:5,4-c′]dipyridin-6-yl trifluoromethanesulfonate

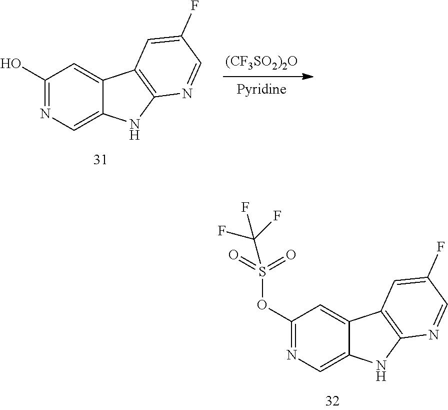

A mixture of 755 mg of 3-fluoro-9H-pyrrolo[2,3-b:5,4-c′]dipyridin-6-ol in 15 ml of pyridine and 4.32 ml of anhydrous trifluoromethanesulfonic acid is stirred at room temperature for 30 minutes. The reaction mixture is diluted with 100 ml of ethyl acetate and washed with 100 ml of saturated aqueous sodium bicarbonate solution. After separation of the phases by settling, the aqueous phase is extracted with 100 ml of ethyl acetate and the organic phases are then combined, dried over magnesium sulfate, filtered and then concentrated under vacuum to give 978 mg of 3-fluoro-9H-pyrrolo[2,3-b:5,4-c′]dipyridin-6-yl trifluoromethanesulfonate in the form of a brown solid.

UPLC-MS-DAD-ELSD: Tr (min)=4.22; [M+H]⁺: m/z 336; [M−H]⁻: m/z 334; purity: 98%

1H NMR (400 MHz, DMSO-d) ppm 8.41 (d, J=0.7 Hz, 1H) 8.71-8.78 (m, 3H) 12.69 (br. s., 1H)

### Example 33

### 3-fluoro-6-(1-methyl-1H-pyrazol-4-yl)-9H-pyrrolo[2,3-b:5,4-c]dipyridine

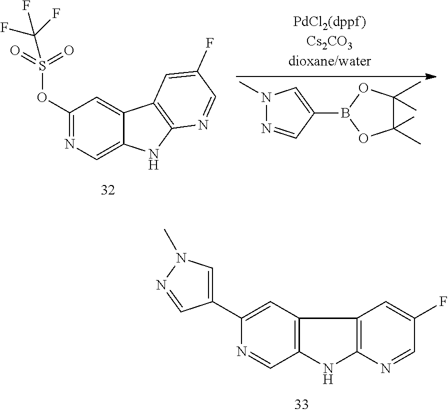

960 mg of 3-fluoro-9H-pyrrolo[2,3-b:5,4-c′]dipyridin-6-yl trifluoromethanesulfonate, 43.7 mg of 1,1′-bis(diphenylphosphino)ferrocenedichloropalladium(II), 372 mg of 1-methyl-4-(4,4,5,5-tetramethyl-1,3,2-dioxaborolan-2-yl)-1H-pyrazole, and 1.17 g of caesium carbonate in 10 ml of 1,4-dioxane and 2.5 ml of water are placed in a reactor, and the tube is sealed and subjected to microwave irradiation at 125° C. for 1 hour. The reaction mixture is diluted with 50 ml of ethyl acetate and washed with 50 ml of water. After separation of the phases by settling, the aqueous phase is extracted with 100 ml of ethyl acetate and the organic phases are then combined, dried over magnesium sulfate, filtered and then concentrated under vacuum. The residue is purified by chromatography on a column of silica, eluting with ethyl acetate to give 168 mg of 3-fluoro-6-(1-methyl-1H-pyrazol-4-yl)-9H-pyrrolo[2,3-b:5,4-c′]dipyridine in the form of a yellow solid.

UPLC-MS-DAD-ELSD: Ti (min)=2.24; [M+H]⁺: m/z 268; [M−H]⁻: m/z 266; purity: 98%

1H NMR (400 MHz, DMSO-d6): ppm 3.91 (s, 3H) 7.96 (d, J=0.5 Hz, 1H) 8.19 (s, 1H) 8.41 (d, J=1.0 Hz, 1H) 8.55 (dd, J=8.8, 2.9 Hz, 1H) 8.59-8.61 (m, 1H) 8.86 (d, J=1.2 Hz, 1H) 12.14 (br. S., 1H)

### Example 34

### 3-fluoro-9-[(4-methylphenyl)sulfonyl]-6-(1-methyl-1H-pyrazol-4-yl)-9H-pyrrolo[2,3-b:5,4-c′]dipyridine

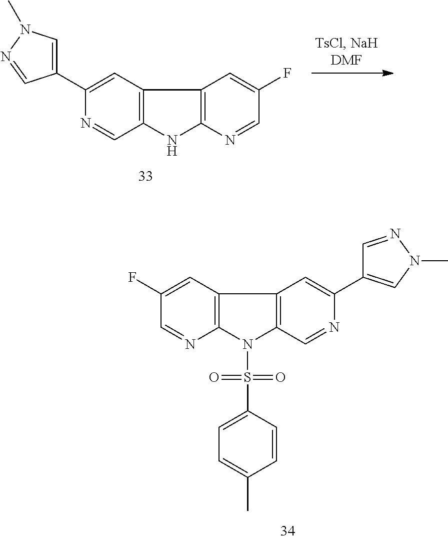

168 mg of 3-fluoro-6-(1-methyl-1H-pyrazol-4-yl)-9H-pyrrolo[2,3-b:5,4-c′]dipyridine in 10 ml of dimethylformamide are placed in a 100 ml round-bottomed flask under argon and 11 mg of sodium hydride are added. The reaction mixture is stirred for 3 hours at room temperature, followed by addition of 240 mg of para-toluenesulfonyl chloride. The reaction mixture is stirred for 45 minutes at room temperature and is then poured into a mixture of water and sodium hydrogen carbonate solution: a white precipitate appears. After stirring, it is filtered off by vacuum suction and then washed with diethyl ether. The white solid obtained is purified by chromatography on a column of silica, eluting with ethyl acetate, to give 225 mg of 3-fluoro-9-[(4-methylphenyl)sulfonyl]-6-(1-methyl-1H-pyrazol-4-yl)-9H-pyrrolo[2,3-b:5,4-c′]dipyridine in the form of a white solid.

UPLC-MS-DAD-ELSD (LS): Tr (min)=1.26; [M+H]⁺: m/z 422.26; purity: 93%

### Example 35

### 3-fluoro-4-iodo-9-[(4-methylphenyl)sulfonyl]-6-(1-methyl-1H-pyrazol-4-yl)-9H-1-pyrrolo[2,3-b:5,4-c′]dipyridine

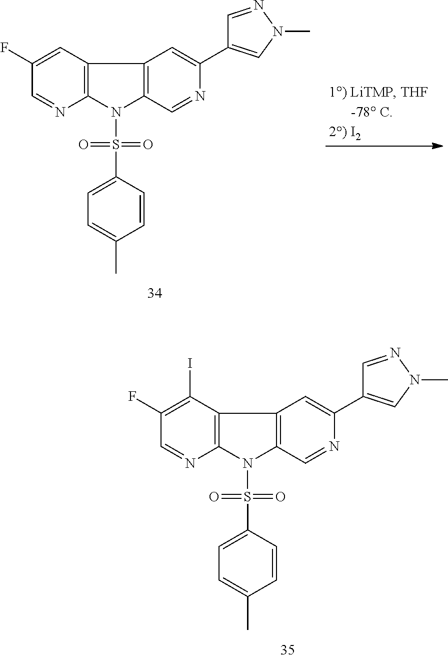

A mixture of 53 μl of 2,2,6,6-tetramethylpiperidine and 2 ml of tetrahydrofuran is cooled to −78° C., followed by addition of 100 μl of 2.7 N n-butyllithium in hexane. After 15 minutes, 75 mg of 3-fluoro-9-[(4-methylphenyl)sulfonyl]-6-(1-methyl-1H-pyrazol-4-yl)-9H-pyrrolo[2,3-b:5,4-c]c′dipyridine dissolved in 5 ml of tetrahydrofuran are added to the reaction mixture and the resulting mixture is stirred at −78° C. for 1 hour, followed by addition of 72 mg of iodine dissolved in 2 ml of tetrahydrofuran. The reaction mixture is poured into a mixture of water and saturated aqueous ammonium chloride solution and then extracted with ethyl acetate. The organic phase is washed with aqueous sodium thiosulfate solution, dried over magnesium sulfate, filtered and then concentrated to dryness under reduced pressure. The residue is purified by chromatography on a column of silica, eluting with ethyl acetate, to give 60 mg of 3-fluoro-4-iodo-9-[(4-methylphenyl)sulfonyl]-6-(1-methyl-1H-pyrazol-4-yl)-9H-pyrrolo-[2,3-b:5,4-c′]dipyridine in the form of a yellow solid.

UPLC-MS-DAD-ELSD (LS): Tr (min)=1.37; [M+H]⁺: m/z 548; purity: 71%

### Example 36

### 3-fluoro-4-iodo-6-(1-methyl-1H-pyrazol-4-yl)-8H-pyrrolo[2,3-b:5,4-c′]dipyridine

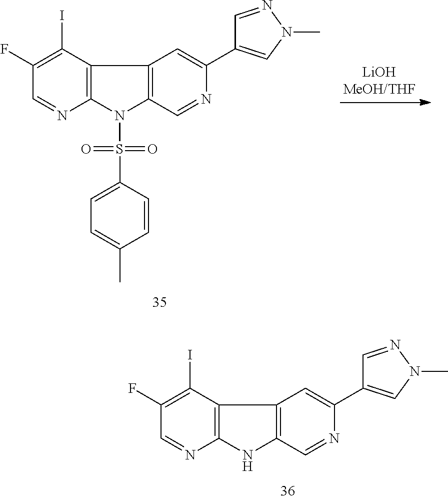

60 mg of 3-fluoro-4-iodo-9-[(4-methylphenyl)sulfonyl]-6-(1-methyl-1H-pyrazol-4-yl)-9H-pyrrolo[2,3-b:5,4-c′]dipyridine in 5 ml of methanol and 5 ml of tetrahydrofuran are placed in a round-bottomed flask and 60 mg of lithium hydroxide are then added. The reaction mixture is stirred for 18 hours at room temperature, followed by addition of a small amount of water and a few ml of aqueous ammonium chloride solution. The precipitate is filtered off by suction, washed with water, with ethyl acetate and with diethyl ether and then dried under vacuum to give 25 mg of 3-fluoro-4-iodo-6-(1-methyl-1H-pyrazol-4-yl)-9H-pyrrolo[2,3-b:5,4-c′]dipyridine.

1H NMR (400 MHz, DMSO-d6): ppm 3.94 (s, 3H) 8.03 (s, 1H) 8.34 (s, 1H) 8.59 (s, 1H) 8.83 (s, 1H) 8.99 (s, 1H) 12.73 (br. s., 1H)

### Example 37

### N-[3-(dimethylamino)ethyl]-4-[3-fluoro-6-(1-methy H-pyrazol-4-yl)-9H-pyrrolo[2,3-b:5,4-c′]dipyridin-4-yl]benzamide

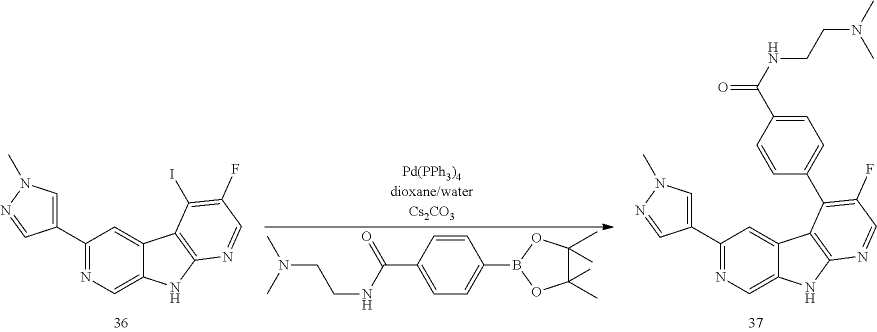

55 mg of 3-fluoro-4 iodo-6-(1-methyl-1H-pyrazol-4-yl)-9H-pyrrolo[2,3-b:5,4-c]dipyridine, 16 mg of tetrakis(triphenylphosphine)palladium(0), 134 mg of N-(2-dimethylaminoethyl)-4-(4,4,5,5-tetramethyl-1,3,2-dioxaborolan-2-yl)benzamide, 68 mg of caesium carbonate in 2 ml of 1,4-dioxane and 500 μl of water are placed in a reactor, and the tube is sealed and subjected to microwave irradiation at 120° C. for 1 hour. The reaction mixture is diluted with ethyl acetate and water, and the yellow precipitate formed is then filtered off by vacuum suction and washed with ethyl acetate, water and diethyl ether to give 45 mg N-[3-(dimethylamino)ethyl]-4-[3-fluoro-6-(1-methyl-1H-pyrazol-4-yl)-9H-pyrrolo[2,3-b:5,4-c′]dipyridin-4-yl]benzamide in the form of a yellow solid.

UPLC-MS-DAD-ELSD: Tr (min)=0.38; [M+H]⁺: m/z 458; [M−H]⁻: m/z 456; purity: 98%

1H NMR (400 MHz, DMSO-d6): δ ppm 2.23 (s, 6H) 2.46-2.49 (m, 2H) 3.45 (q, J=6.6 Hz, 2H) 3.85 (s, 3H) 7.29 (d, J=1.2 Hz, 1H) 7.54 (d, J=0.7 Hz, 1H) 7.82 (d, J=7.8 Hz, 2H) 7.92 (s, 1H) 8.14 (d, J=8.3 Hz, 2H) 8.61 (t, J=5.5 Hz, 1H) 871 (d, J=2.4 Hz, 1H) 8.89 (d, J=1.0 Hz, 1H) 12.36 (br. s., 1H)

### Example 38

### N-[2-(dimethylamino)propyl]-4-[3-fluoro-6-(1-methyl-1H-pyrazol-4-yl)-9H-pyrrolo[2,3-b:5,4-c′]dipyridin-4-yl]benzamide

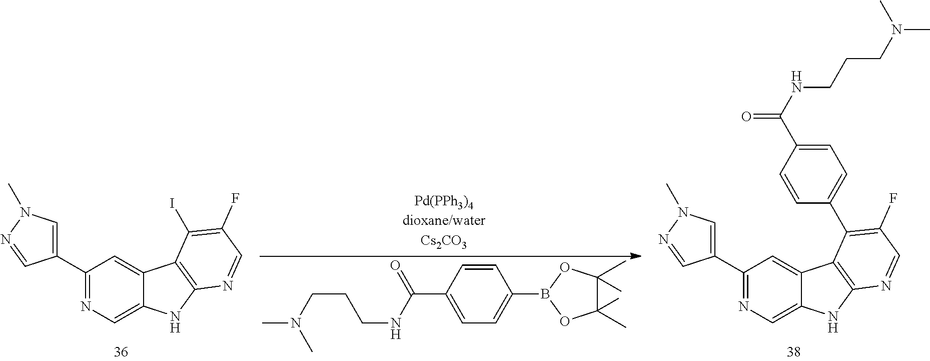

In a manner similar to that of Example 37, starting with 55 mg of product of Example 36, 35 mg of N-[2-(dimethylamino)propyl]-4-[3-fluoro-6-(1-methyl-1H-pyrazol-4-yl)-9H-pyrrolo[2,3-b:5,4-c′]dipyridin-4-yl]benzamide are obtained in the form of a pale yellow solid.

UPLC-MS-DAD-ELSD: Tr (min)=0.39; [M+H]⁺: m/z 472; purity: 95%

1H NMR (400 MHz, DMSO-d6): δ ppm 1.73 (quint., J=7.0 Hz, 2H) 2.17 (s, 6H) 2.32 (t, J=7.0 Hz, 2H) 3.38 (q, J=6.4 Hz, 2H) 3.84 (s, 3H) 7.30 (s, 1H) 7.54 (s, 1H) 781 (d, J=8.1 Hz, 2H) 7.92 (s, 1H) 8.14 (d, J=8.1 Hz, 2H) 8.68-8.76 (m, 2H) 8.89 (s, 1H) 12.37 (br. s., 1H)

### Example 39

### 5′-chloro-2′,4-dimethoxy-3,4′-bipyridin-2-amine

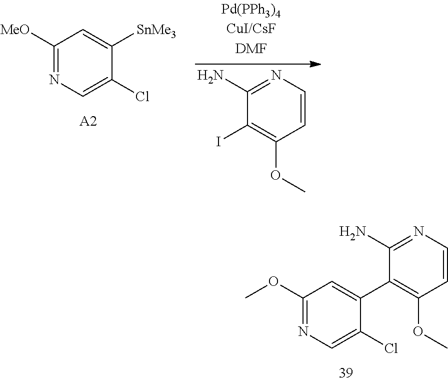

368 mg of 5-chloro-2-methoxy-4-(trimethylstannyl)pyridine B, 250 mg of 3-iodo-4-methoxypyridin-2-ylamine, 304 mg of caesium fluoride and 38 mg copper iodide in 2 ml of dimethylformamide are placed in a tube, 116 mg of tetrakis(triphenylphosphine)palladium(0) and 2 ml of dimethylformamide are then added, and the tube is sealed and subjected to microwave irradiation at 125° C. for 2 hours. The reaction mixture is filtered through Celite, rinsed with 10 ml of ethyl acetate and then washed with twice 10 ml of water. After separation of the phases by settling, the organic phase is dried over magnesium sulfate, filtered and then concentrated to dryness under reduced pressure. The residue is purified by chromatography on a column of silica, eluting with a 50/50 to 0/100 heptane/ethyl acetate mixture to give 125 mg of 5′-chloro-2′,4-dimethoxy-3,4′-bipyridin-2-amine in the form of a white solid.

UPLC-MS-DAD-ELSD: Tr (min)=0.44; [M+H]⁺: m/z 266; purity: 98%

1H NMR (400 MHz, DMSO-d6): δ ppm 3.68 (s, 3H) 3.87 (s, 3H) 5.40 (s, 2H) 6.42 (d, J=5.9 Hz, 1H) 6.72 (d, J=0.5 Hz, 1H) 7.94 (d, J=5.9 Hz, 1H) 8.28 (d, J=0.5 Hz, 1H)

### Example 40

### 4,6-dimethoxy-9H-pyrrolo[2,3-b:5,4-c′]dipyridine

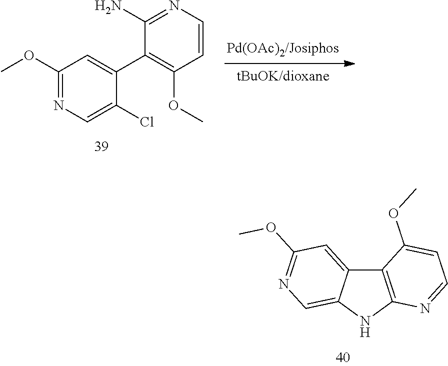

10.3 mg of (R)-(−)-1-[(S)-2-(dicyclohexylphosphino)ferrocenyl]ethyldi-tert-butylphosphine and 3.8 mg of palladium (II)acetate in 0.35 ml of anhydrous 1,4-dioxane are placed in a 2 ml tube under an argon atmosphere, and the mixture is stirred for 10 minutes at 35° C.

45 mg of 5′-chloro-2′,4-dimethoxy-3,4′-bipyridin-2-amine and 38 mg of potassium tert-butoxide in 0.35 ml of anhydrous 1,4-dioxane are placed in a 2 ml reactor under argon, followed by addition of the solution prepared previously and 0.20 ml of 1,4-dioxane, and the tube is sealed and subjected to microwave irradiation for 1 hour at 130° C. The reaction mixture is diluted with a 90/10 dichloromethane/methanol mixture and then filtered. After concentrating under reduced pressure, the residue is purified by chromatography on a column of silica, eluting with a 98/2 to 94/6 dichloromethane/methanol mixture to give 28.5 mg of 4,6-dimethoxy-9H-pyrrolo[2,3-b:5,4-c′]dipyridine in the form of a yellow solid.

UPLC-MS-DAD-ELSD: Tr (min)=0.40; [M+H]⁺: m/z 230; purity: 95%

1H NMR (400 MHz, DMSO-d6): δ ppm 3.89 (s, 3H) 4.09 (s, 3H) 6.85 (d, J=5.9 Hz, 1H) 7.30 (d, J=1.0 Hz, 1H) 8.39-8.42 (m, 2H) 11.70 (br. s., 1H)

### Example 41

### 9H-pyrrolo[2,3-b:5,4-c′]dipyridine-4,6-diol hydrochloride

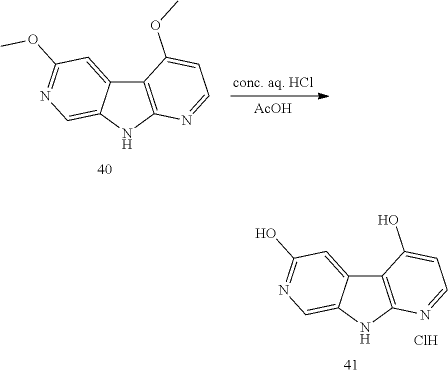

1.52 g of 4,6-dimethoxy-9H-pyrrolo[2,3-b:5,4-c′]dipyridine in 22.1 ml of acetic acid and 7.3 ml of 37% hydrochloric acid solution are placed in a 20 ml reactor, and the tube is sealed and subjected to microwave irradiation for 2 hours at 140° C. After concentrating the reaction mixture, the solid obtained is slurried in twice 25 ml of diethyl ether and then dried under reduced pressure for 18 hours to give 1.72 g of 9H-pyrrolo[2,3-b:5,4-c′]dipyridine-4,6-diol hydrochloride in the form of a dark beige solid.

UPLC-MS-DAD-ELSD: Tr (min)=0.14; [M+H]⁺: m/z 202; [M−H]⁻: m/z 200; purity: 98%

1H NMR (400 MHz, DMSO-d6): δ ppm 6.48 (m, 1H) 7.62 (s, 1H) 8.06 (d, J=7.1 Hz, 1H) 8.34 (s, 1H) 12.48 (br. s., 1H)

### Example 42

### 9H-pyrrolo[2,3-b:5,4-c′]dipyridine-4,6-diyl bis(trifluoromethanesulfonate)

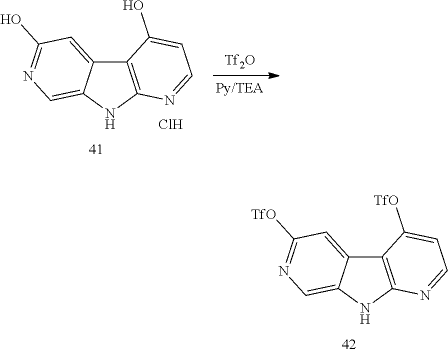

A mixture of 1.72 g of 9H-pyrrolo[2,3-b:5,4-c′]dipyridine-4,6-diol hydrochloride in 35 ml of pyridine and 9.1 ml of triethylamine is cooled to 5° C., followed by addition of 2.8 ml of anhydrous trifluoromethanesulfonic acid. The reaction mixture is stirred at 0-5° C. for 1 hour and then poured into a mixture of 200 ml of water and 50 ml of saturated aqueous sodium chloride solution and extracted with 250 ml of ethyl acetate. After separation of the phases by settling, the aqueous phase is extracted with 200 ml of ethyl acetate and the organic phases are then combined and concentrated under vacuum. The residue is taken up in a mixture of 100 ml of an 80/20 dichloromethane/ethyl acetate mixture, 6.0 g of silica are added, and the resulting mixture is concentrated under reduced pressure. The solid deposit formed is purified by chromatography on a column of silica, eluting with a 100/0 to 80/20 dichloromethane/ethyl acetate mixture to give 124 mg of 9H-pyrrolo[2,3-b:5,4-c′]dipyridine-4,6-diylbis(trifluoromethanesulfonate) in the form of a rust-coloured solid.

UPLC-MS-DAD-ELSD: Tr (min)=4.81; [M+H]⁺: m/z 466; [M−H]⁻: m/z 464; purity: 98%

1H NMR (400 MHz, DMSO-d6): δ ppm 7.59 (d, J=5.6 Hz, 1H) 7.96 (s, 1H) 8.88 (d, J=0.7 Hz, 1H) 8.89 (d, J=5.6 Hz, 1H) 13.32 (br. s., 1H)

### Example 43

### 4-(4-{[2-(dimethylamino)ethyl]carbamoyl}phenyl)-9H-pyrrolo[2,3-b:5,4-c′]dipyridin-6-yl trifluoromethanesulfonate

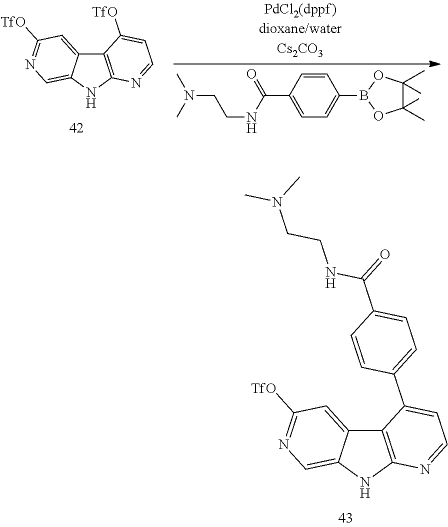

124 mg of 9H-pyrrolo[2,3-b:5,4-c′]dipyridine-4,6-diyl bis(trifluoromethanesulfonate), mg of N-(2-dimethylaminoethyl)-4-(4,4,5,5-tetramethyl-1,3,2-dioxaborolan-2-yl)benzamide and 130 mg of caesium carbonate in 1.2 ml of 1,4-dioxane are placed in a reactor, followed by addition, under argon, of 19 mg of 1,1′-bis(diphenylphosphino)ferrocenedichloropalladium(II) and 0.12 ml of water, and the tube is sealed and subjected to microwave irradiation at 140° C. for 15 minutes. The reaction mixture is diluted with 10 ml of ethyl acetate and washed with 10 ml of water. After separation of the phases by settling, the aqueous phase is extracted with 10 ml of ethyl acetate and the organic phases are then combined, dried over magnesium sulfate, filtered and then concentrated under vacuum. The residue is taken up in a mixture of 30 ml of a 90/10 dichloromethane/methanol mixture, 600 mg of silica are added and the resulting mixture is concentrated under reduced pressure. The solid deposit formed is purified by chromatography on a column of silica, eluting with a 100/0 to 90/10 dichloromethane/methanol mixture to give 56 mg of 4-(4-{[2-(dimethylamino)ethyl]carbamoyl}phenyl)-9H-pyrrolo[2,3-b:5,4-c′]dipyridin-6-yl trifluoromethanesulfonate the form of a beige-coloured solid.

UPLC-MS-DAD-ELSD: Tr (min)=4.81; [M+H]⁺: m/z 466; [M−H]⁻: m/z 464; purity: 98%

1H NMR (400 MHz, DMSO-d6): 6 ppm 2.36 (s, 6H) 2.62-2.69 (m, 2H) 3.48 (q, J=6.5 Hz, 2H) 7.33-7.38 (m, 2H) 7.85 (d, J=8.3 Hz, 2H) 8.12 (d, J=8.3 Hz, 2H) 8.61-8.07 (m, 1H) 8.74-8.78 (m, 2H) 12.86 (br. s., 1H)

### Example 44

### N-[2-(dimethylamino)ethyl]-4-[6-(1-ethyl-1H-pyrazol-4-yl)-9H-pyrrolo[2,3-b:5,4-c′]dipyridin-4-yl]benzamide

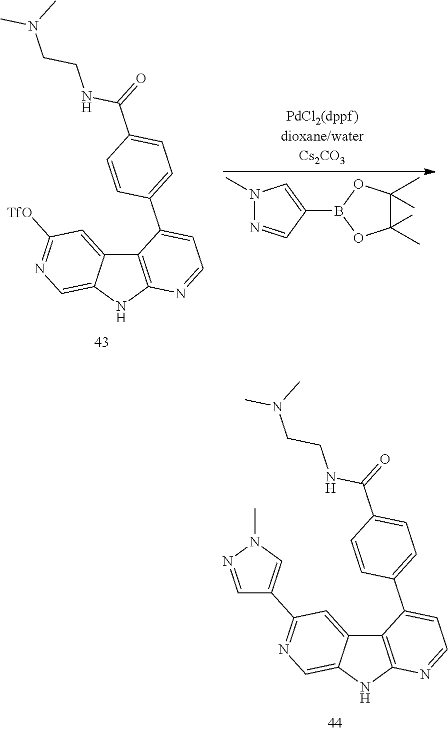

53 mg of 4-(4-{[2-(dimethylamino)ethyl]carbamoyl}phenyl)-9H-pyrrolo[2,3-b:5,4-c′]dipyridin-6-yl trifluoromethanesulfonate, 33 mg of 1-methyl-4-(4,4,5,5-tetramethyl-1,3,2-dioxaborolan-2-yl)-1H-pyrazole and 51 mg of caesium carbonate in 0.8 ml of 1,4-dioxane are placed in a reactor, followed by addition, under argon, of 8 mg of 1,1′-bis(diphenylphosphino)ferrocenedichloropalladium(II) and 85 μl of water, and the tube is sealed and subjected to microwave irradiation at 140° C. for 30 minutes. 21 mg of 1-methyl-4-(4,4,5,5-tetramethyl-1,3,2-dioxaborolan-2-yl)-1H-pyrazole and 8 mg of 1,1′-bis(diphenylphosphino)ferrocenedichloropalladium(II) are added to the reaction mixture, and the mixture is again subjected to microwave irradiation at 140° C. for 30 minutes. The reaction mixture is diluted with 10 ml of ethyl acetate and treated with 10 ml of water. After separation of the phases by settling, the aqueous phase is extracted with 10 ml of ethyl acetate and the organic phases are then combined, dried over magnesium sulfate, filtered and then concentrated under vacuum. The residue is purified by chromatography on a column of silica, eluting with a 100/0 to 88/12 dichloromethane/methanol mixture; the solid obtained is washed with 1 ml of diethyl ether to give 7 mg of N-[2-(dimethylamino)ethyl]-4-[6-(1-methyl-1H-pyrazol-4-yl)-9H-pyrrolo[2,3-b:5,4-c′]dipyridin-4-yl]benzamide the form of a beige-coloured solid.

UPLC-MS-DAD-ELSD: Tr (min)=2.01; [M+H]⁺: m/z 440; [M−H]⁻: m/z 438; purity: 90%

1H NMR (500 MHz, DMSO-d6): δ ppm 2.22 (s, 6H) 2.44-2.49 (m, 2H) 3.45 (q, J=6.6 Hz, 2H) 3.86 (s, 3H) 7.26 (d, J=4.9 Hz, 1H) 7.61 (s, 1H) 7.63 (s, 1H) 7.86 (d, J=8.2 Hz, 2H) 7.98 (s, 1H) 8.13 (d, J=8.2 Hz, 2H) 8.60 (t, J=5.4 Hz, 1H) 8.64 (d, J=4.9 Hz, 1H) 8.89 (s, 1H) 12.32 (br. s., 1H)

### Examples 45 to 82

### General Procedure for Examples 45 to 82

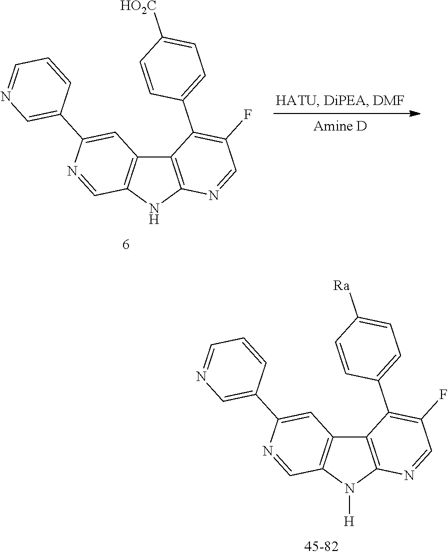

A solution containing the carboxylic acid 6 (2.51 g, 5.6 mmol), HATU (6.16 mmol 1.1 eq.) and diisopropylethylamine (DIPEA, 7 mmol, 1.25 eq.) in 80 ml of DMF is prepared. 2 ml of this solution are taken and then added to 0.175 mmol (1.25 eq.) of the amine D in a tube. If the amine D is in hydrochloride form, 1.25 eq. of DIPEA are added per molecule of HCl present in D. The tubes are closed and the various mixtures are then heated overnight with stirring at 50° C. After cooling, 0.1 ml of TFA is added. After filtration, the expected products 45-83, present in the various filtrates, are purified by preparative HPLC.

Analytical Conditions:

HPLC: YMC-Pack Jshere H80 33×2.1; 4μ; H₂O+0.0.5% TFA/CH₃CN: 98/2 (1 min) to 5/95 (5 min). MS: Waters LOT classic TOF-MS, 8-channel Mux, 0.15 s scantime for mass 100-1500

Or

HPLC: Waters UPLC BEH C18XBridge C18 50×2.1 mm; 1.7μ; H₂O+0.1% HCOOH/CH₃CN+0.08% HCOOH: 95/5 (0 min) to 5/95 (1.1 min) to 5/95 (1.7 min) to 95/5 (1.8 min) to 95/5 (2 min). MS: Waters SQQ Single Quadrupole, 0.5 s scantime for mass 120-1200.

The structures obtained via this process are described in Table 3

TABLE 3

| | | | Retention | [M + H⁺] |
|---|---|---|---|---|
| Ex. No. | Structure | Name | time (min) | observed |
| 45 | 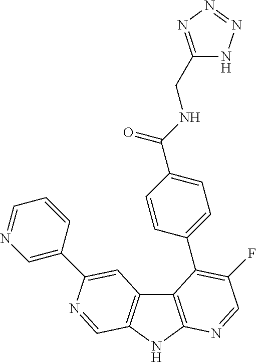 | 4-[3-fluoro-6-(pyridin-3-yl)- 9H-pyrrolo[2,3-b:5,4-c′]di- pyridin-4-yl]-N-(1H-tetrazol- 5-ylmethyl)benzamide | 2.27 | 466.23 |
| 46 | 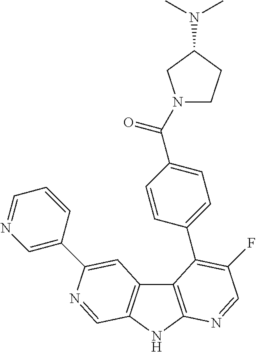 | [(3R)-3-(dimethylamino) pyrrolidin-1-yl]{4-[3-fluoro-6- (pyridin-3-yl)-9H-pyrrolo[2,3- b:5,4-c′]dipyridin-4-yl]- phenyl}methanone | 2.12 | 481.3 |
| 47 | 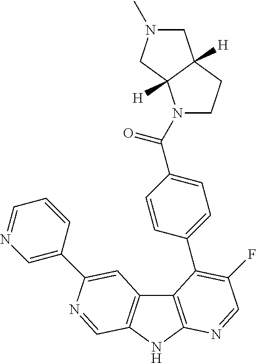 | {4-[3-fluoro-6-(pyridin-3-yl)- 9H-pyrrolo[2,3-b:5,4-c′]di- pyridin-4-yl]phenyl}- [(3aS,6aS)-5-methylhexa- hydropyrrolo[3,4-b]pyrrol- 1(2H)-yl]methanone | 2.18 | 493.31 |
| 48 | 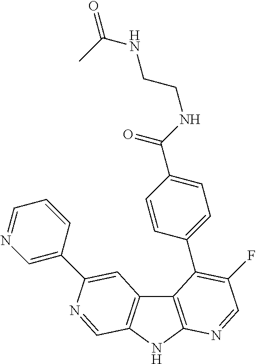 | N-[2-(acetylamino)ethyl]-4- [3-fluoro-6-(pyridin-3-yl)-9H- pyrrolo[2,3-b:5,4-c′]dipyridin- 4-yl]benzamide | 2.22 | 469.24 |
| 49 | 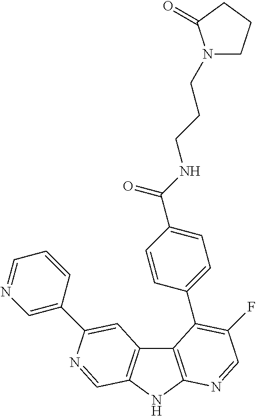 | 4-[3-fiuoro-6-(pyridin-3-yl)- 9H-pyrrolo[2,3-b:5,4-c′]di- pyridin-4-yl]-N-[3-(2-oxopyr- rolidin-1-yl)propyl]benzamide | 2.37 | 509.27 |
| 50 | 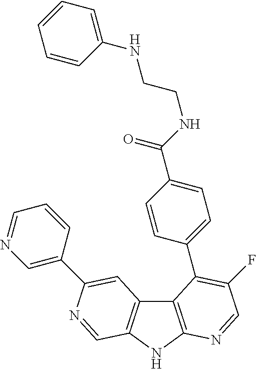 | 4-[3-fluoro-6-(pyridin-3-yl)- 9H-pyrrolo[2,3-b:5,4-c′]di- pyridin-4-yl]-N-[2-(phenyl- amino)ethyl]benzamide | 2.50 | 503.27 |
| 51 | 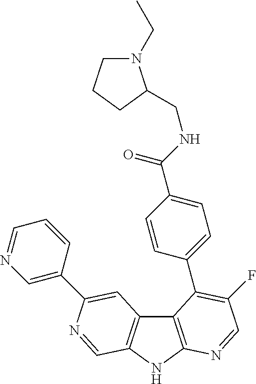 | N-[(1-ethylpyrrolidin-2-yl)- methyl]-4-[3-fluoro-6- (pyridin-3-yl)-9H-pyrrolo[2,3- b:5,4-c′]dipyridin-4-yl] benzamide | 2.17 | 495.31 |
| 52 | 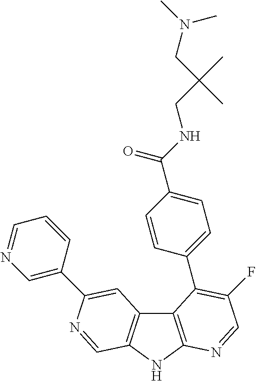 | N-[3-(dimethylamino)-2,2- dimethylpropyl]-4-[3-fluoro-6- (pyridin-3-yl)-9H-pyrrolo[2,3- b:5,4-c′]dipyridin-4-yl] benzamide | 2.22 | 497.32 |
| 53 | 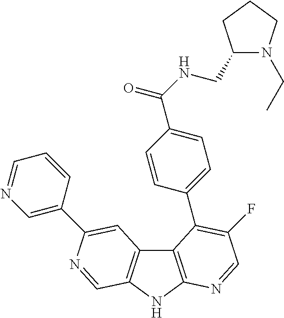 | N-{[(2S)-1-ethylpyrrolidin-2- yl]methyl}-4-[3-fluoro-6- (pyridin-3-yl)-9H-pyrrolo[2,3- b:5,4-c′]dipyridin-4-yl] benzamide | 2.22 | 495.31 |
| 54 | 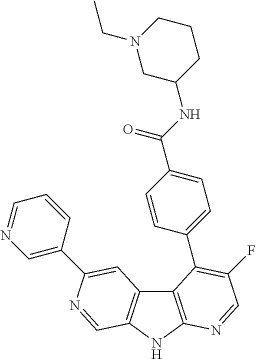 | N-(1-ethylpiperidin-3-yl)-4-[3- fluoro-6-(pyridin-3-yl)-9H- pyrrolo[2,3-b:5,4-c′]dipyridin- 4-yl]benzamide | 2.17 | 495.31 |
| 55 | 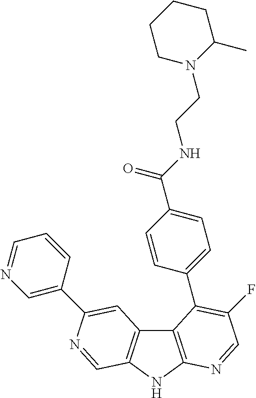 | 4-[3-fluoro-6-(pyridin-3-yl)- 9H-pyrrolo[2,3-b:5,4-c′]di- pyridin-4-yl]-N-[2-(2-methyl- piperidin-1-yl)ethyl] benzamide | 2.29 | 509.33 |
| 56 | 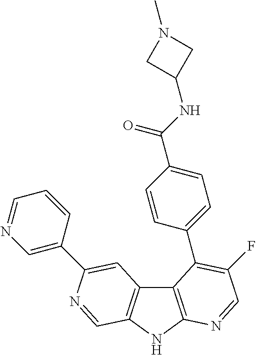 | 4-[3-fluoro-6-(pyridin-3-yl)- 9H-pyrrolo[2,3-b:5,4-c′]di- pyridin-4-yl]-N-(1-methyl- azetidin-3-yl)benzamide | 0.77 | 453.25 |
| 57 | 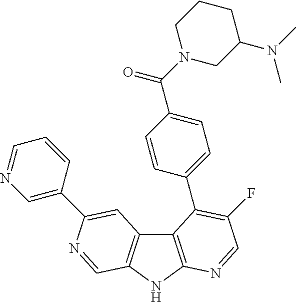 | [3-(dimethylamino)piperidin- 1-yl]{4-[3-fluoro-6-(pyridin-3- yl)-9H-pyrrolo[2,3-b:5,4-c′]di- pyridin-4-yl]phenyl}methan- one | 2.17 | 495.32 |
| 58 | 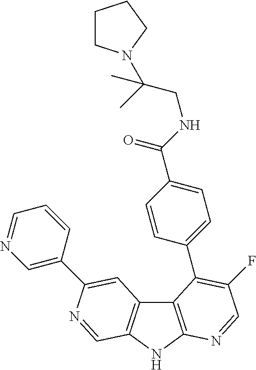 | 4-[3-fluoro-6-(pyridin-3-yl)- 9H-pyrrolo[2,3-b:5,4-c′]di- pyridin-4-yl]-N-[2-methyl-2- (pyrrolidin-1-yl)propyl] benzamide | 2.25 | 509.33 |
| 59 | 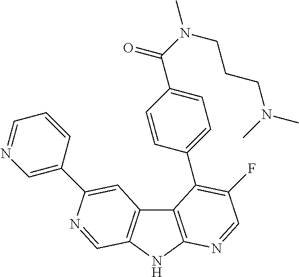 | N-[3-(dimethylamino)propyl]- 4-[3-fluoro-6-(pyridin-3-yl)- 9H-pyrrolo[2,3-b:5,4-c′]di- pyridin-4-yl]-N-methylbenz- amide | 2.15 | 483.31 |
| 60 | 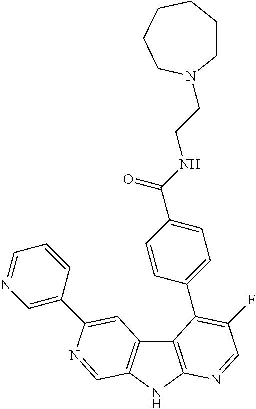 | N-[2-(azepan-1-yl)ethyl]-4- [3-fluoro-6-(pyridin-3-yl)-9H- pyrrolo[2,3-b:5,4-c′]dipyridin- 4-yl]benzamide | 2.29 | 509.33 |
| 61 | 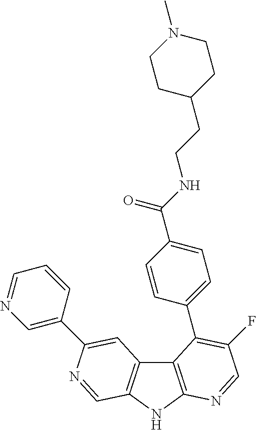 | 4-[3-fluoro-6-(pyridin-3-yl)- 9H-pyrrolo[2,3-b:5,4-c′]di- pyridin-4-yl]-N-[2-(1-methyl- piperidin-4-yl)ethyl] benzamide | 2.23 | 509.33 |
| 62 | 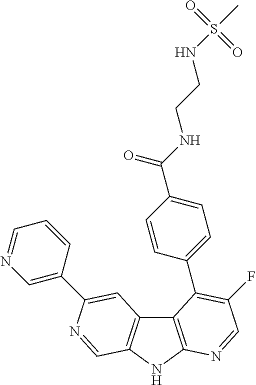 | 4-[3-fluoro-6-(pyridin-3-yl)- 9H-pyrrolo[2,3-b:5,4-c′]di- pyridin-4-yl]-N-{2-[(methyl- sulfonyl)amino]ethyl} benzamide | 2.32 | 505.24 |
| 63 | 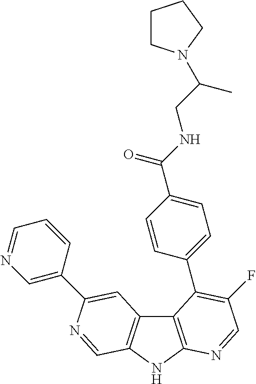 | 4-[3-fluoro-6-(pyridin-3-yl)- 9H-pyrrolo[2,3-b:5,4-c′]di- pyridin-4-yl]-N-[2-(pyrrolidin- 1-yl)propyl]benzamide | 2.22 | 495.33 |
| 64 | 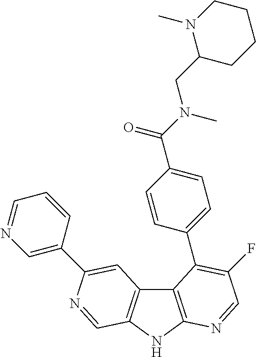 | 4-[3-fiuoro-6-(pyridin-3-yl)- 9H-pyrrolo[2,3-b:5,4-c′]di- pyridin-4-yl]-N-methyl-N-[(1- methylpiperidin-2-yl)methyl]- benzamide | 2.20 | 509.35 |
| 65 | 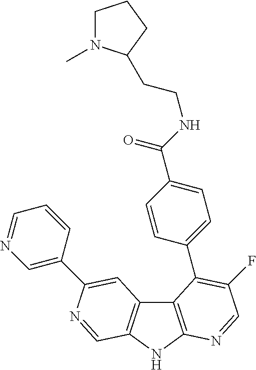 | 4-[3-fluoro-6-(pyridin-3-yl)- 9H-pyrrolo[2,3-b:5,4-c′]di- pyridin-4-yl]-N-[2-(1- methylpyrrolidin-2-yl)ethyl]- benzamide | 2.20 | 495.3 |
| 66 | 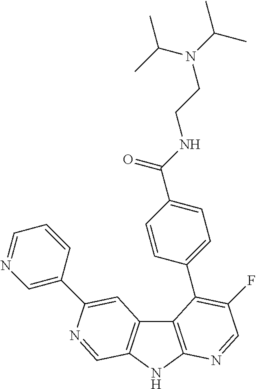 | N-[2-(dipropan-2-ylamino)- ethyl]-4-[3-fluoro-6-(pyridin- 3-yl)-9H-pyrrolo[2,3-b:5,4- c′]dipyridin-4-yl]benzamide | 2.29 | 511.33 |
| 67 | 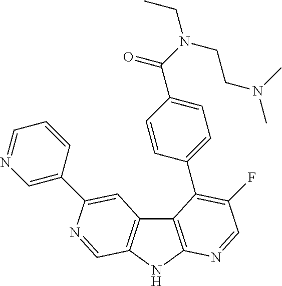 | N-[2-(dimethylamino)ethyl]- N-ethyl-4-[3-fluoro-6- (pyridin-3-yl)-9H-pyrrolo[2,3- b:5,4-c′]dipyridin-4-yl] benzamide | 2.18 | 483.3 |
| 68 | 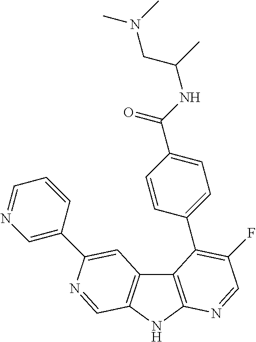 | N-[1-(dimethylamino)propan- 2-yl]-4-[3-fluoro-6-(pyridin-3- yl)-9H-pyrrolo[2,3-b:5,4c′]di- pyridin-4-yl]benzamide | 0.8 | 469.26 |
| 69 | 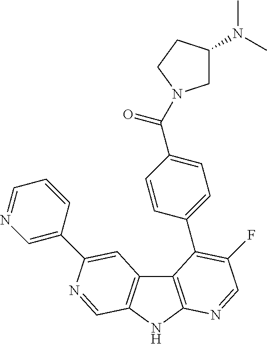 | [(3S)-3-(dimethylamino)pyr- rolidin-1-yl]{4-[3-fluoro-6- (pyridin-3-yl)-9H-pyrrolo[2,3- b:5,4-c′]dipyridin-4-yl]- phenyl}methanone | 0.76 | 481.29 |
| 70 | 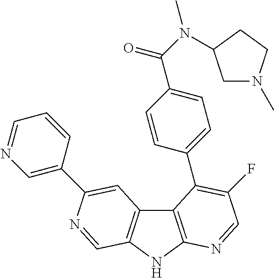 | 4-[3-fluoro-6-(pyridin-3-yl)- 9H-pyrrolo[2,3-b:5,4-c′]di- pyridin-4-yl]-N-methyl-N-(1- methylpyrrolidin-3-yl) benzamide | 2.14 | 481.29 |
| 71 | 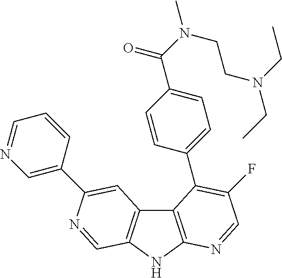 | N-[2-(diethylamino)ethyl]-4- [3-fluoro-6-(pyridin-3-yl)-9H- pyrrolo[2,3-b:5,4-c′]dipyridin- 4-yl]-N-methylbenzamide | 2.18 | 497.33 |
| 72 | 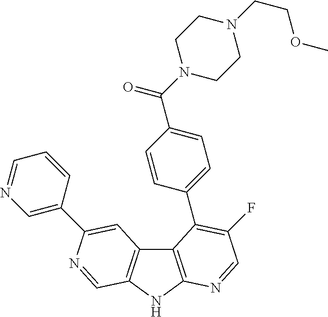 | {4-[3-fluoro-6-(pyridin-3-yl)- 9H-pyrrolo[2,3-b:5,4-c′]di- pyridin-4-yl]phenyl}[4-(2- methoxyethyl)piperazin-1- yl]methanone | 2.12 | 511.32 |
| 73 | 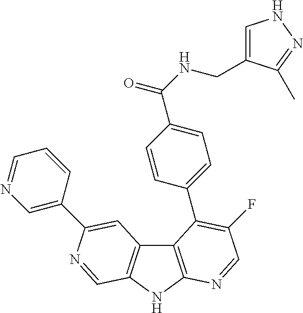 | 4-[3-fluoro-6-(pyridin-3-yl)- 9H-pyrrolo[2,3-b:5,4-c′]di- pyridin-4-yl]-N-[(3-methyl- 1H-pyrazol-4-yl)methyl] benzamide | 2.32 | 478.26 |
| 74 | 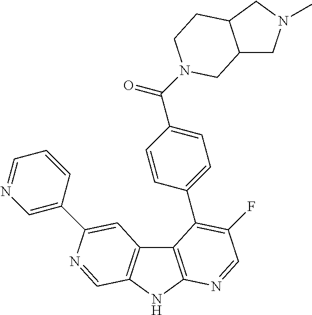 | {4-[3-fluoro-6-(pyridin-3-yl)- 9H-pyrrolo[2,3-b:5,4-c′]di- pyridin-4-yl]phenyl}(2- methyloctahydro-5H-pyrrolo- [3,4-c]pyridin-5-yl) methanone | 0.79 | 507.23 |
| 75 | 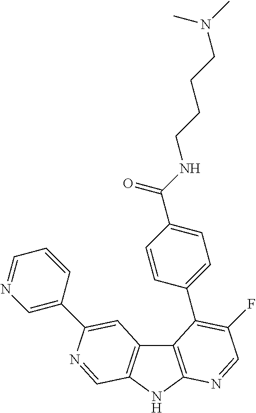 | N-[4-(dimethylamino)butyl]- 4-[3-fluoro-6-(pyridin-3-yl)- 9H-pyrrolo[2,3-b:5,4-c′]di- pyridin-4-yl]benzamide | 2.20 | 483.32 |
| 76 | 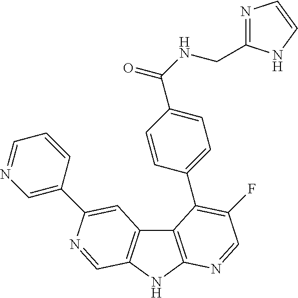 | 4-[3-fluoro-6-(pyridin-3-yl)- 9H-pyrrolo[2,3-b:5,4-c′]di- pyridin-4-yl]-N-(1H-imidazol- 2-ylmethyl)benzamide | 2.14 | 464.27 |
| 77 | 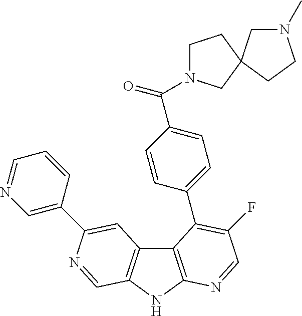 | {4-[3-fluoro-6-(pyridin-3-yl)- 9H-pyrrolo[2,3-b:5,4-c′]di- pyridin-4-yl]phenyl}(7- methyl-2,7-diazaspiro[4.4]- non-2-yl)methanone | 2.15 | 507.33 |
| 78 | 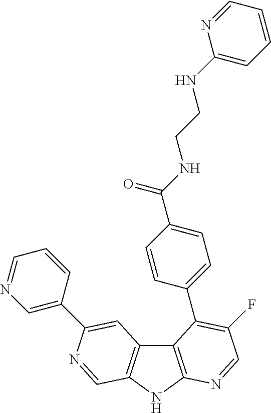 | 4-[3-fluoro-6-(pyridin-3-yl)- 9H-pyrrolo[2,3-b:5,4-c′]di- pyridin-4-yl]-N-[2-(pyridin-2- ylamino)ethyl]benzamide | 0.83 | 504.26 |
| 79 | 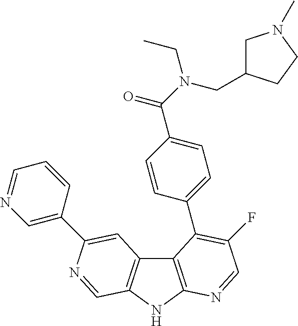 | N-ethyl-4-[3-fluoro-6- (pyridin-3-yl)-9H-pyrrolo[2,3- b:5,4-c′]dipyridin-4-yl]-N-[(1- methylpyrrolidin-3-yl)methyl]- benzamide | 2.22 | 509.34 |
| 80 | 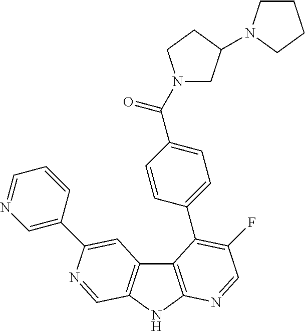 | 1.3′-bipyrrolidin-1′-yl{4-[3- fluoro-6-(pyridin-3-yl)-9H- pyrrolo[2,3-b:5,4-c′]dipyridin- 4-yl]phenyl}methanone | 2.14 | 507.33 |
| 81 | 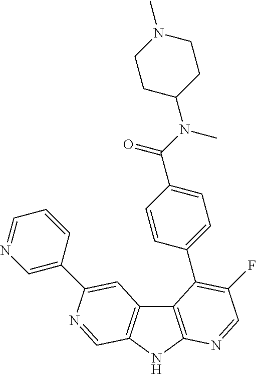 | 4-[3-fluoro-6-(pyridin-3-yl)- 9H-pyrrolo[2,3-b:5,4-c′]di- pyridin-4-yl]-N-methyl-N-(1- methylpiperidin-4-yl) benzamide | 2.15 | 495.34 |
| 82 | 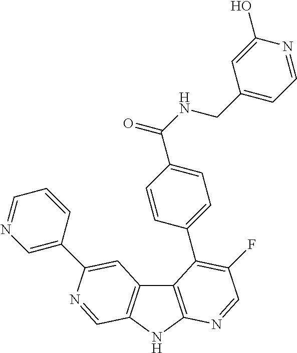 | 4-[3-fluoro-6-(pyridin-3-yl)- 9H-pyrrolo[2,3-b:5,4-c′]di- pyridin-4-yl]-N-[(2-hydroxy- pyridin-4-yl)methyl] benzamide | 2.32 | 491.27 |

### Examples 83 to 86

### General Procedure for Examples 83 to 86

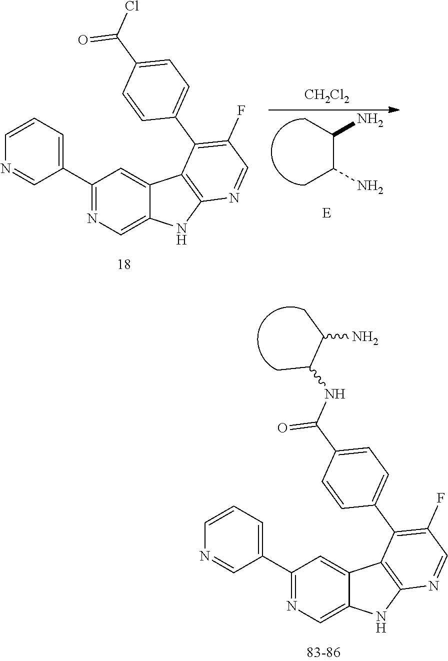

4-[3-Fluoro-6-(pyridin-3-yl)-9H-pyrrolo[2,3-b:5,4-c′]dipyridin-4-yl]benzoyl chloride 18 (1 eq.) is suspended in dichloromethane (50 mL per 1 mmol of 18). 5 to 10 equivalents of diamine E are dissolved in dichloromethane (same volume used as for placing 18 in suspension) in a dry round-bottomed flask under an argon atmosphere. The suspension of 18 is then added to the solution containing E with stirring at room temperature. The mixture is stirred for 2 to 20 hours. Methanol is then added until the precipitate disappears, and silica (10 mg per 1 mg of 18) is then added. The solvents are evaporated off under reduced pressure and the product is recovered by chromatography on silica gel (eluent: CH₂Cl₂/MeOH or CH₂Cl₂/2N NH₃ in MeOH: gradient from 100/0 to 85/15).

Examples 83 to 86 obtained via this method are described in Table 4.

TABLE 4

| | | | Retention | [M + H⁺] |
|---|---|---|---|---|
| Ex. No. | Structure | Name | time (min) | observed |
| 83 | 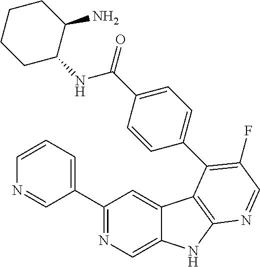 | N-[(1R,2R)-2-amino- cyclohexyl]-4-[3-fluoro- 6-(pyridin-3-yl)-9H- pyrrolo[2,3-b:5,4-c′]di- pyridin-4-yl]benzamide | 0.51 | 481.36 |
| 84 | 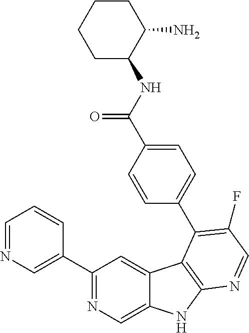 | N-[(1S,2S)-2-amino- cyclohexyl]-4-[3-fluoro- 6-(pyridin-3-yl)-9H- pyrrolo[2,3-b:5,4-c′]di- pyridin-4-yl]benzamide | 2.51 | 481.15 |
| 85 | 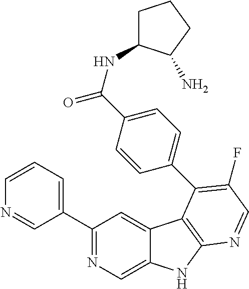 | N-[(1S,2S)-2-amino- cyclopentyl]-4-[3-fluoro- 6-(pyridin-3-yl)-9H- pyrrolo[2,3-b:5,4-c′]di- pyridin-4-yl]benzamide | 0.46 | 467.37 |
| 86 | 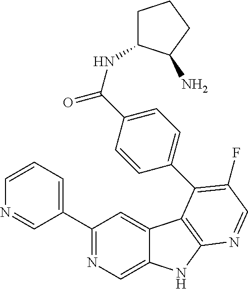 | N-[(1R,2R)-2-amino- cyclopentyl]-4-[3-fluoro- 6-(pyridin-3-yl)-9H- pyrrolo[2,3-b:5,4-c′]di- pyridin-4-yl]benzamide | 0.46 | 467.38 |

### Examples 87 to 91

### Synthesis of 1-aminomethylcyclopropylamine

The synthesis of 1-aminomethylcyclopropylamine is performed via hydrogenation as described in J. Org. Chem. 1992, 57(22), 6071-6075.

### Synthesis of 2,2-difluoropropan-1,3-diamine

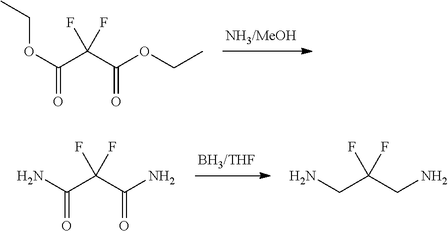

45 ml of ammoniacal methanol (7N) are added to 5 g (25 mmol) of ethyl difluoromalonate at 0° C. in a 250 mL three-necked flask. The mixture is stirred until it has warmed to room temperature, and is then left stirring overnight. The difluoromalonadiamide is recovered by evaporating the mixture under reduced pressure (3.35 g; 95% yield). 500 mg of this residue are placed in a microwave tube, and 17 mL of a boron hydride/tetrahydrofuran solution (1 M BH₃/THF in THF) are then added slowly at 0° C. After warming to room temperature, the mixture is left stirring until the evolution of gas has ceased. The tube is sealed and then heated by microwave for 30 minutes at 120° C. 5 ml of methanol are then added to the reaction medium, cooled beforehand to 0° C. The solvents are evaporated off under reduced pressure. The residue is taken up in 20 ml of methanol and then evaporated to dryness, this operation being repeated twice. 291 mg of 2,2-difluoropropan-1,3-diamine are obtained and are used in the following step without further purification.

### Synthesis of 2-trifluoromethylpropan-1,2-diamine

2-Trifluoromethylpropan-1,2-diamine is prepared racemically by following the synthesis described in J. Org. Chem. 2006, 71(18), 7075-7079. To obtain the racemic compound, the optically pure α-methylbenzylamine used in the publication is simply replaced with benzylamine in the first step of the process. The rest of the synthesis proceeds in accordance with the publication.

### General Procedure for Examples 87 to 91

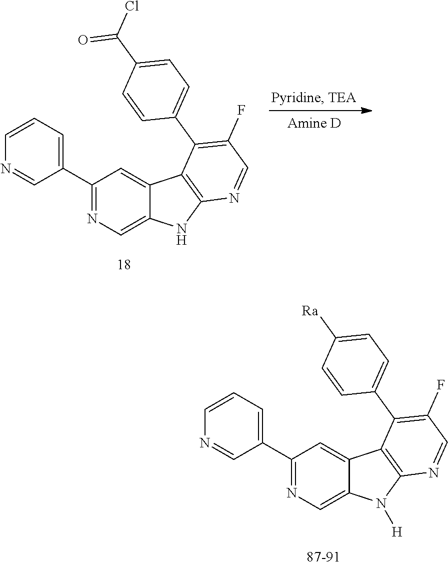

1.2 mmol (10 eq.) of amine D are dissolved in 0.5 ml of anhydrous pyridine (addition of triethylamine if the amine E is in hydrochloride form) in a dry round-bottomed flask under an argon atmosphere. 4-[3-Fluoro-6-(pyridin-3-yl)-9H-pyrrolo[2,3-b:5,4-a]dipyridin-4-yl]benzoyl chloride 18 (50 mg, 0.124 mmol, 1 eq.) is dissolved in 1 ml of pyridine. This solution is then added to the solution containing with stirring at room temperature. The mixture is left stirring for between 30 minutes and 2 hours, and if the reaction is incomplete after 2 hours, it may also be heated for 30 minutes by microwave at 140° C. The pyridine is then evaporated off under reduced pressure. The residue is dissolved in a CH₂Cl₂/MeOH mixture and 500 mg of silica are added. The solvents are evaporated off under reduced pressure and the product is recovered by chromatography on silica gel (eluent: CH₂Cl₂/MeOH or CH₂Cl₂/2N NH₃ in MeOH: gradient from 100/0 to 85/15).

Examples 87 to 91 obtained via this method are described in Table 5.

TABLE 5

| | | | Retention | [M + H⁺] |
|---|---|---|---|---|
| Ex. No. | Structure | Name | time (min) | observed |
| 87 | 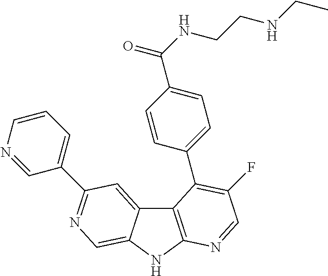 | N-[2-(ethylamino)ethyl]-4- [3-fluoro-6-(pyridin-3-yl)- 9H-pyrrolo[2,3-b:5,4-c′]di- pyridin-4-yl]benzamide | 0.41 | 455.16 |
| 88 | 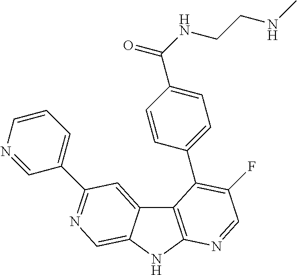 | 4-[3-fluoro-6-(pyridin-3-yl)- 9H-pyrrolo[2,3-b:5,4-c′]di- pyridin-4-yl]-N-[2-(methyl- amino)ethyl]benzamide | 0.40 | 441.14 |
| 89 | 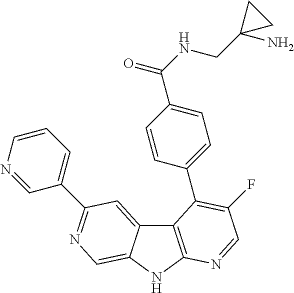 | N-[(1-aminocyclopropyl)- methyl]-4-[3-fluoro-6- (pyridin-3-yl)-9H-pyrrolo- [2,3-b:5,4-c′]dipyridin-4-yl]- benzamide | 0.42 | 453.17 |
| 90 | 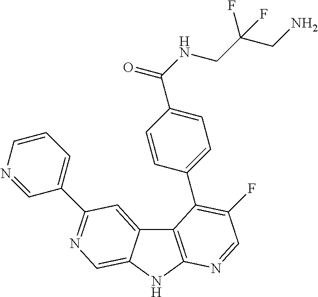 | N-(3-amino-2,2-difluoro- propyl)-4-[3-fluoro-6- (pyridin-3-yl)-9H-pyrrolo- [2,3-b:5,4-c′]dipyridin-4-yl]- benzamide | 2.24 | 477.13 |
| 91 | 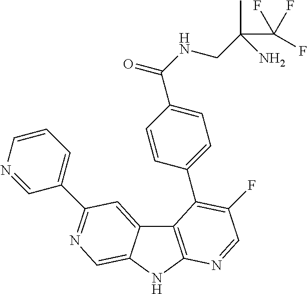 | N-(2-amino-3,3,3-trifluoro- 2-methylpropyl)-4-[3- fluoro-6-(pyridin-3-yl)-9H- pyrrolo[2,3-b:5,4-c′]di- pyridin-4-yl]benzamide | 2.62 | 509.14 |

### Examples 92 to 98

### Example 92

### 2′,5′-dichloro-5-fluoro[3,4′]bipyridinyl-2-ylamine

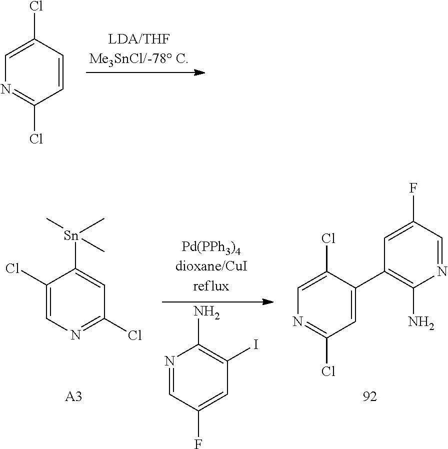

A solution of 10.45 mL (74.32 mmol) of diisopropylamine in 1000 ml of tetrahydrofuran is stirred under an argon atmosphere at −78° C. 28.6 ml (74.32 mmol) of a solution of butyllithium (2.5 M) in hexane are added slowly, while keeping the temperature below −70° C. After stirring for 30 minutes at −78° C., a solution of 10 g (67.57 mmol) of 2,5-dichloropyridine in 200 ml of tetrahydrofuran is added over 25 minutes. After stirring for 1 hour 30 minutes at −78° C., 17.5 g (87.84 mmol) of trimethyltin chloride and 100 ml of tetrahydrofuran are added dropwise. The reaction mixture is stirred for 14 hours at room temperature and then hydrolysed with 400 ml of saturated ammonium chloride solution and 350 ml of water. The suspension is extracted with three times 500 mL of ethyl acetate. The organic solution is separated out by settling, dried over magnesium sulfate and then concentrated to dryness on a rotary evaporator to give 27 g of a mobile oil, which is purified by chromatography on silica (200 g), eluent: 97.5/2.5 cyclohexane/ethyl acetate by volume. 12.46 g (59%) of 2,5-dichloro-4-trimethylstannylpyridine A3 are obtained in the form of a white powder.

The reaction is performed in four runs of about 1.5 g in a 20 mL reactor of Biotage vial type and a Biotage microwave oven.

A suspension of 1.5 g of 5-fluoro-3-iodo-2-aminopyridine (63 mmol), 2.15 g (6.93 mmol) of 2,5-dichloro-4-trimethylstannylpyridine, 0.25 g (1.32 mmol) of copper (I) chloride and 0.515 g (0.44 mmol) of tetrakis(triphenylphosphine)palladium(0) in 15 ml of dioxane and 0.1 ml of dimethylformamide is heated at 120° C. for 1 hour 20 minutes by microwave.

The four crude reaction products are then combined and diluted in 80 mL of ethyl acetate, and washed with 90 mL of saturated sodium bicarbonate solution and then with 90 ml of water. After drying over magnesium sulfate, the organic phase is concentrated on a rotary evaporator and then purified by chromatography on silica (90 g), eluent: 9/1 and then 8/2 cyclohexane/ethyl acetate by volume to give 4.22 g (68%) of 2′,5′-dichloro-5-fluoro[3,4]bipyridinyl-2-ylamine 92 in the form of a pale yellow powder.

UPLC-MS-DAD-ELSD: Tr (min)=3.35; [M+H]⁺: m/z 299.06 and 301.08

### Example 93

### 6-chloro-3-fluoro-9H-pyrrolo[2,3-b:5,4-c′]dipyridine

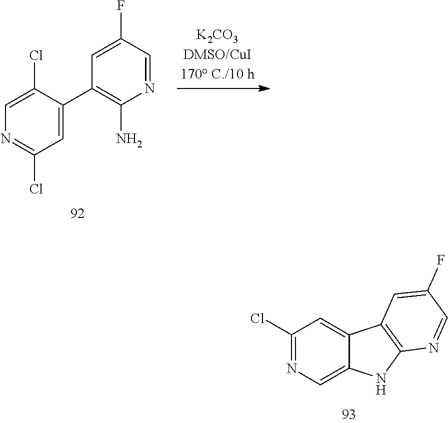

A suspension of 4.2 g (16.27 mmol) of 2′,5′-dichloro-5-fluoro[3,4′]bipyridinyl-2-ylamine, 7 g (48.81 mmol) of potassium carbonate and 0.62 g (3.25 mmol) copper iodide in 100 ml of dimethyl sulfoxide is heated on an oil bath at 170° C. for 3 hours 30 minutes. The reaction mixture is then stirred in 300 g of ice and 250 ml of 28% aqueous ammonia solution for 1 hour, and then extracted with five times 300 mL of ethyl acetate. The organic phases are combined, dried over magnesium sulfate and concentrated to dryness on a rotary evaporator to give an ochre-coloured solid, which is triturated in 80 mL of ethyl ether. The suspension is filtered, drained by suction and dried to give 1.75 g (49%) of 6-chloro-3-fluoro-9H-dipyrido[2,3-b:4′,3′-d]pyrrole 93 in the form of an ochre-coloured powder.

UPLC-MS-DAD-ELSD: Tr (min)=0.73; [M+H]⁺: m/z 257.95 and 259.93

### Example 94

### 6-chloro-3-fluoro-9-[(4-methylphenyl)sulfonyl]-9H-pyrrolo[2,3-b:5,4-c′]dipyridine

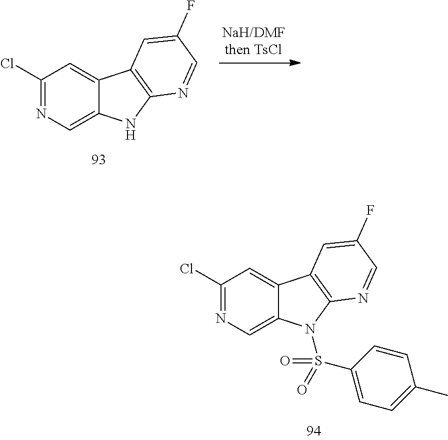

6-Chloro-3-fluoro-9H-dipyrido[2,3-b:4′,3′-d]pyrrole 93 (800 mg, 3.6 mmol) is dissolved in 20 ml of DMF in a dry one-necked flask under argon (gentle heating to 35° C. may be necessary in certain cases for complete dissolution). Sodium hydride (245 mg, 6.1 mmol, 1.7 eq.) is added in a single portion, and the reaction mixture is then stirred under an inert atmosphere for 3 hours. Tosyl chloride dissolved in 2 ml of DMF is then added (addition time of about two minutes). After one hour, the reaction medium is poured into a mixture of aqueous 10% NaHCO₃ solution (50 mL) and water (30 mL). The precipitate is filtered off and then drained by suction with 50 ml of water. After drying (overnight), the precipitate is purified by chromatography on silica gel (no need for slurrying on silica, since the product is sufficiently soluble in dichloromethane: 70 g SiO₂, CH₂Cl₂/EtOAc: 100/0 to 90/10). 1.02 g (75%) of 6-chloro-3-fluoro-9-(toluene-4-sulfonyl)dipyrido[2,3-b:4′,3′-d]pyrrole 94 are obtained.

UPLC-MS-DAD-ELSD: Tr (min)=1.10; [M+H]⁺: m/z 375.99 and 377.95

### Example 95

### 6-chloro-3-fluoro-4-iodo-9-[(4-methylphenyl)sulfonyl]-9H-pyrrolo-[2,3-b:5,4-c′]dipyridine

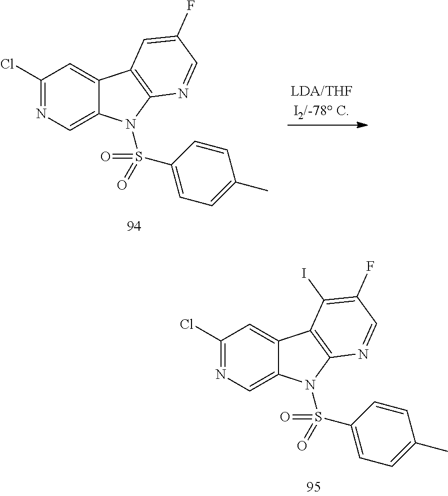

To a solution of 0.37 mL (2.6 mmol) of diisopropylamine in 6 ml of tetrahydrofuran is added under an argon atmosphere, dropwise over about two minutes at −78° C., 1 ml (2.5 mmol) of a solution of butyllithium (2.5 M) in hexane. After stirring for 30 minutes at −78° C., a solution of 94 (620 mg, 1.6 mmol) in 25 ml of THF is then added slowly (total addition time of about 10 minutes). After 3 hours at −78° C., a solution of diiodine (670 mg) in 2 ml of THF is than added rapidly. After stirring for 30 minutes at −78° C., the reaction medium is poured into a mixture of ethyl acetate (150 mL) and half-saturated aqueous ammonium chloride solution (50 ml of saturated aq. NH₄Cl+50 ml of water). The phases are separated and the organic phase is then washed with aqueous 5% sodium thiosulfate solution. The organic phase is dried over MgSO₄, filtered and evaporated under reduced pressure. 615 mg (74%) of 6-chloro-3-fluoro-4-iodo-9-(toluene-4-sulfonyl)dipyrido[2,3-b:4′,3′-d]pyrrole 95 are obtained.

UPLC-MS-DAD-ELSD: Tr (min)=1.19; [M+H]⁺: m/z 501.89 and 503.85

### Example 96

### 6-chloro-3-fluoro-4-iodo-9H-pyrrolo[2,3-b:5,4-c′]dipyridine

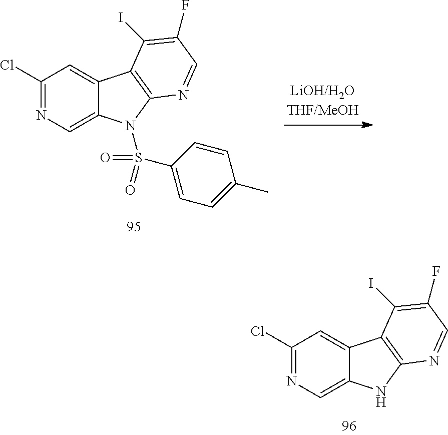

6-Chloro-3-fluoro-4-iodo-9-(toluene-4-sulfonyl)dipyrido[2,3-b:4′,3′-d]pyrrole 95 (615 mg, 1.2 mmol) is dissolved in 40 ml of THF and 15 ml of MeOH. Aqueous lithium hydroxide monohydrate solution (503 mg, 12 mmol in 20 ml of water) is then added rapidly. After stirring for 2 hours, 80 ml of water are added and the medium is then acidified by addition of aqueous 1N hydrochloric acid solution (about 12 mL) to pH 4. The precipitate is filtered off on a porosity 4 sinter funnel, drained by suction with 20 ml of water and then dried (overnight). 415 mg (97%) of 6-chloro-3-fluoro-4-iodo-9H-dipyrido[2,3-b:4′,3′-d]pyrrole 96 are obtained.

UPLC-MS-DAD-ELSD: Tr (min)=0.93; [M+H]⁺: m/z 347.91 and 349.90

### Example 97

### 4-[3-fluoro-6-chloro-9H-pyrrolo[2,3-b:5,4-c′]dipyridin-4-yl]benzoic acid

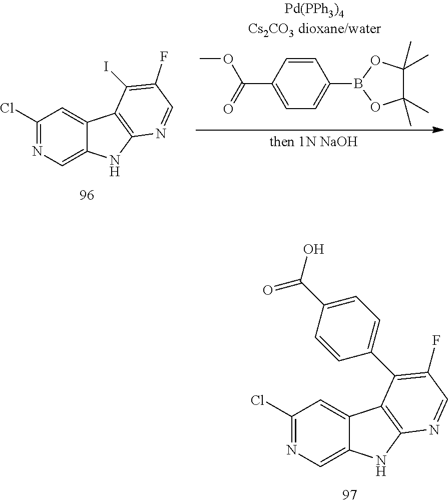

625 mg of 6-chloro-3-fluoro-4-iodo-9H-dipyrido[2,3-b:4′,3′-d]pyrrole 96, 1.415 g of 4-methoxycarbonylphenylboronic acid pinacol ester, 208 mg of tetrakis(triphenyl-phosphine)palladium(0), 1.76 g of caesium carbonate in 40 ml of 1,4-dioxane and 8 ml of water are placed in a three-necked flask, and the mixture is then refluxed for 18-20 hours. 4 ml of aqueous 1N sodium hydroxide solution are added to the reaction medium, and the resulting mixture is refluxed for a further one hour. After cooling, the reaction medium is poured into a mixture of water and ethyl acetate with vigorous stirring. The phases are separated and the pH of the aqueous phase is then brought to 4 by adding aqueous hydrochloric acid solution. The precipitate that forms during this acidification is filtered off, drained by suction and dried under vacuum. 540 mg (88%) of 4-[3-fluoro-6-chloro-9H-pyrrolo[2,3-b:5,4-c′]dipyridin-4-yl]benzoic acid 97 are thus obtained,

UPLC-MS-DAD-ELSD: Tr (min)=0.73; [M−H]⁻ m/z 340.08 and 342.02

### Example 98

### 4-[3-fluoro-6-(1-methyl-1H-pyrazol-4-yl)-9H-pyrrolo[2,3-b:5,4-c′]dipyridin-4-yl]benzoic acid

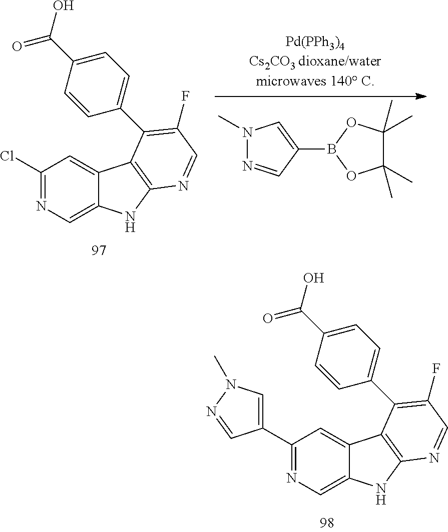

440 mg of 4-[3-fluoro-6-chloro-9H-pyrrolo[2,3-b:5,4-c′]dipyridin-4-yl]benzoic acid 97, 148 mg of tetrakis(triphenylphosphine)palladium(0), 804 mg of 1-methyl-4-(4,4,5,5-tetramethyl-1,3,2-dioxaborolan-2-yl)-1H-pyrazole, 1.26 g of caesium carbonate in 25 ml of 1,4-dioxane and 5 ml of water are placed in a 50 mL microwave reactor, and the tube is sealed and then subjected to microwave irradiation at 140° C. for 2 hours. The reaction mixture is diluted with 50 ml of ethyl acetate and washed with 50 ml of water. After separation of the phases by settling, the aqueous phase is extracted with 100 ml of ethyl acetate and the organic phases are then combined, dried over magnesium sulfate, filtered and then concentrated under vacuum. The residue is purified by chromatography on a column of silica (eluent: 100/0 to 95/5 CH₂Cl₂/MeOH), 321 mg (64%) of 4-[3-fluoro-6-(1-methyl-1H-pyrazol-4-yl)-9H-pyrrolo-[2,3-b:5,4-c′]dipyridin-4-yl]benzoic acid 98 are obtained in the form of a yellow solid.

UPLC-MS-DAD-ELSD: Tr (min)=0.55; [M+H]⁺: m/z 388.14 and [M−H]⁻: m/z 386.20

### Examples 99 to 105

### General Procedure for Examples 99 to 105

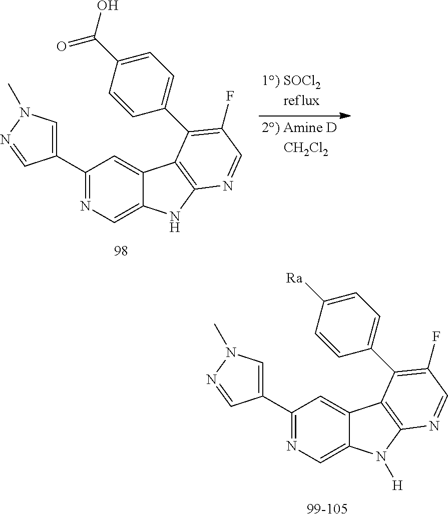

A mixture of 100 mg of 4-[3-fluoro-6-(1-methyl-1H-pyrazol-4-yl)-9H-pyrrolo[2,3-b:5,4-c′]dipyridin-4-yl]benzoic acid and 12 ml of thionyl chloride is refluxed for 2 hours. The reaction mixture is concentrated to dryness under reduced pressure to give 105 mg of 4-[3-fluoro-6-(1-methyl-1H-pyrazol-4-yl)-9H-pyrrolo[2,3-b:5,4-c′]dipyridin-4-yl]-benzoyl chloride in the form of a yellow powder.

The 4-[3-fluoro-6-(1-methy)-1H-pyrazol-4-yl)-9H-pyrrolo[2,3-b:5,4-c′]dipyridin-4-yl]-benzoyl chloride (1 eq.) is suspended in dichloromethane (50 mL per 1 mmol of acyl chloride). 5 to 10 equivalents of the amine O are dissolved in dichloromethane (same volume used as for placing 18 in suspension) in a dry round-bottomed flask under an argon atmosphere. The suspension is then added to the solution containing D with stirring at room temperature. The mixture is left stirring for between 2 and 20 hours. Methanol is then added until the precipitate disappears, and silica (10 mg per 1 mg of 98 used in the reaction) is then added. The solvents are evaporated off under reduced pressure and the product is recovered by chromatography on silica gel (eluent: CH₂Cl₂/MeOH or CH₂Cl₂/2N NH₃ in MeOH: gradient from 100/0 to 85/15).

Examples 99 to 105 obtained via this method are described in Table 6.

TABLE 6

| | | | Retention | [M + H⁺] |
|---|---|---|---|---|
| Ex. No. | Structure | Name | time (min) | observed |
| 99 | 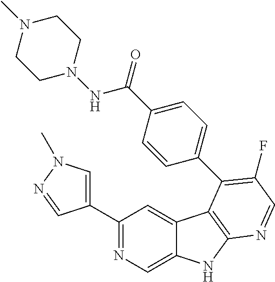 | 4-[3-fluoro-6-(1-methyl-1H- pyrazol-4-yl)-9H-pyrrolo- [2,3-b:5,4-c′]dipyridin-4-yl]- N-(4-methylpiperazin-1- yl)benzamide | 0.37 | 485.14 |
| 100 | 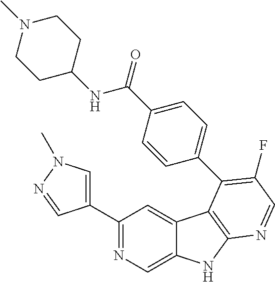 | 4-[3-fluoro-6-(1-methyl-1H- pyrazol-4-yl)-9H-pyrrolo- [2,3-b:5,4-c′]dipyridin-4-yl]- N-(4-methylpiperidin-4- yl)benzamide | 0.40 | 484.16 |
| 101 | 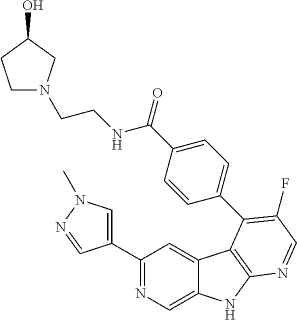 | 4-[3-fluoro-6-(1-methyl-1H- pyrazol-4-yl)-9H-pyrrolo- [2,3-b:5,4-c′]dipyridin-4-yl]- N-{2-[(3R)-3-hydroxypyr- rolidin-1-yl]ethyl} benzamide | 0.38 | 500.38 |
| 102 | 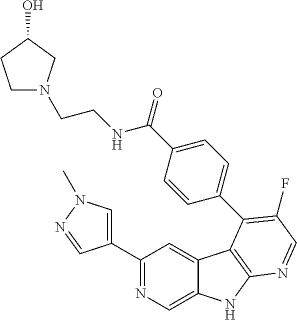 | 4-[3-fluoro-6-(1-methyl-1H- pyrazol-4-yl)-9H-pyrrolo- [2,3-b:5,4-c′]dipyridin-4-yl]- N-{2-[(3S)-3-hydroxypyr- rolidin-1-yl]ethyl} benzamide | 0.39 | 500.38 |
| 103 | 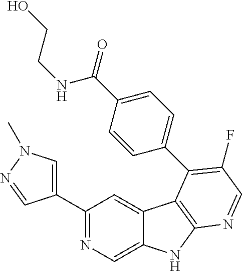 | 4-[3-fluoro-6-(1-methyl-1H- pyrazol-4-yl)-9H-pyrrolo- [2,3-b:5,4-c′]dipyridin-4-yl]- N-(2-hydroxyethyl) benzamide | 0.45 | 431.22 |
| 104 | 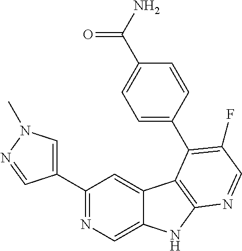 | 4-[3-fluoro-6-(1-methyl-1H- pyrazol-4-yl)-9H-pyrrolo- [2,3-b:5,4-c′]dipyridin-4-yl]- benzamide | 0.46 | 387.16 |
| 105 | 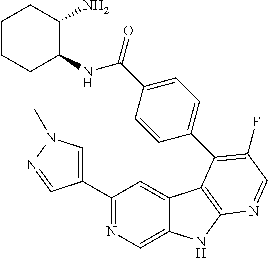 | N-[(1S,2S)-2- aminocyclohexyl]-4-[3- fluoro-6-(1-methyl-1H- pyrazol-4-yl)-9H-pyrrolo- [2,3-b:5,4-c′]dipyridin-4-yl]- benzamide | 0.47 | 484.38 |

### Examples 106 and 107

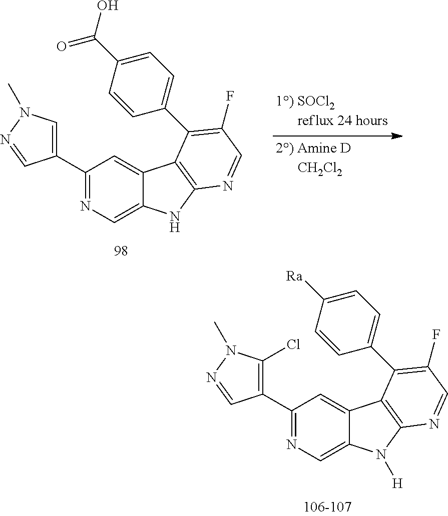

A mixture of 100 mg of 4-[3-fluoro-6-(1-methyl-1H-pyrazol-4-yl)-9H-pyrrolo[2,3-b:5,4-c′]dipyridin-4-yl]benzoic acid and 20 ml of thionyl chloride is refluxed for 24 hours. The reaction mixture is concentrated to dryness under reduced pressure to give 105 mg of 4-[3-fluoro-6-(5-chloro-1-methyl-1H-pyrazol-4-yl)-9H-pyrrolo[2,3-b:5,4-c′]dipyridin-4-yl]benzoyl chloride in the form of a yellow powder.

The 4-[3-fluoro-6-(5-chloro-1-methyl-1H-pyrazol-4-yl)-9H-pyrrolo[2,3-b:5,4-c′]dipyridin-4-yl]benzoyl chloride (1 eq.) is suspended in dichloromethane (50 mL per 1 mmol of acyl chloride). 5 to 10 equivalents of the amine are dissolved in dichloromethane (same volume used as for placing 18 in suspension) in a dry round-bottomed flask under an argon atmosphere. The suspension is then added to the solution containing D with stirring at room temperature. The mixture is stirred for 2 hours. Methanol is then added until the precipitate disappears, and silica (10 mg per 1 mg of 98 used in the reaction) is then added. The solvents are evaporated off under reduced pressure and the product is recovered by chromatography on silica gel (eluent: CH₂Cl₂/MeOH or CH₂Cl₂/2N NH₃ in MeOH: gradient from 100/0 to 85/15).

Examples 106 and 107 obtained via this method are described in Table 7.

TABLE 7

| | | | Retention | [M + H⁺] |
|---|---|---|---|---|
| Ex. No. | Structure | Name | time (min) | observed |
| 106 | 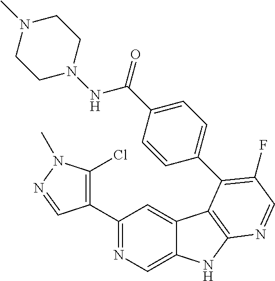 | 4-[6-(5-chloro-1-methyl-1H- pyrazol-4-yl)-3-fluoro-9H- pyrrolo[2,3-b:5,4-c′]dipyridin-4- yl]-N-(4-methylpiperazin-1- yl)benzamide | 2.34 | 519.16 and 521.13 |
| 107 | 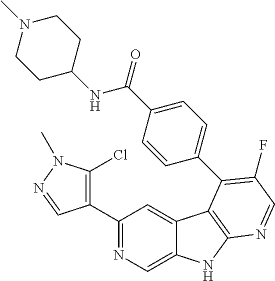 | 4-[6-(5-chloro-1-methyl-1H- pyrazol-4-yl)-3-fluoro-9H- pyrrolo[2,3-b:5,4-c′]dipyridin-4- yl]-N-(1-methylpiperidin-4- yl)benzamide | 2.45 | 518.17 |

### Examples 108 and 109

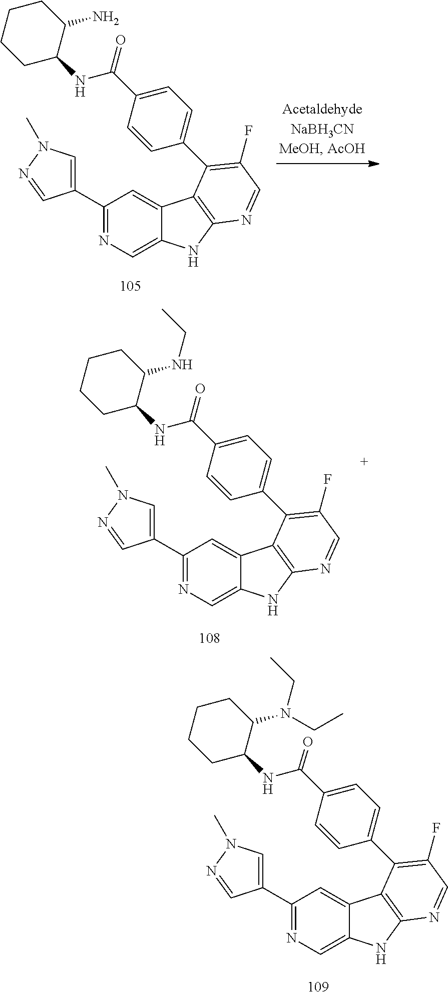

13 mg of N-[(1S,2S)-2-aminocyclohexyl]-4-[3-fluoro-6-(1-methyl-1H-pyrazol-4-yl)-9H-pyrrolo[2,3-b:5,4-c′]dipyridin-4-yl]benzamide 105 are dissolved in 2.5 ml of methanol and 0.1 mL of glacial acetic acid in a dry tube under an argon atmosphere. Acetaldehyde is then introduced via a syringe (35 μL of a freshly prepared 1M methanolic solution of acetaldehyde). The mixture is stirred for 5 minutes and sodium cyanoborohydride is then added in a single portion. After 1 hour 30 minutes, the mono-reaction product 108 is predominant; when the reaction is left stirring overnight, the diethylamino derivative 109 is then predominant. In both cases, the reaction is hydrolysed with aqueous 5% sodium bicarbonate solution and then extracted with 40 mL of ethyl acetate. After separating the phases, the organic phase is dried over MgSO₄, filtered and evaporated. The residue is purified by chromatography on silica gel (5 g of silica, eluent: CH₂Cl₂/2N NH₃ in MeOH: 100/0 to 98/2). In each case, 10 mg of compounds 108 and 109 are obtained.

Examples 108 and 109 obtained via this method are described in Table 8.

TABLE 8

| | | | Retention | [M + H⁺] |
|---|---|---|---|---|
| Ex. No. | Structure | Name | time (min) | observed |
| 108 | 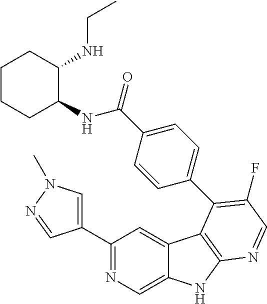 | N-[(1S,2S)-2-(ethylamino)- cyclohexyl]-4-[3-fluoro-6-(1- methyl-1H-pyrazol-4-yl)-9H- pyrrolo[2,3-b:5,4-c′]dipyridin-4- yl]benzamide | 2.52 | 512.35 |
| 109 | 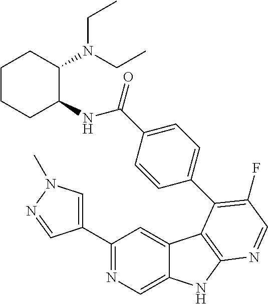 | N-[(1S,2S)-2-(diethylamino)- cyclohexyl]-4-[3-fluoro-6-(1- methyl-1H-pyrazol-4-yl)-9H- pyrrolo[2,3-b:5,4-c′]dipyridin-4- yl]benzamide | 2.62 | 540.37 |

### Examples 110 to 113

### Examples 110 and 111

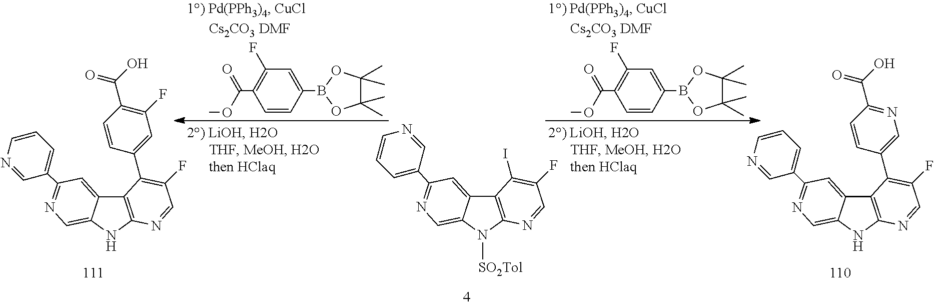

250 mg of 3-fluoro-4-iodo-9-[(4-methylphenyl)sulfonyl]-6-(pyridin-3-yl)-9H-pyrrolo-[2,3-b:5,4-c′]dipyridine 4, 3 equivalents of the appropriate pinacol boronate, 53 mg of tetrakis(triphenylphosphine)palladium(0), 300 mg of caesium carbonate and 68 mg of copper (I) chloride are suspended in 4.5 ml of DMF in a 5 mL microwave tube, and the tube is sealed and subjected to microwave irradiation at 120° C. for 1 hour. The reaction medium is hydrolysed with 100 ml of water and the resulting mixture is then extracted with twice 250 ml of ethyl acetate. The organic phases are combined, dried over sodium sulfate, filtered and concentrated to dryness under reduced pressure. The residue is then taken up in a mixture of tetrahydrofuran (2.5 mL) and methanol (2.5 mL), and 2.5 mL of aqueous 2N lithium hydroxide solution are then added. After total disappearance of the starting material, water is added to the reaction medium and the pH is brought to 4 by adding aqueous hydrochloric acid solution. The precipitate formed is isolated by filtration, rinsed with distilled water, drained by suction and then dried under vacuum.

138 mg (74%) of 110 are thus obtained.

UPLC-MS-DAD-ELSD: Tr (min)=0.57; [M+H]⁺: m/z 386.12

144 mg (69%) of 111 are thus obtained.

UPLC-MS-DAD-ELSD: Tr (min)=0.69; [M+H]⁺: m/z 403.11

### Examples 112 and 113

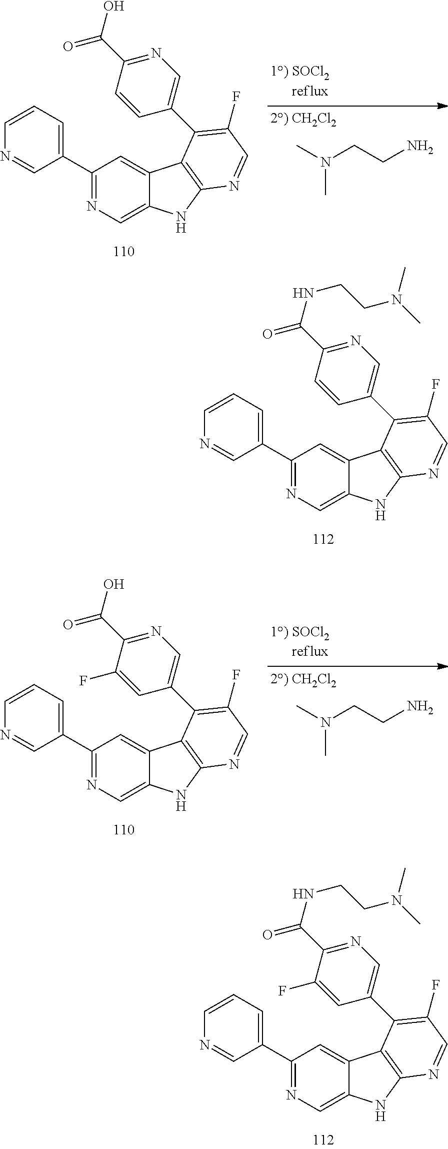

0.35 mmol of the appropriate acid (110 or 111) is suspended in 10 ml of thionyl chloride, and the mixture is then refluxed overnight. The reaction mixture is concentrated to dryness under reduced pressure. The residue is suspended in 5 ml of dichloromethane, and 3.5 mmol (10 eq.) of N,N-dimethylethylenediamine are than added. After stirring overnight at room temperature, methanol is added until the precipitate disappears, and silica (1 to 2 g) is then added. The solvents are evaporated off under reduced pressure and the product is recovered by chromatography on silica gel (eluent: CH₂Cl₂/2N NH₃ in MeOH: gradient from 100/0 to 90/10).

Examples 112 and 113 obtained via this method are described in Table 9.

TABLE 9

| | | | Retention | [M + H⁺] |
|---|---|---|---|---|
| Ex. No. | Structure | Name | time (min) | observed |
| 112 | 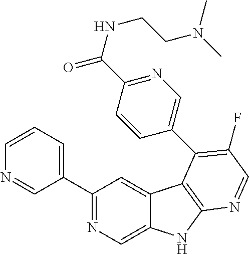 | N-[2-(dimethylamino)ethyl]-5- [3-fluoro-6-(pyridin-3-yl)-9H- pyrrolo[2,3-b:5,4-c′]dipyridin-4- yl]pyridine-2-carboxamide | 0.38 | 456.16 |
| 113 | 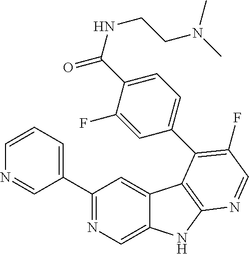 | N-[2-(dimethylamino)ethyl]-2- fluoro-4-[3-fluoro-6-(pyridin-3- yl)-9H-pyrrolo[2,3-b:5,4-c′]di- pyridin-4-yl]benzamide | 0.41 | 473.17 |

### Examples 114 to 120

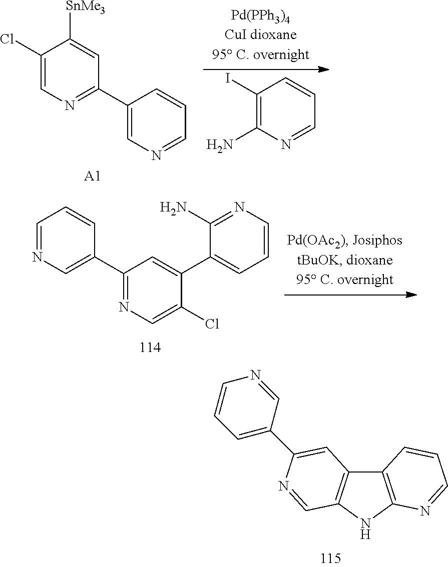

2.90 g (13.8 mmol) of 2-amino-3-iodopyridine and 4.7 g (13.2 mmol) of 5-chloro-4-(trimethylstannyl)-2,3′-bipyridine A1 are placed in 250 three-necked flask, and 1.07 g (7% mol) of tetrakis(triphenylphosphine)palladium(0) and 531 mg copper iodide(I) are added in a single portion, along with 80 ml of 1,4-dioxane. The mixture is refluxed for 18 hours. After cooling, the reaction medium is treated with aqueous 10% sodium hydrogen carbonate solution and then diluted with ethyl acetate. After separation of the phases by settling, the aqueous phase is extracted twice with ethyl acetate. The combined organic phases are dried over anhydrous sodium sulfate, filtered and concentrated under reduced pressure. The residue is taken up in a mixture of dichloromethane and methanol, and is then filtered by suction to give 1.9 g (51%) of 5′-chloro-3,2′:4′,3″-terpyridin-2″-amine 114 in the form of a pale beige-coloured solid, which is used in the following step without further purification. 237 mg of (R)-(−)-1-[(S)-2-(dicyclohexylphosphino)ferrocenyl]ethyldi-tert-butylphosphine and 85 mg of palladium (II) acetate are dissolved in 4 ml of anhydrous 1,4-dioxane in a dry 25 ml round-bottomed flask under an argon atmosphere, and the mixture is stirred for 10 minutes at 40° C.

1.8 g of 5′-chloro-3,2′:4′,3″-terpyridin-2″-amine are dissolved in 30 ml of anhydrous 1,4-dioxane with 1.12 g of potassium tert-butoxide in a 100 ml reactor under argon, and the catalyst solution prepared previously is then added. The reaction mixture is refluxed for 18 hours. After concentrating under reduced pressure, the product is purified by chromatography on a column of silica (eluent: 98/2 to 92/8 CH₂Cl₂/MeOH) to give 1.06 g (68%) of 6-(pyridin-3-yl)-9H-pyrrolo[2,3-b:5,4-c′]dipyridine in the form of a light brown solid.

1H NMR (400 MHz, DMSO-d6) δ ppm: 7.35 (dd, J=8.0, 4.5 Hz, 1H) 7.53 (dd, J=8.5, 4.5 Hz, 1H) 8.51 (dt, J=8.5, 1.5 Hz, 1H) 8.59 (dd, J=4.5, 1.5 Hz, 1H) 8.62 (dd, J=4.5, 1.5 Hz, 1H) 8.72 (dd, J=8.0, 1.5 Hz, 1H) 8.91 (d, J=1.0 Hz, 1H) 9.03 (d, J=1.0 Hz, 1H) 9.37 (d, J=1.5 Hz, 1H) 12.3 (broad m, 1H)

LC-MS-DAD-ELSD: [M+H]⁺: m/z 247

### Example 116

### 3-bromo-6-(pyridin-3-yl)-9H-pyrrolo[2,3-b:5,4-c′]dipyridine

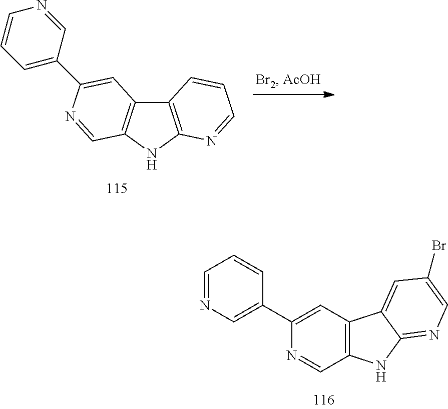

360 mg of 115, 15 mL of acetic acid and 10 ml of dimethylformamide are placed in a round-bottomed flask. After stirring, 0.3 ml of bromine is added dropwise. After stirring for 3 hours at room temperature, the precipitate is filtered off and then rinsed with aqueous sodium thiosulfate solution and water. After drying, 463 mg (97%) of 3-bromo-6-(pyridin-3-yl)-9H-pyrrolo[2,3-b:5,4-c′]dipyridine 116 are obtained.

1H NMR (400 MHz, DMSO-d6) δ ppm: 7.54 (dd, J=8.0, 4.9 Hz, 1H) 8.47 (dt, J=8.0, 2.0 Hz, 1H) 8.60 (dd, J=4.9, 2.0 Hz, 1H) 8.69 (d, J=2.4 Hz, 1H) 8.93 (s, 1H) 9.00 (d, J=2.4 Hz, 1H) 9.05 (s, 1H) 9.34 (d, J=2.0 Hz, 1H) 1255 (broad m, 1H)

### Example 117

### 3-(2-methoxyethoxy)-6-(pyridin-3-yl)-9H-pyrrolo[2,3-b:5,4-c′]dipyridine

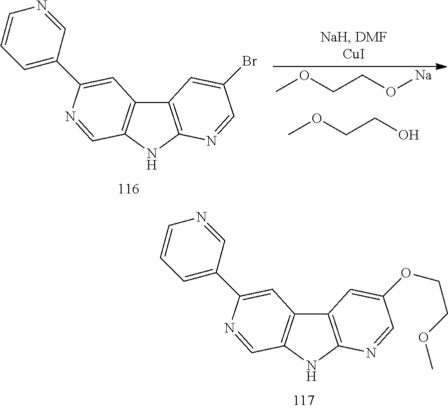

180 mg of 3-bromo-6-(pyridin-3-yl)-9H-pyrrolo[2,3-b:5,4-c′]dipyridine 116 and 169 mg copper(I) iodide are dissolved in 4.1 ml of a 21% solution of sodium methoxyethoxide in methoxyethanol and 0.4 ml of dimethylformamide in a 5 mL microwave tube, and the tube is sealed and then subjected to microwave irradiation for 45 minutes at 120° C. After cooling, the reaction medium is poured into a mixture of 50 ml of ethyl acetate and aqueous ammonium chloride solution with vigorous stirring. After separation of the phases by settling, the organic phase is dried over sodium sulfate, filtered and concentrated to dryness. The residue is purified by chromatography on a column of silica (eluent: 100/0 to 95/5 CH₂Cl₂/MeOH) to give 132 mg (74%) of 3-(2-methoxyethoxy)-6-(pyridin-3-yl)-9H-pyrrolo[2,3-b:5,4-C′]dipyridine 117.

1H NMR (400 MHz, DMSO-d6) δ ppm 3.36 (s, 3H) 3.76 (t, J=4.9 Hz, 2H) 4.27 (t, J=4.4 Hz, 2H) 7.53 (dd, J=7.9, 4.8 Hz, 1H) 8.39-8.41 (m, 2H) 8.48 (dt, J=8.0, 2.0 Hz, 1H) 8.58 (dd, J=4.6, 1.7 Hz, 1H) 8.87 (d, J=1.0 Hz, 1H) 8.99 (d, J=1.0 Hz, 1H) 9.34 (d, J=2.2 Hz, 1H) 12.11 (br. s., 1H).

LC-MS-DAD-ELSD: Tr (min)=2.31; [M+H]⁺: m/z 321; [M−H]⁻: m/z 319.

### Example 118

### 3-(2-methoxyethoxy)-9-[(4-methylphenyl)sulfonyl]-6-(pyridin-3-yl)-9H-pyrrolo[2,3-b:5,4-e]dipyridine

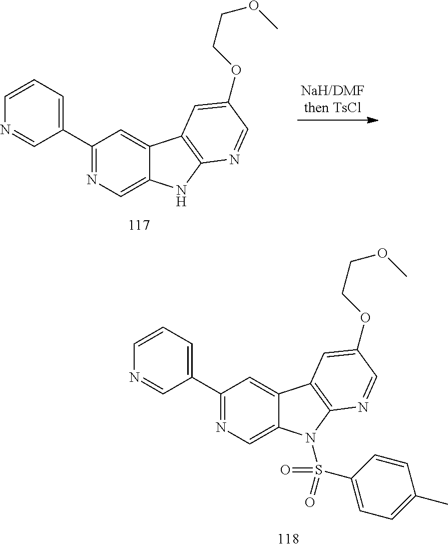

117 (70 mg, 0.22 mmol) is dissolved in 2 ml of DMF in a dry one-necked flask under argon. Sodium hydride (15 mg, 1.7 eq.) is added in a single portion, and the reaction mixture is then stirred under an inert atmosphere for 3 hours. Tosyl chloride dissolved in 0.5 ml of DMF is then added (addition time of about two minutes). After one hour, the reaction medium is poured into a mixture of aqueous 10% NaHCO₃ solution (50 mL) and water (50 mL) and extracted with twice 100 mL of ethyl acetate. After drying the combined organic phases over MgSO₄, they are filtered and then evaporated under reduced pressure. The residue is purified by chromatography on silica gel (25 g of SiO₂, 100/0 to 95/5 CH₂Cl₂/MeOH). 77 mg (72%) of 6-chloro-3-(2-methoxyethoxy)-9-(toluene-4-sulfonyl)dipyrido[2,3-b:4′,3′-d]pyrrole 118 are obtained.

UPLC-MS-DAD-ELSD: Tr (min)=1.18; [M+H]⁺: m/z 475.33

### Example 119

### 4-iodo-3-(2-methoxyethoxy)-6-(pyridin-3-yl)-9H-pyrrolo[2,3-b:5,4-c′]dipyridine

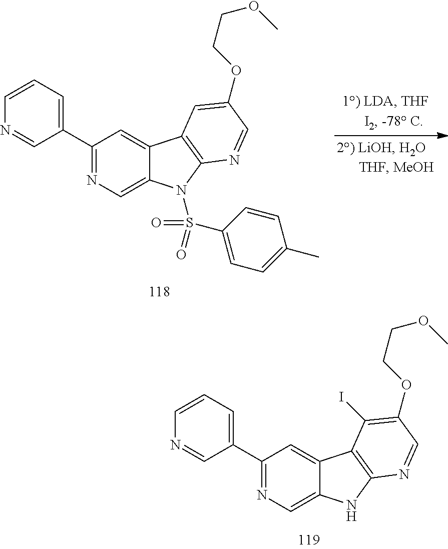

A solution of 34 μL (0.24 mmol) of diisopropylamine in 1 ml of tetrahydrofuran is stirred under an argon atmosphere at −78° C. 0.1 ml (2.4 mmol) of a solution of butyllithium (2.4 M) in hexane is added slowly dropwise over about two minutes. After stirring for 30 minutes at −78° C., a solution of 118 (77 mg, 0.16 mmol) in 3 ml of THF is then added slowly (total addition time of about 10 minutes). After 3 hours at −78° C., a solution of diiodine (66 mg) in 0.5 ml of THF is added rapidly. After stirring for 30 minutes at −78° C., the reaction medium is poured into a mixture of ethyl acetate (50 mL) and half-saturated aqueous ammonium chloride solution (30 ml of saturated aqueous NH₄Cl+30 mL of water). The phases are separated and the organic phase is then washed with aqueous 5% sodium thiosulfate solution. The organic phase is dried over MgSO₄, filtered and evaporated under reduced pressure. 89 mg (9196) of 6-chloro-3-(2-methoxyethoxy)-4-iodo-9-(toluene-4-sulfonyl)dipyrido[2,3-b:4′,3′-d]pyrrole are obtained. The product is used in the following step without further purification.

6-Chloro-3-(2-methoxyethoxy)-4-iodo-9-(toluene-4-sulfonyl)dipyrido[2,3-b:4′,3′-d]pyrrole (85 mg, 0.14 mmol) is dissolved in 1.5 ml of THF and 1.5 ml of MeOH. Aqueous lithium hydroxide monohydrate solution (61 mg, 2.55 mmol in 1.5 ml of water) is then added rapidly. After stirring for 2 hours, 10 ml of water are added and the medium is then acidified by addition of aqueous hydrochloric acid solution to pH 4. The precipitate is filtered off on a porosity 4 sinter funnel, drained by suction with 5 ml of water and then dried overnight under vacuum. 40 mg (97%) of 6-chloro-3-(2-methoxyethoxy)-4-iodo-9H-dipyrido[2,3-b:4′,3′-d]pyrrole 119 are obtained.

UPLC-MS-DAD-ELSD: Tr (min)=0.85; [M+H]⁺: m/z 447.00

### Example 120

### N-[2-(dimethylamino)ethyl]-4-[3-(2-methoxyethoxy)-6-(pyridin-3-yl)-9H-pyrrolo[2,3-b:5,4-c′]dipyridin-4-yl]benzamide

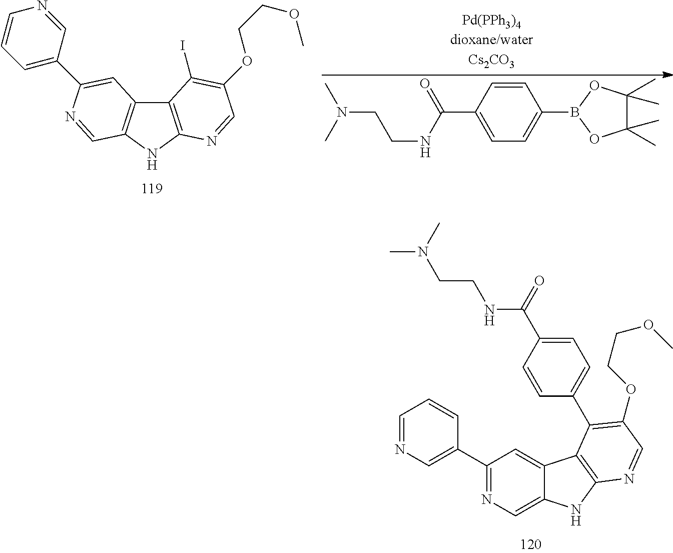

40 mg of 119, 57 mg N-(2-dimethylaminoethyl)-4-(4,4,5,5-tetramethyl-1,3,2-dioxaborolan-2-yl)benzamide, 10 mg of tetrakis(triphenylphosphine)palladium(0) and 58 mg of caesium carbonate are suspended in 0.5 ml of 1,4-dioxane and 0.1 ml of water in a 2 mL microwave reactor, and the tube is sealed and then subjected to microwave irradiation for 1 hour at 130° C. The reaction mixture is filtered and then poured into 25 ml of water and 50 ml of ethyl acetate with vigorous stirring. After separation of the phases by settling, the aqueous phase is extracted with 30 ml of ethyl acetate and the combined organic phases are dried over magnesium sulfate, filtered and then evaporated under reduced pressure. The residue is purified by chromatography on a column of silica (eluent: 100/0 to 90/10 CH₂Cl₂/2N NH₃ in MeOH) to give 30 mg of N-[2-(dimethylamino)ethyl]-4-[3-(2-methoxyethoxy)-6-(pyridin-3-yl)-9H-pyrrolo[2,3-b:5,4-c′]dipyridin-4-yl]benzamide.

UPLC-MS-DAD-ELSD: Tr (min)=0.39; [M+H]⁺: m/z 511.25

### Example 121

### Pharmaceutical Composition

Tablets corresponding to the following formula were prepared:

| | |
|---|---|
| Product of Example 44 | 0.2 g |
| Excipient for a finished tablet weighing | 1 g |
| (details of the excipient: lactose, talc, starch, | |
| magnesium stearate). | |

### Example 122

### Pharmaceutical Composition

Tablets corresponding to the following formula were prepared:

| | |
|---|---|
| Product of Example 87 | 0.2 g |
| Excipient for a finished tablet weighing | 1 g |
| (details of the excipient: lactose, talc, starch, | |
| magnesium stearate). | |

Examples 44 and 87 are taken as examples of pharmaceutical preparation, this preparation possibly being performed, if desired, with other products illustrated in the present patent application.

### In Vitro Biochemical Test Procedures

The pharmacological properties of the compounds of the invention may be confirmed by a certain number of pharmacological assays. The pharmacological assay examples that follow were performed with compounds according to the invention.

### Example 1

### TR-FRET Assay

In order to determine the inhibition of Pim kinase activity, the compounds of the invention are tested in accordance with a routinely-used in-vitro TR-FRET assay (“Time Resolved-Fluorescence Resonance Energy Transfer”). The TR-FRET assay is based on the detection of phosphorylation of the specific residue Ser112 in the Bad protein, which was found to be a natural substrate of the Pim kinases in cells. For the assay, the following reagents are used:

Pim kinase—full-length human recombinant His6-labelled Pim-1, Pim-2 or Pim-3 protein (prepared according to J. Mol. Biol. (2005) 348, 183-193);
Bad—full-length human recombinant His6-labelled Bad protein (prepared according to J. Mol. Biol. (2005) 348, 183-193);
α-His6-APC—mouse monoclonal antibody conjugated to allophycocyanine SureLight™ directed against the His6 label (Perkin-Elmer, No. AD0059H, Waltham, Mass., United States);
α-P˜Bad-Eu—mouse monoclonal antibody (Cell Signaling Technology #9296B, Danvers, Mass., United States) directed against phosphoBad (Ser112) (7E11) labelled on request by Perkin-Elmer with the reagent LANCE™ Eu-W1024.

The assay is based on the LANCE™ technology from Perkin-Elmer: the Eu-labelled antibody binds to phospho-Ser112 and generates a TR-FRET signal by interaction with the ARC-labelled antibody directed against His6, bound to the His6 label of Bad. The TR-FRET signal is detected using a SpectraMax M5 plate reader (Molecular Devices) with the following settings: λex=340 nm, λem1=615 nm, λem2=665 nm. The ratio of the fluorescence signal at 665 nm to the fluorescence signal at 615 nm is used as the signal reading for the IC₅₀ (calculations based on the 4-parameter logistic model). The assay is performed in 384-well format; the liquid manipulations are performed using a Beckmann 3000 liquid manipulation station. The test compounds are tested at 10 concentration points in duplicate; the highest concentration of compound is typically equal to 30 μM. The ATP concentration is equal to 40 μM.

### Example 2

### Cell Viability Assay

The representative compounds of the invention are also screened as regards their effects on cell proliferation and viability, using a variety of tumoral cell lines of human source, representative of different pathological indications. These cell lines include:

Models of Haematological Cancers:
TF-1 (acute myelogenic leukaemia; AML M6 at the time of diagnosis);
K G-1 (AML; erythroleukaemia evolving into AML);
KG-1a (AML; subclone derived from immature KG-1);
EOL-1 (AML: eosinophilic leukaemia);
PL-21 (AML; M3);
ML-2 (AML; T-NHL evolving into T-ALL evolving into AML M4);
HL-60 (AML, M3);
Kasumi-1 (AML);
GDM-1 (AML);
K-562 (CML—chronic myelogenic leukaemia; blastic crisis);
JURL-MK1 (CML; blastic crisis);
DND-41 (T-ALL—T-cell acute lymphoblastic leukaemia);
Jurkat (T-ALL); NALM-6 (B-ALL B-cell ALL);
CEM (ALL; lymphosarcoma evolving into ALL);
Jeko-1 (B-NHL—B-cell non-Hodgkin lymphoma; lymphoma with coat cells derived from a large-cell variant in leukaemic transformation);
WSU-DLCL2 (B-NHL; diffuse lymphoma with large B cells);
RL (B-NHL; undifferentiated diffuse);
OCl-Ly10 (B-NHL);
DoHH-2 (B-NHL);
RPMI-8226 (MM—multiple myelomas);
JVM-2 (B-CLL—B-cell chronic lymphocytic leukaemia); and
JVM-3 (B-CLL).

Models of Solid Tumours:
HCT-116 (bowel cancer);
HT-29 (bowel cancer);
HC-15 (bowel cancer);
H460 (lung cancer; non-small-cell lung cancer);
A375 (melanoma);
B16F10 (melanoma);
MDA-A1 (breast cancer);
MDA-MB231 (breast cancer);
MDA-MB231adr (breast cancer);
PANC-1 (pancreatic cancer); and
PC-3 (prostate cancer).

In order to measure the viability, the tumour cells are incubated in 96-well or 384-well format for 48, 72 or 96 hours, preferably 72 hours, with a compound of the invention at dilutions of a factor 3, with, in general, nine doses in total, the highest dose being equal to 10 μM or 30 μM. The cell viability is evaluated by adding CellTiter-Blue® (Promega, Madison, Wis., United States) for 4 hours and endpoint readings are taken using a SpectraMax Genmini EM plate reader (Molecular Devices, Sunnyvale, Calif., United States). The CellTiter-Blue® cell viability assay measures the ability of the cells in culture to reduce resazurin to resorufin, the intensity of the fluorescence signal being directly proportional to the number of live cells. The EC₅₀ represents the concentration of compound that leads to a 50% reduction of the viability/proliferative expansion of the cells.

### Example 3

The activity of the molecules is evaluated on the JEKO or DND-41 cell line by measuring the level of phosphorylation of Bad and the total Bad content, via the ELISA technique.

The JEKO or DND-41 cells are resuspended at a concentration of 500 000 cells per ml. The cells are then diluted in RPMI-1640 medium containing 20% foetal calf serum. 225 μl of cells are placed in a plate. Serial dilutions of the molecules are then performed (8 points, three-fold dilution, range started at 10 mM). Each dilution point is then diluted to 1/100 in medium. Next, 25 μl of each of the concentrations are added to the cells and then incubated for 3 hours at 37° C.

100 μl of the cells are transferred into a plate treated with poly-D-Lysine. The plate is then incubated for 5-10 minutes at 37° C. and then centrifuged for 5 minutes at 1500 rpm. A volume of 100 μl of 8% fixing solution (solution of formaldehyde in PBS buffer) is added to each of the wells. After covering with a self-adhesive film and a lid, the plate is incubated for 20 minutes at room temperature and then stored at 4° C. overnight.

Next, the liquid is removed and two successive washes are performed with washing buffer. 100 μl of quenching buffer are added, followed by incubating for 15 minutes at room temperature. After washing, 100 μl of quenching buffer are added, followed by incubation for 1 hour at room temperature. After washing, 50 μl of primary antibody diluted to 1/250 for pBAD (Cell Signaling Cat#5284) and to 1/500 for Bad (MBL Cat#591) are added to each of the plates. Each plate is incubated for 2 hours at room temperature. After two washes, 50 μl of secondary antibody (diluted to 1/16) are added to the pBAD plate (tebu-bio ref; FE-021) and 50 μl of the HRP-conjugated IgG secondary antibody (Santa Cruz Cat #SC-2004; diluted to 1/1000) are added to the Bad plate. Incubation for 1 hour at room temperature is performed. Four washes with the washing solution are performed, followed by three washes with PBS. Finally, 100 μl of the developing solution are added, followed by incubation for 10 minutes. Finally, 100 μl of the quenching solution are added, before reading at 450 nm.

### Biochemical Results

| Example | IC50 | IC50 | IC50 | IC50 | IC50 |
|---|---|---|---|---|---|
| No. | Pim1 | Pim2 | Pim3 | CDK1 | PLK1 |
| 7 | A | B | A | C | B |
| 9 | A | A | A | B | B |
| 10 | A | B | A | C | C |
| 11 | A | C | B | | Top of |
| | | | | | pharmacopoeia |
| | | | | | Bottom of |
| | | | | | pharmacopoeia |
| 12 | A | C | A | C | C |
| 13 | A | C | B | | |
| 14 | A | C | B | | Top of |
| | | | | | pharmacopoeia |
| | | | | | Bottom of |
| | | | | | pharmacopoeia |
| 15 | B | D | C | | |
| 16 | A | C | B | | |
| 17 | A | C | B | | |
| 19 | A | B | A | C | B |
| 20 | A | B | A | B | B |
| 21 | A | C | A | B | C |
| 22 | A | B | A | C | C |
| 23 | A | A | A | C | B |
| 24 | A | C | A | C | C |
| 25 | A | C | A | C | C |
| 26 | A | A | A | C | B |
| 27 | A | A | A | C | B |
| 28 | A | A | A | B | B |
| 37 | A | A | A | B | C |
| 38 | A | A | A | B | C |
| 44 | A | A | A | B | B |
| 45 | B | D | B | B | A |
| 46 | A | C | A | B | B |
| 47 | A | A | A | B | B |
| 48 | A | C | B | B | A |
| 49 | B | D | B | C | B |
| 50 | B | D | B | B | A |
| 51 | A | B | A | C | B |
| 52 | A | B | A | C | B |
| 53 | A | B | A | C | B |
| 54 | A | B | A | B | B |
| 55 | A | B | A | B | B |
| 56 | A | A | A | B | B |
| 57 | A | C | A | B | B |
| 58 | A | B | A | C | B |
| 59 | A | B | A | B | C |
| 60 | A | B | A | B | B |
| 61 | A | B | A | C | C |
| 62 | A | C | A | B | B |
| 63 | A | B | A | C | B |
| 64 | A | B | A | C | B |
| 65 | A | B | A | C | B |
| 66 | A | B | A | C | B |
| 67 | A | A | A | C | B |
| 68 | A | A | A | C | C |
| 69 | A | B | A | B | B |
| 70 | A | C | B | C | C |
| 71 | A | C | A | C | C |
| 72 | B | D | B | C | B |
| 73 | A | B | A | B | A |
| 74 | A | B | A | C | B |
| 75 | A | B | A | B | C |
| 76 | A | B | A | B | A |
| 77 | A | C | A | C | C |
| 78 | A | B | A | B | A |
| 79 | A | B | A | C | B |
| 80 | A | B | A | B | B |
| 81 | A | C | A | C | B |
| 82 | A | B | A | B | B |
| 83 | A | A | A | | |
| 84 | A | A | A | B | B |
| 85 | A | A | A | | |
| 86 | A | A | A | | |
| 87 | A | A | A | B | B |
| 88 | A | A | A | B | B |
| 89 | A | A | A | B | B |
| 90 | A | B | A | B | B |
| 91 | B | C | B | B | B |
| 99 | A | B | A | C | D |
| 100 | A | A | A | C | C |
| 104 | A | B | A | B | B |
| 106 | A | A | A | B | C |
| 107 | A | A | A | C | D |
| 112 | B | C | B | | |
| 113 | A | B | A | | Top of |
| | | | | | pharmacopoeia |
| | | | | | Bottom of |
| | | | | | pharmacopoeia |
| 120 | A | A | A | | |

The biochemical results are expressed according to the following classification:
Class A: IC50 less than 100 nM
Class B: IC50 between 100 nM and 1000 nM (or 1 μM)
Class C: IC50 between 1 μM and 5 μM
Class D: IC50 greater than 5 μM

### Cell Results

| | EC50 EOL- | EC50 MV4- | EC50 MOLM- | IC50 | IC50 |
|---|---|---|---|---|---|
| Example | 1 μM | 11 μM | 13 μM | phosphoBAD | phosphoBAD |
| No. | (lymphoma) | (myeloma) | (myeloma) | (JEKO) | (DND-41) |
| 7 | A | B | B | | |
| 9 | A | A | B | B | C |
| 10 | B | B | B | | |
| 11 | | | | B | |
| 12 | B | C | B | | |
| 19 | B | | B | | |
| 20 | B | | B | | |
| 21 | B | B | B | | |
| 22 | B | B | | C | |
| 23 | | | | B | B |
| 24 | B | B | | B | C |
| 25 | B | B | | C | B |
| 26 | | | | C | |
| 28 | B | B | | | C |
| 37 | | | | | C |
| 38 | A | B | B | | |
| 44 | A | A | | B | B |
| 52 | A | B | | | B |
| 56 | A | A | | | |
| 59 | B | B | | | C |
| 61 | B | B | | | C |
| 63 | B | B | | | B |
| 64 | B | B | | | B |
| 66 | A | B | | | B |
| 68 | A | B | | | A |
| 75 | B | B | | | C |
| 87 | B | B | | | |
| 89 | B | B | | | |
| 90 | B | B | | | |
| 106 | A | B | | | |
| 107 | B | B | | | |
| 120 | A | A | | | B |

The biochemical results are expressed according to the following classification:
Class A: EC50 or IC50 less than 100 nM
Class B: EC50 or IC50 between 100 nM and 1000 nM (or 1 μM)
Class C: EC50 or IC50 between 1 μM and 5 μM

1. A compound of general formula (I): 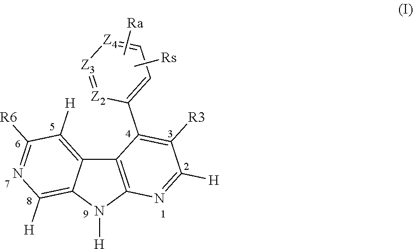
in which
Z₂, Z₃, Z₄, which may be identical or different, represent CH, CRa, CRs or N;
R3 is chosen from:
1. H;
2. halogen (F, Cl, Br, I);
3. CF₃, CHF;
4. OH
5. alkoxy in which the alkyl part is optionally mono-, di- or trisubstituted;
6. NH2, NH(alkyl), N(alkyl)₂ in which the alkyl part is optionally mono-, di- or trisubstituted;
7. C(O)Oalkyl optionally mono-, di- or trisubstituted;
8. CONH(alkyl), CON(alkyl)₂ in which the alkyl part is optionally mono-, di- or trisubstituted;
9. linear, branched or cyclic C₁-C₁₀ alkyl optionally comprising a heteroatom and optionally mono-, di- or trisubstituted;
10. aryl or heteroaryl optionally mono-, di- or trisubstituted;
R6 being a heteroaryl (5- or 6-membered with 1 to 4 heteroatoms chosen from N, S and O) linked to the azacarboline unit either via a C or via an N belonging to R6, R6 being optionally mono- or polysubstituted;
Ra necessarily being chosen from:
1. CONH₂,
2. CONHalkyl, CONHcycloalkyl optionally mono-, di- or trisubstituted;
3. CONHheterocycloalkyl optionally mono-, di- or trisubstituted;
4. CON(alkyl)₂ optionally mono-, di- or trisubstituted;
5. CON(alkyl)(heterocycloalkyl) optionally mono-, di- or trisubstituted;
6. CONHN(alkyl)₂ in which the alkyl part is optionally mono-, di- or trisubstituted;
7. C(O)heterocycloalkyl, the heterocycloalkyl radical containing at least one nitrogen atom linked to C(O); and being optionally mono-, di- or trisubstituted;
Rs being chosen from the following groups:
1. H;
2. F; Cl; Br; I
3. OH;
4. linear or branched O-alkyl(C₁-C₁₀) optionally mono- or polysubstituted;
5. NH₂;
6. N(alkyl(C₁-C₁₀) or cycloalkyl(C₃-C₇))₂, each group being optionally mono- or polysubstituted;
7. NHC(O)R3a;
8. N(alkyl(C1-C10)C(O)R3a;
9. NHS(O2)R3a;
10. N(alkyl(C1-C10)S(O2)R3a;
11. CO2R3a;
12. SR3a; S(O)R3a; S(O2)R3a
Ra and Rs possibly forming a 4- to 7-membered ring substituted with an oxo radical, comprising at least one nitrogen atom and optionally another heteroatom chosen from N, O and S and optionally substituted with one or more radicals chosen from oxo, F, Cl, Br, I, CF3, CHF2, alkyl, OH, Oalkyl, NO2, NH2, NHAlk and N(Alk)2 radicals;
R3a being chosen from:
1. F; Cl; Br; I
2. CF3;
3. linear, branched or cyclic C1-C10
4. C3-C7 cycloalkyl;
5. C2-C6 alkenyl;
6. C2-C6 alkynyl;
7. OH;
8. linear or branched (C1-C10) or cyclic (C3-C7) O-alkyl;
9. heterocycloalkyl (C3-C7);
10. NH2;
11. NH-(alkyl(C1-C10) or cycloalkyl(C3-C7));
12. N(alkyl(C1-C10) or cycloalkyl(C3-C7))2;
13. NH-(alkyl(C1-C10) or heterocycloalkyl (C3-C7));
14. N(alkyl(C1-C10) or heterocycloalkyl (C3-C7))2; and
racemic, enantiomeric or diastereoisomeric isomers, and pharmaceutically acceptable salts thereof.

2. The compound according to claim 1, wherein the possible substituents of R3, R6 and Ra are chosen from the groups R2a, R2b and R2c chosen, independently of each other, from:
1. F;
2. Cl;
3. Br;
4. I;
5. CF₃; CHF2
6. linear or branched C₁-C₁₀ alkyl optionally mono- or polysubstituted;
7. C₃-C₇ cycloalkyl optionally mono- or polysubstituted;
8. OH;
9. linear or branched O-alkyl(C₁-C₁₀) optionally mono- or polysubstituted;
10. O-cycloalkyl (C₃-C₇) optionally mono- or polysubstituted;
11. O-aryl optionally mono- or polysubstituted;
12. aryl optionally mono- or polysubstituted;
13. heteroaryl optionally mono- or polysubstituted;
14. heterocycloalkyl optionally mono- or polysubstituted;
15. NO₂;
16. NH₂;
17. NH-(alkyl(C₁-C₁₀) or cycloalkyl(C₃-C₇) or heterocycloalkyl), each group optionally mono- or polysubstituted;
18. N(alkyl(C₁-C₁₀) or cycloalkyl(C₃-C₇))₂, each group being optionally mono- or polysubstituted;
19. NHaryl or NH heteroaryl optionally mono- or polysubstituted
20. NHC(O)substituted;
21. N(alkyl(C₁-C₁₀)C(O)substituted;
22. NHS(O₂)substituted;
23. N(alkyl(C₁-C₁₀)S(O₂)substituted;
24. CO₂ substituted;
25. S substituted;
26. S(O₂)substituted;
27. S(O) substituted;
28. oxo (double bond O); and
racemic, enantiomeric or diastereoisomeric isomers, and pharmaceutically acceptable salts thereof.

3. The compound according to claim 2, wherein the optional substituents of all the substituted groups and of the groups Rs, R2a, R2b and R2c or the groups are chosen from:
1. F; Cl; Br; I
2. CF₃;
3. linear or branched C₁-C₁₀ alkyl
4. C₃-C₇ cycloalkyl;
5. C₂-C₆ alkenyl;
6. C₂-C₆ alkynyl;
7. OH;
8. linear or branched (C₁-C₁₀) or cyclic (C₃-C₇) O-alkyl;
9. heterocycloalkyl (C₃-C₇);
10. NH₂;
11. NH-(alkyl(C₁-C₁₀) or cycloalkyl(C₃-C₇));
12. N(alkyl(C₁-C₁₀) or cycloalkyl(C₃-C₇))₂;
13. NH-(alkyl(C₁-C₁₀) or heterocycloalkyl (C₃-C₇));
14. N(alkyl(C₁-C₁₀) or heterocycloalkyl (C₃-C₇))₂; and
racemic, enantiomeric or diastereoisomeric isomers, and pharmaceutically acceptable salts thereof.

4. The compound according to claim 1, wherein
Z₂, Z₃, Z₄, which may be identical or different, represent CH, CRa, CRs or N;
R3 is chosen from:
1—H
2—halogen (F, Cl, Br, I);
3—CF₃, CHF7;
4—OH
5—alkoxy in which the alkyl part is optionally mono-, di- or trisubstituted with R2a, R2b, R2c;
6—NH₂, NH(alkyl), N(alkyl)₂ in which the alkyl part is optionally mono-, di- or trisubstituted with R2a, R2b, R2c;
7—C(O)Oalkyl optionally mono-, di- or trisubstituted with R2a, R2b, R2c;
8—CONH(alkyl), CON(alkyl)₂ in which the alkyl part is optionally mono-, di- or trisubstituted with R2a, R2b, R2c;
9—linear, branched or cyclic C₁-C₁₀ alkyl optionally comprising a heteroatom and optionally mono-, di- or trisubstituted with R2a, R2b, R2c;
10—aryl or heteroaryl optionally mono-, di- or trisubstituted with R2a, R2b, R2c;
R6 being a heteroaryl (5- or 6-membered with 1 to 4 heteroatoms chosen from N, S and O) linked to the azacarboline unit either via a C or via an N belonging to R6, R6 being optionally mono- or polysubstituted with R2a, R2b, R2c;
Ra necessarily being:
1. CONH₂,
2. CONHalkyl, CONHcycloalkyl optionally mono-, di- or trisubstituted with R2a, R2b, R2c;
3. CONHheterocycloalkyl optionally mono-, di- or trisubstituted with R2a, R2b, R2c;
4. CON(alkyl)₂ optionally mono-, di- or trisubstituted with R2a, R2b, R2c;
5. CON(alkyl)(heterocycloalkyl) optionally mono-, di- or trisubstituted with R2a, R2b, R2c;
6. CONHN(alkyl)₂ in which the alkyl part is optionally mono-, di- or trisubstituted with R2a, R2b, R2c;
7. C(O)heterocycloalkyl, the heterocycloalkyl radical containing at least one nitrogen atom linked to C(O); and being optionally mono-, di- or trisubstituted;
Rs being chosen from the following groups:
1. H;
2. F; Cl; Br; I
3. OH;
4. linear or branched O-alkyl(C₁-C₁₀) optionally mono- or polysubstituted with identical or different groups R3a;
5. NH₂;
6. N(alkyl(C₁-C₁₀) or cycloalkyl(C₃-C₇))₂, each group being optionally mono- or polysubstituted with identical or different groups R3a;
7. NHC(O)R3a;
8. N(alkyl(C₁-C₁₀)C(O)R3a;
9. NHS(O₂)R3a;
10. N(alkyl(C₁-C₁₀)S(O₂)R3a;
11. CO₂R3a;
12. SR3a; S(O)R3a; S(O₂)R3a
Ra and Rs possibly forming a 5- to 6-membered ring substituted with an oxo radical, comprising at least one nitrogen atom and optionally substituted with one or more radicals chosen from oxo, F, Cl, Br, I, CF3, CHF2, alkyl, OH, Oalkyl, NO2, NH2, NHAlk and N(Alk)2 radicals;
the groups R2a, R2b or R2c are chosen, independently of each other, from:
1. F;
2. Cl;
3. Br;
4. I;
5. CF₃; CHF₂
6. linear or branched C₁-C₁₀ alkyl optionally mono- or polysubstituted with identical or different groups R3a;
7. C₃-C₇ cycloalkyl optionally mono- or polysubstituted with identical or different groups R3a;
8. OH;
9. linear or branched O-alkyl(C₁-C₁₀) optionally mono- or polysubstituted with identical or different groups R3a;
10. O-cycloalkyl (C₃-C₇) optionally mono- or polysubstituted with identical or different groups R3a;
11. O-aryl optionally mono- or polysubstituted with different groups R3a;
12. aryl optionally mono- or polysubstituted with identical or different groups R3a;
13. heteroaryl optionally mono- or polysubstituted with identical or different groups R3a;
14. heterocycloalkyl optionally mono- or polysubstituted with identical or different groups R3a;
15. NO₂
16. NH₂;
17. NH-(alkyl(C₁-C₁₀) or cycloalkyl(C₃-C₇) or heterocycloalkyl), each group optionally mono- or polysubstituted with identical or different groups R3a;
18. N(alkyl(C₁-C₁₀) or cycloalkyl(C₃-C₇))₂, each group being optionally mono- or polysubstituted with identical or different groups R3a;
19 NHaryl or NH heteroaryl optionally mono- or polysubstituted with identical or different groups R3a
20. NHC(O)R3a;
21. N(alkyl(C₁-C₁₀)C(O)R3a;
22. NHS(O₂)R3a;
23. N(alkyl(C₁-C₁₀)S(O₂)R3a;
24. CO₂R3a;
25. SR3a; S(O)R3a; S(O₂)R3a;
26. N(alkyl(Cₜ—C₁₀) or cycloalkyl(C₃-C₇))₂, each group being optionally mono- or polysubstituted with identical or different groups R3a;
27. oxo (double bond O);
the possible substituents of the groups R2a, R2b and R2c in which the groups R3a are chosen from:
1. F; Cl; Br; I
2. CF₃;
3. linear or branched C₁-C₁₀ alkyl
4. C₃-C₇ cycloalkyl;
5. C₂-C₆ alkenyl;
6. C₂-C₆ alkynyl;
7. OH;
8. linear or branched (C₁-C₁₀) or cyclic (C₃-C₇) O-alkyl;
9. heterocycloalkyl (C₃-C₇);
10. NH₂;
11. NH-(alkyl(C₁-C₁₀) or cycloalkyl(C₃-C₇));
12. N(alkyl(C₁-C₁₀) or cycloalkyl(C₃-C₇))₂;
13. NH-(alkyl(C₁-C₁₀) or heterocycloalkyl (C₃-C₇));
14. N(alkyl(C₁-C₁₀) or heterocycloalkyl (C₃-C₇))₂; and
racemic, enantiomeric or diastereoisomeric isomers, and pharmaceutically acceptable salts thereof.

5. The compound according to claim 1, having the formula Ia: 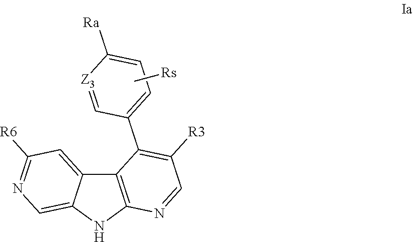
in which Z2 represents CH, Z3 represents CH or N and Z4 represents —C-Ra.

6. The compound according to claim 1, having the formula Ib: 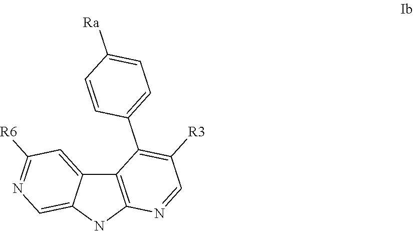
in which Rs represents a hydrogen atom, Z2 and Z3 represent CH and Z4 represents —C-Ra.

7. The compound according to claim 1, having the formula Ic: 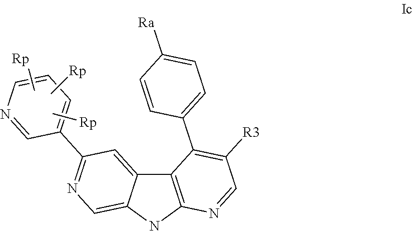
in which Rs represents a hydrogen atom, Z2 and Z3 represent CH, Z4 represents —C-Ra and R6 represents a pyridyl radical optionally mono- or polysubstituted with one or more identical or different radicals Rp chosen from F, Cl, Br, I, CF3, CHF2, alkyl, OH, Oalkyl, NO2, NH2, NHAlk and N(Alk)2 radicals.

8. The compound according to claim 1, having the formula Id: 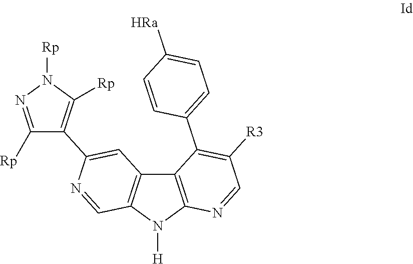
in which Rs represents a hydrogen atom, Z2 and Z3 represent CH, Z4 represents —C-Ra and R6 represents a pyrazolyl radical optionally mono- or polysubstituted with one or more identical or different radicals Rp chosen from F, Cl, Br, I, CF3, CHF2, alkyl, OH, Oalkyl, NO2, NH2, NHAlk and N(Alk)2 radicals.

9. The compound according to claim 1, wherein said compound is chosen from the following:
4-[3-fluoro-6-(pyridin-3-yl)-9H-pyrrolo[2,3-b:5,4-c′]dipyridin-4-yl]-N-(1H-tetrazol-5-yl-methyl)benzamide
[(3R)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
{4-[3-fluoro-6-(pyridin-3-yl)-9H-pyrrolo[2,3-b:5,4-c′]dipyridin-4-yl]phenyl}[(3aS,6aS)-5-methylhexahydropyrrolo[3,4-b]pyrrol-1(2H)-yl]methanone
N-[2-(acetylamino)ethyl]-4-[3-fluoro-6-(pyridin-3-yl)-9H-pyrrolo[2,3-b:5,4-c′]dipyridin-4-yl]-benzamide
4-[3-fluoro-6-(pyridin-3-yl)-9H-pyrrolo[2,3-b:5,4-c′]dipyridin-4-yl]-N-[3-(2-oxopyrrolidin-1-yl)propyl]benzamide
4-[3-fluoro-6-(pyridin-3-yl)-9H-pyrrolo[2,3-b:5,4-c′]dipyridin-4-yl]-N-[2-(phenylamino)ethyl]-benzamide
N-[(1-ethylpyrrolidin-2-yl)methyl]-4-[3-fluoro-6-(pyridin-3-yl)-9H-pyrrolo[2,3-b:5,4-c′]dipyridin-4-yl]benzamide
N-[3-(dimethylamino)-2,2-dimethylpropyl]-4-[3-fluoro-6-(pyridin-3-yl)-9H-pyrrolo[2,3-b:5,4-c′]dipyridin-4-yl]benzamide
N-{[(2S)-1-ethylpyrrolidin-2-yl]methyl}-4-[3-fluoro-6-(pyridin-3-yl)-9H-pyrrolo[2,3-b:5,4-c′]dipyridin-4-yl]benzamide
N-(1-ethylpiperidin-3-yl)-4-[3-fluoro-6-(pyridin-3-yl)-9H-pyrrolo[2,3-b:5,4-c′]dipyridin-4-yl]-benzamide
4-[3-fluoro-6-(pyridin-3-yl)-9H-pyrrolo[2,3-b:5,4-c′]dipyridin-4-yl]-N-[2-(2-methylpiperidin-1-yl)ethyl]benzamide
4-[3-fluoro-6-(pyridin-3-yl)-9H-pyrrolo[2,3-b:5,4-c′]dipyridin-4-yl]-N-(1-methylazetidin-3-yl)benzamide
[3-(dimethylamino)piperidin-1-yl]{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-[2-methyl-2-(pyrrolidin-1-yl)propyl]benzamide
N-[3-(dimethylamino)propyl]-4-[3-fluoro-6-(pyridin-3-yl)-9H-pyrrolo[2,3-b:5,4-c′]dipyridin-4-yl]-N-methylbenzamide
N-[2-(azepan-1-yl)ethyl]-4-[3-fluoro-6-(pyridin-3-yl)-9H-pyrrolo[2,3-b:5,4-c′]dipyridin-4-yl]-benzamide
4-[3-fluoro-6-(pyridin-3-yl)-9H-pyrrolo[2,3-b:5,4-c′]dipyridin-4-yl]-N-[2-(1-methylpiperidin-4-yl)ethyl]benzamide
4-[3-fluoro-6-(pyridin-3-yl)-9H-pyrrolo[2,3-b:5,4-c′]dipyridin-4-yl]-N-{2-[(methyl-sulfonyl)amino]ethyl}benzamide
4-[3-fluoro-6-(pyridin-3-yl)-9H-pyrrolo[2,3-b:5,4-c′]dipyridin-4-yl]-N-[2-(pyrrolidin-1-yl)propyl]benzamide
4-[3-fluoro-6-(pyridin-3-yl)-9H-pyrrolo[2,3-b:5,4-c′]dipyridin-4-yl]-N-methyl-N-[(1-methylpiperidin-2-yl)methyl]benzamide
4-[3-fluoro-6-(pyridin-3-yl)-9H-pyrrolo[2,3-b:5,4-c′]dipyridin-4-yl]-N-[2-(1-methylpyrrolidin-2-yl)ethyl]benzamide
N-[2-(dipropan-2-ylamino)ethyl]-4-[3-fluoro-6-(pyridin-3-yl)-9H-pyrrolo[2,3-b:5,4-c′]dipyridin-4-yl]benzamide
N-[2-(dimethylamino)ethyl]-N-ethyl-4-[3-fluoro-6-(pyridin-3-yl)-9H-pyrrolo[2,3-b:5,4-c′]dipyridin-4-yl]benzamide
N-[1-(dimethylamino)propan-2-yl]-4-[3-fluoro-6-(pyridin-3-yl)-9H-pyrrolo[2,3-b:5,4-c′]dipyridin-4-yl]benzamide
[(3S)-3-(dimethylamino)pyrrolidin-1-yl]{4-[3-fluoro-6-(pyridin-3-yl)-9H-pyrrolo[2,3-b:5,4-c′]dipyridin-4-yl]phenyl}methanone
4-[3-fluoro-6-(pyridin-3-yl)-9H-pyrrolo[2,3-b:5,4-c′]dipyridin-4-yl]-N-methyl-N-(1-methylpyrrolidin-3-yl)benzamide
N-[2-(diethylamino)ethyl]-4-[3-fluoro-6-(pyridin-3-yl)-9H-pyrrolo[2,3-b:5,4-e]dipyridin-4-yl]-N-methylbenzamide
{4-[3-fluoro-6-(pyridin-3-yl)-9H-pyrrolo[2,3-b:5,4-c′]dipyridin-4-yl]phenyl}[4-(2-methoxyethyl)piperazin-1-yl]methanone
4-[3-fluoro-6-(pyridin-3-yl)-9H-pyrrolo[2,3-b:5,4-c′]dipyridin-4-yl]-N-[(3-methyl-1H-pyrazol-4-yl)methyl]benzamide
{4-[3-fluoro-6-(pyridin-3-yl)-9H-pyrrolo[2,3-b:5,4-e]dipyridin-4-yl]phenyl}(2-methyl-octahydro-5H-pyrrolo[3,4-c]pyridin-5-yl)methanone
N-[4-(dimethylamino)butyl]-4-[3-fluoro-6-(pyridin-3-yl)-9H-pyrrolo[2,3-b:5,4-c′]dipyridin-4-yl]benzamide
4-[3-fluoro-6-(pyridin-3-yl)-9H-pyrrolo[2,3-b:5,4-c′]dipyridin-4-yl]-N-(1H-imidazol-2-yl-methyl)benzamide
{4-[3-fluoro-6-(pyridin-3-yl)-9H-pyrrolo dipyridin-4-yl]phenyl}(7-methyl-2,7-diazaspiro[4.4]non-2-yl)methanone
4-[3-fluoro-6-(pyridin-3-yl)-9H-pyrrolo[2,3-b:5,4-c′]dipyridin-4-yl]-N-[2-(pyridin-2-ylamino)-ethyl]benzamide
N-ethyl-4-[3-fluoro-6-(pyridin-3-yl)-9H-pyrrolo[2,3-b:5,4-c′]dipyridin-4-yl]-N-[(1-methylpyrrolidin-3-yl)methyl]benzamide
1,3′-bipyrrolidin-1′-yl{4-[3-fluoro-6-(pyridin-3-yl)-9H-pyrrolo[2,3-b:5,4-c′]dipyridin-4-yl]-phenyl}methanone
4-[3-fluoro-6-(pyridin-3-yl)-9H-pyrrolo[2,3-b:5,4-c′]dipyridin-4-yl]-N-methyl-N-(1-methylpiperidin-4-yl)benzamide
4-[3-fluoro-6-(pyridin-3-yl)-9H-pyrrolo[2,3-b:5,4-c′]dipyridin-4-yl]-N-[(2-hydroxypyridin-4-yl)methyl]benzamide
N-[2-(ethylamino)ethyl]-4-[3-fluoro-6-(pyridin-3-yl)-9H-pyrrolo[2,3-b:5,4-c′]dipyridin-4-yl]-benzamide
4-[3-fluoro-6-(pyridin-3-yl)-9H-pyrrolo[2,3-b:5,4-c′]dipyridin-4-yl]-N-[2-(methylamino)ethyl]-benzamide
N-[(1-aminocyclopropyl)methyl]-4-[3-fluoro-6-(pyridin-3-yl)-9H-pyrrolo[2,3-b:5,4-c′]dipyridin-4-yl]benzamide
N-(3-amino-2,2-difluoropropyl)-4-[3-fluoro-6-(pyridin-3-yl)-9H-pyrrolo[2,3-b:5,4-c′]dipyridin-4-yl]benzamide
N-(2-amino-3,3,3-trifluoro-2-methylpropyl)-4-[3-fluoro-6-(pyridin-3-yl)-9H-pyrrolo[2,3-b:5,4-c′]dipyridin-4-yl]benzamide
N-[(1R,2R)-2-aminocyclohexyl]-4-[3-fluoro-6-(pyridin-3-yl)-9H-pyrrolo[2,3-b:5,4-c′]dipyridin-4-yl]benzamide
N-[(1S,2S)-2-aminocyclohexyl]-4-[3-fluoro-6-(pyridin-3-yl)-9H-pyrrolo[2,3-b:5,4-c′]dipyridin-4-yl]benzamide
N-[(1S,2S)-2-aminocyclopentyl]-4-[3-fluoro-6-(pyridin-3-yl)-9H-pyrrolo[2,3-b:5,4-c′]dipyridin-4-yl]benzamide
N-[(1R,2R)-2-aminocyclopentyl]-4-[3-fluoro-6-(pyridin-3-yl)-9H-pyrrolo[2,3-b:5,4-c′]dipyridin-4-yl]benzamide
4-[3-fluoro-6-(1-methyl-1H-pyrazol-4-yl)-9H-pyrrolo[2,3-b:5,4-e′]dipyridin-4-yl]-N-(4-methylpiperazin-1-yl)benzamide
4-[3-fluoro-6-(1-methyl-1H-pyrazol-4-yl)-9H-pyrrolo[2,3-b:5,4-c′]dipyridin-4-yl]-N-(1-methylpiperidin-4-yl)benzamide
4-[3-fluoro-6-(1-methyl-1H-pyrazol-4-yl)-9H-pyrrolo[2,3-b:5,4-c′]dipyridin-4-yl]-N-{2-[(3R)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-{2-[(3S)-3-hydroxypyrrolidin-1-yl]ethyl}benzamide
4-[3-fluoro-6-(1-methyl-1H-pyrazol-4-yl)-9H-pyrrolo[2,3-b:5,4-c′]dipyridin-4-yl]-N-(2-hydroxyethyl)benzamide
N-[(1S,2S)-2-aminocyclohexyl]-4-[3-fluoro-6-(1-methyl-1H-pyrazol-4-yl)-9H-pyrrolo[2,3-b:5,4-c′]dipyridin-4-yl]benzamide
N-[(1S,2S)-2-(diethylamino)cyclohexyl]-4-[3-fluoro-6-(1-methyl-1H-pyrazol-4-yl)-9H-pyrrolo-[2,3-b:5,4-c′]dipyridin-4-yl]benzamide
N-[(1S,2S)-2-(ethylamino)cyclohexyl]-4-[3-fluoro-6-(1-methyl-1H-pyrazol-4-yl)-9H-pyrrolo-[2,3-b:5,4-c′]dipyridin-4-yl]benzamide
4-[3-fluoro-6-(1-methyl-1H-pyrazol-4-yl)-9H-pyrrolo[2,3-b:5,4-c′]dipyridin-4-yl]benzamide
4-[6-(5-chloro-1-methyl-1H-pyrazol-4-yl)-3-fluoro-9H-pyrrolo[2,3-b:5,4-c′]dipyridin-4-yl]-N-(4-methylpiperazin-1-yl)benzamide
4-[6-(5-chloro-1-methyl-1H-pyrazol-4-yl)-3-fluoro-9H-pyrrolo[2,3-b:5,4-c′]dipyridin-4-yl]-N-(1-methylpiperidin-4-yl)benzamide
N-[2-(dimethylamino)ethyl]-4-[3-(2-methoxyethoxy)-6-(pyridin-3-yl)-9H-pyrrolo[2,3-b:5,4-c′]dipyridin-4-yl]benzamide
N-[2-(dimethylamino)ethyl]-5-[3-fluoro-6-(pyridin-3-yl)-9H-pyrrolo[2,3-b:5,4-c′]dipyridin-4-yl]pyridine-2-carboxamide
N-[2-(dimethylamino)ethyl]-2-fluoro-4-[3-fluoro-6-(pyridin-3-yl)-9H-pyrrolo[2,3-b:5,4-c]dipyridin-4-yl]benzamide

10. A process for preparing the compound according to claim 1, comprising the general scheme below: 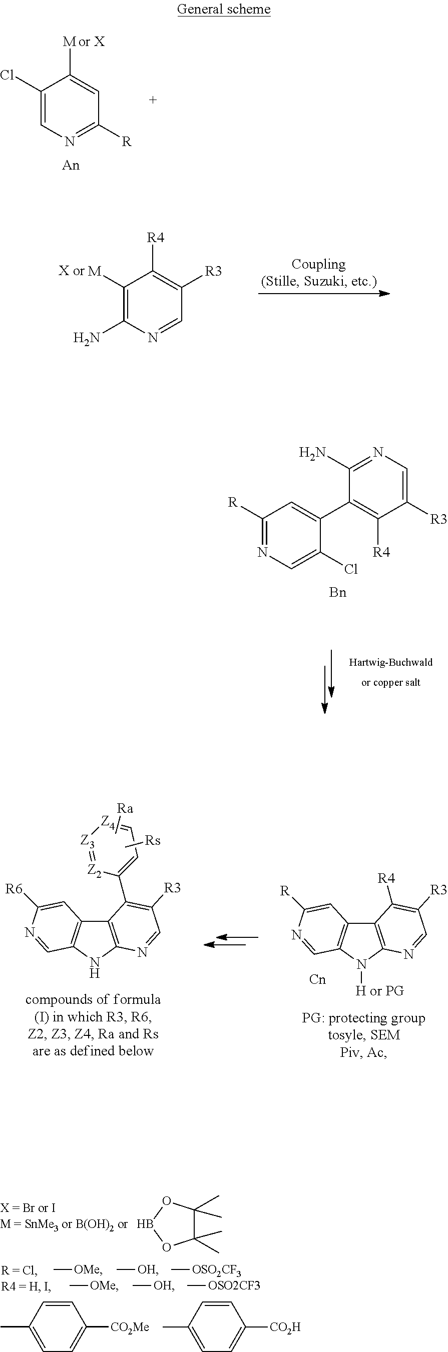
in which the substituents R3, R6; Z2, Z3, Z4 Ra and Rs have the meanings indicated in claim 1.

11. A pharmaceutical composition comprising the compound of claim 1, and pharmaceutically acceptable salts thereof or a prodrug thereof.

12. A pharmaceutical composition containing as active principle a compound according to claim 1, and at least one pharmaceutically compatible excipient.

13. A method of treating cancer in a patient in need thereof comprising administering to said patient a therapeutically effective amount of the pharmaceutical composition of claim 11.

14. A product having one of the following formulas: 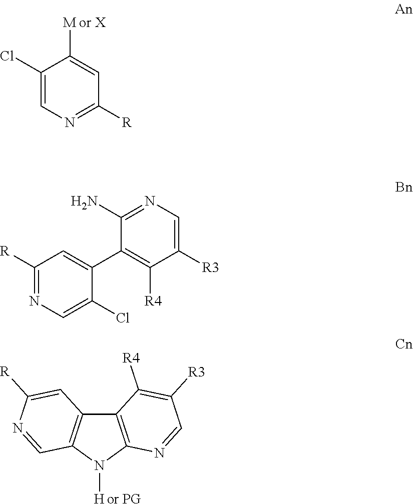
in which X, M, R, R3, R4 and PG have the definitions indicated below: [chemical structure]
